(19) United States
(12) Patent Application Publication
(10) Pub. No.: US 2023/0096897 A1
(43) Pub. Date: Mar. 30, 2023

(54) Lymphocyte Population and Methods for Producing Same

(71) Applicant: AVM Biotechnology, LLC, Seattle, WA (US)

(72) Inventors: Theresa Deisher, Seattle, WA (US); Scot Wayne McKay, Seattle, WA (US)

(73) Assignee: AVM Biotechnology, LLC, Seattle, WA (US)

(21) Appl. No.: 17/801,293

(22) PCT Filed: Feb. 26, 2021
(86) PCT No.: PCT/US2021/019773

§ 371 Date: Aug. 22, 2022

Related U.S. Application Data
(60) Provisional application No. 63/123,054, filed on Dec. 9, 2020.
(60) Provisional application No. 63/009,050, filed on Apr. 13, 2020.
(60) Provisional application No. 62/983,005, filed on Feb. 28, 2020.

(51) Int. Cl. A61K 31/573; C12N 5/0783; C12N 15/62; C12N 15/85; A61K 45/06; A61P 35/00

(52) U.S. Cl. CPC A61K 31/573; C12N 5/0646; C12N 15/625; C12N 15/85; A61K 45/06; A61P 35/00; C12N 2501/39; C12N 2800/107

(57) ABSTRACT

This invention pertains to a novel population of lymphocytes, methods for producing these, and their use in the treatment of diseases.

### FIELD

This disclosure pertains to novel populations of lymphocytes and immune cells, methods for producing these, and their use in the treatment of diseases. More particularly, the disclosure relates to methods for producing novel populations of natural killer T cells (NKT cells), T cells, and dendritic cells using high dose glucocorticoids and glucocorticoid receptor agonists.

### BACKGROUND

The present authors have previously found that high concentrations of glucocorticoids could be used to condition patients to enhance the efficacy of cellular immunotherapies such as adoptive T cell therapy; described in International patent application PCT/US2018/025517 (published as WO2018/183927). In that application, the authors noted the toxicities associated with chemotherapy and radiation mediated preconditioning, which is believed to non-selectively destroy the cellularity of the spleen. The authors provided glucocorticoids (a subclass of steroids) and other non-toxic lymphodepleting agents, at acute doses, to benefit cancer patients who receive cellular immunotherapies.

In international patent application PCT/US2019/054395 the present authors have also described the use of high concentrations of glucocorticoids to cause lymphodepletion of peripheral blood lymphocytes without substantially affecting the cell count of other cells. In that application, the authors reported that high concentrations of glucocorticoids can deplete peripheral blood lymphocytes including, for example, islet-specific autoreactive T-cells responsible for diabetes autoimmunity, but spares neutrophils, platelets, RBCs and stem cells (both HSCs and MSCs). The authors provided glucocorticoids as a non-myeloablative regimen that can perform a safe immunologic reset with efficacy comparable to chemotherapy.

Reducing cytotoxic chemotherapy use is a top priority goal of the National Cancer Institute. Carcinomas, often called solid tumors, represent 80-90% of total cancers, but have proven difficult to target with newer cancer therapy developments. Chimeric antigen receptor (CAR) T-cell therapy has shown remarkable success in the treatment of CD expressing B-cell acute lymphocytic leukemia. However, there are a number of obstacles that limit CAR T-cell therapy for solid tumors: ineffective trafficking to the tumor as well immunosuppressive microenvironments in solid tumors limit T-cell efficacy. In addition, CAR T therapies have been associated with serious adverse effects, including cytokine release syndrome (CRS), neuroedema, and graft versus host disease (GvHD).

Natural Killer T Cells (NKTs) are a heterogeneous group of T cells that share properties of both T cells and natural killer (NK) cells. In contrast to conventional T cells, NKTs are functionally mature when they exit the thymus, primed for rapid cytokine production. NKTs can directly kill CD1d expressing cancer cells and tumor microenvironment macrophages, rapidly produce and release immune activating cytokines such as IFNgamma and IL-4, and activate other immune cells such as dendritic cells (DCs), NK cells, and B and T lymphocytes. Clinically, invariant NKTs (iNKTs) have been used against a variety of different cancers, either by injection of ‘autologous culture activated iNKTs’, by administering alpha Gal Cer (an NKT activator) loaded dendritic cells or monocytes to activate endogenous NKTs, or by administering NKT activator antibodies or ligands such as KRN7000, a synthetic analogue of alpha Gal Cer.

However, none of these methods used to induce iNKT production have been demonstrated to be effective in cancer patients; iNKT levels are reduced in cancer patients and clinical trials have been disappointing. iNKT levels are similarly low in the elderly (Tarazona et al, 2003, which is hereby incorporated by reference in its entirety). Use of ‘autologous culture activated NKTs’ in melanoma was effective in 3 of 9 patients, with outcome directly associated with the number of tumor infiltrating NKTs (Wolf et al, 2018 and Nair et al, 2017, which is hereby incorporated by reference in its entirety). This approach, however, was also limited by the low numbers of NKTs in cancer patients, and by the plasticity of iNKT to move between IFN gamma type 1 and tumor promoting IL-4 type 2.

In cancer treatment, kinase inhibitors (KIs) are well tolerated compared to conventional cytotoxic chemotherapy. However, significant toxicities are still associated with the kinase inhibitors including fatigue, hypertension, rash, impaired wound healing, myelosuppression, and diarrhea, and abnormalities in thyroid function, bone metabolism, linear growth, gonadal function, fetal development, adrenal function, and glucose metabolism. Many patients require dose-reduction because of the toxicities of the KIs, which must be taken chronically (Lodisch et al, 2013, which is hereby incorporated by reference in its entirety). Additionally, resistance to the KIs is common and time-dependent with treatment (Bhullar 2018, which is hereby incorporated by reference in its entirety).

Despite efforts to reduce the toxicities associated with cancer treatments, the physical toll and medical costs to manage these toxicities remain a significant concern. For example, up to 41% of blood cancer patients choose to stop taking the new kinase/proteasome inhibitors or biologics due to the physical and financial toxicities associated with these drugs (Mato 2018, Kadri 2017, Mato 2016 and Barrett 2010, each of which is hereby incorporated by reference in its entirety).

T cells are a type of lymphocyte that play a key role in the immune response. T cells are distinguished from other types of lymphocytes by the presence of T-cell receptors on their cell surface. T-cell receptors (TCRs) are responsible for recognizing fragments of antigen bound to major histocompatibility complex (MHC) molecules, and are heterodimers of two different protein chains. In humans, in 95% of T cells the TCR consists of an alpha (a) chain and a beta (β) chain (encoded by TRA and TRB, respectively), whereas in 5% of T cells the TCR consists of gamma and delta (γ/δ) chains (encoded by TRG and TRD, respectively). This ratio changes in diseased states (such as leukemia).

In contrast to MHC-restricted alpha beta T cells, gamma delta T cells do not require antigen processing and major-histocompatibility-complex (MHC) presentation of peptide epitopes for activation, although some recognize MHC class Ib molecules. Some gamma delta T cells recognise markers of cellular stress resulting from infection or tumorigenesis. Gamma delta T cells are also believed to have a role in recognition of lipid antigens.

Gamma delta T cells display broad functional plasticity following recognition of infected/transformed cells by production of cytokines (IFN-γ, TNF-α, IL-17) and chemokines (RANTES, IP-10, lymphotactin), cytolysis of infected or transformed target cells (perforin, granzymes, TRAIL), and interaction with other cells. Gamma delta T cells have been shown to be capable of recognising and lysing diverse cancers in an MHC-unrestricted manner, to have a protective function in infectious disease, and to be associated with progression and prognosis in various infectious diseases (Gogoi et al, 2013; Pauza et al, 2018; Zheng et al, 2012; Dong et al, 2018; Zhao et al 2018; all hereby incorporated by reference in their entirety). Some gamma delta T cells can also behave as antigen presenting cells in some circumstances (Himoudi et al, 2012). Gamma delta T cells are thus of considerable interest in immunotherapy development.

Dendritic cells are bone marrow-derived leukocytes, and are the most potent antigen-presenting cells of the mammalian immune system. Dendritic cells are frequently classified into conventional dendritic cell (cDC) and plasmacytoid dendritic cell (pDC) subsets. Dendritic cells exist primarily in two basic functional states: “immature” and “mature”. Activation (maturation) of dendritic cells turns on metabolic, cellular, and gene transcription programs allowing DC to migrate from peripheral tissues to T-dependent areas in secondary lymphoid organs, where T lymphocyte-activating antigen presentation may occur (Patente et al, 2018; hereby incorporated by reference in its entirety).

The main function of dendritic cells is to process antigen material and present it on the cell surface to T cells thus initiating adaptive immune responses. Dendritic cells also produce polarizing cytokines that promote pathogen-specific effector T cell differentiation and activation, and can promote self-tolerance by secreting tolerogenic cytokines that induce the differentiation of regulatory T cells. In view of these immune regulatory functions, dendritic cells are of considerable interest in immunotherapy development, for treatment of conditions including cancer, autoimmune diseases, and infection. For example, CD11b positive dendritic cells have been associated with reduced severity of, or protection, from Influenza A (H1N1) infection, and Respiratory Syncytial Virus (Lee et al, 2018; Malloy et al, 2017; both hereby incorporated by reference in their entirety).

Coronavirus disease 2019 (COVID-19) is an infectious disease caused by severe acute respiratory syndrome coronavirus 2 (SARS-CoV-2). While the majority of cases result in mild symptoms (which may include fever, cough, and shortness of breath), some progress to viral pneumonia and multi-organ failure. The COVID-19 outbreak was declared a pandemic by the World Health Organisation (WHO) in March 2020. As of April 2020, the number of confirmed global cases exceeded 1 million, with resulting deaths of over 50,000. As of April 2020, no vaccine or specific antiviral treatments existed for COVID-19, with management of the disease focused on treatment of symptoms and supportive care.

A need exists for further treatments for cancer, autoimmune disorders, and infectious (also called microbial) diseases that are safer and associated with fewer toxicities and/or greater efficacy than currently available therapies. Treatments that are simpler, less toxic, and less costly are desired.

### SUMMARY

The present invention is based on the surprising finding that while high doses of glucocorticoids act to cause lymphodepletion of many types of peripheral blood lymphocytes, they also induce production/activation/mobilisation of a novel population of Natural Killer T (NKT) cells. In addition to presenting with the properties of known NKT cells, this novel population of NKT cells is able to directly engulf cancer cells, thus expanding the potential of high concentrations of glucocorticoids as a therapeutic treatment for solid cancers.

The present authors have also discovered that, following high dose administration, glucocorticoid molecules can bind and block intercellular adhesion molecules such as ICAM3. The binding is cooperative and up to 26 molecules bind the first Ig domain of ICAM3. ICAM3 is expressed at substantial levels on cells such as lymphocytes, monocytes and neutrophils, as well as on cancer cell types such as melanoma and osteosarcoma.

Accordingly, in a first aspect, the invention provides a method of producing a population of natural killer T cells (NKT cells), the method comprising administering to a subject a glucocorticoid-receptor (GR) modulating agent or ICAM3 modulating agent (which may be a glucocorticoid, such as dexamethasone) at a dose equivalent to about at least 6 mg/kg human equivalent dose (HED) of dexamethasone base, wherein the glucocorticoid induces the population of NKT cells in the subject. The NKT cells of the invention exhibit a novel pattern of marker expression. In some embodiments, the population of NKT cells are characterized in that at least 60, 70, 80, 90, 95, 96, 97, 98, or 99% of the cells express CD3, CD4, CD8, CD45, CD49b, CD56, CD62L, NK1.1, Ly6G, Sca1, and/or TCR gamma/delta; and/or do not express: C-kit, B220, FoxP3, and/or TCR alpha/beta. In some embodiments, the NKT cells express CD3, CD4, CD45, CD49b, CD56, CD62L, NK1.1, Ly6G, and Sca1. In some embodiments, the NKT cells express CD3, CD4, CD45, CD56, CD62L, NK1.1, Ly6G, and Sca1. In some embodiments, the NKT cells express CD3, CD4, CD45, CD49b, CD62L, NK1.1, Ly6G, and Sca1. In some embodiments, the NKT cells express CD3, CD4, CD45, CD56, CD62L, NK1.1, and Ly6G. In some embodiments, the NKT cells express CD3, CD4, CD45, CD49b, CD62L, NK1.1, and Ly6G. In some embodiments, the population of NKT cells are characterized in that at least 60, 70, 80, 90, 95, 96, 97, 98, or 99% of the cells express CD3, CD4, CD8, CD45, CD49b, CD56, CD62L, NK1.1, Ly6G, and/or TCR gamma/delta; and/or do not express: C-kit, B220, FoxP3, and/or TCR alpha/beta. In some embodiments, the population of NKT cells are characterized in that at least 60, 70, 80, 90, 95, 96, 97, 98, or 99% of the cells express CD3, CD4, CD8, CD45, CD56, CD62L, NK1.1, Ly6G, and/or TCR gamma/delta; and/or do not express: C-kit, B220, FoxP3, and/or TCR alpha/beta. In some embodiments, the NKT cells do not express C-kit, B220, FoxP3, or TCR alpha/beta. In some embodiments, the NKT cells do not express Sca1. The NKT cells may express CD8. The NKT cells may not express CD8. The NKT cells may express CD4. The NKT cells may not express CD4. The NKT cells may express CD4 and CD8; and/or express Ly6G.

In some preferred embodiments the NKT cells of the disclosure may express CD3, CD45, and/or CD56. In some such embodiments, the NKT cells of the disclosure may be CD3+/bright or CD3+/very bright, and/or CD45+/dim, and/or CD56+.

The NKT cells may be described as
CD4+/very bright;
CD8+/dim;
CD3+/very bright;
CD45+/dim;
Sca1+/very bright;
CD44+/−;
CD69+/−;
CD25+/−;
TCR gamma delta+; and/or
CDd49b+ or CD56+/bright.
   The NKT cells may be described as having these properties in naïve subjects. The NKT cells may be described as having these properties in a tumour/cancerous or autoimmune state.

The expression levels of the cell markers can be determined relative to the average expression level in a population of reference NKT cells, derived from a common source, which have not been contacted with the glucocorticoid-receptor (GR) modulating agent or ICAM3 modulating agent. Expression of the markers can be measured by flow cytometry, e.g. performed using the equipment, reagents, and/or conditions described herein (taken in isolation or in combination). The glucocorticoid-receptor (GR) modulating agent or ICAM3 modulating agent may be a glucocorticoid. In some embodiments, the glucocorticoid is selected from the group consisting of: dexamethasone, hydrocortisone, methylprednisolone, prednisone, prednisolone, prednylidene, cortisone, budesonide, betamethasone, flumethasone and beclomethasone.

In preferred embodiments, the glucocorticoid is selected from the group consisting of: dexamethasone, betamethasone, and methylprednisone (preferably dexamethasone or betamethasone).

In some embodiments, the glucocorticoid is selected from the group consisting of dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone isothiocyanatoethyl thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone.

In some embodiments, the glucocorticoid is dexamethasone, which is dexamethasone sodium phosphate.

The methods of the invention can involve the administration of a particular glucocorticoid dose. In some embodiments, the glucocorticoid is administered at a dose equivalent to about:
6-12 mg/kg human equivalent dose (HED) of dexamethasone base;
at least 6 mg/kg human equivalent dose (HED) of dexamethasone base;
at least 12 mg/kg human equivalent dose (HED) of dexamethasone base;
at least 15 mg/kg human equivalent dose (HED) of dexamethasone base;
at least 18 mg/kg human equivalent dose (HED) of dexamethasone base;
at least 21 mg/kg human equivalent dose (HED) of dexamethasone base;
at least 24 mg/kg human equivalent dose (HED) of dexamethasone base;
up to 45 mg/kg human equivalent dose (HED) of dexamethasone base.

In some preferred embodiments, the glucocorticoid is administered at a dose equivalent to about at least 18 mg/kg human equivalent dose (HED) of dexamethasone base. In some other preferred embodiments, the glucocorticoid is administered at a dose equivalent to about at least 15-18 mg/kg human equivalent dose (HED) of dexamethasone base.

The glucocorticoid dose can be defined as a human equivalent dose (HED) of dexamethasone having a mg/kg value from a range of mg/kg values, wherein said range is bound by two of the mg/kg values set forth in parts i) to viii) above. For instance, the glucocorticoid dose can be defined as a dexamethasone HED of 6-45 mg/kg. In another example, the glucocorticoid dose can be defined as a dexamethasone HED of 12-24 mg/kg.

The glucocorticoid may be administered as a single acute dose, or as a total dose given over about a 72 hour period. Moreover, the method may comprise administering one or more further doses of a glucocorticoid. In some embodiments, one or more further doses are administered: between 24 hours and 120 hours after a preceding glucocorticoid administration; between 24 hours and 48 hours after a preceding glucocorticoid administration; between 72 hours and 120 hours after a preceding glucocorticoid administration; every 24, 48, 72, 96, 120, 144, or 168 hours after a first glucocorticoid administration; once every week after a first glucocorticoid administration; once every two weeks after a first glucocorticoid administration; once monthly after a first glucocorticoid administration; or twice weekly after a first glucocorticoid administration.

The invention may include steps for NKT cell activation. Thus, in some embodiments, the methods may further comprise a step of administering an NKT cell activator to the subject. The NKT cell activator may be selected from the group consisting of: alpha GalCer, Sulfatide, or an NKT-activating antibody. The NKT cell activator may be alpha GalCer loaded dendritic cells or monocytes. The NKT cell activator may be administered within or around 1-48 hours after administration of glucocorticoid. The NKT cell activator may be administered within or around 48 hours after administration of glucocorticoid.

In some embodiments, the subject is mammalian, e.g. a human being.

The subject may have, or be suspected of having (or have been diagnosed with) cancer, an autoimmune disease, or infectious disease (also called microbial disease). The cancer may be a solid tumour. Alternatively, the cancer may be a lymphoma, preferably a B cell lymphoma or a T cell lymphoma. In some preferred embodiments, the cancer may be non-Hodgkin lymphoma.

The cancer may be selected from the group consisting of: squamous cell cancer (such as epithelial squamous cell cancer); lung cancer, including small-cell lung cancer, non-small cell lung cancer, adenocarcinoma of the lung and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; and head and neck cancer.

The NKT cells of the invention may treat cancer via tumour infiltration. The NKT cells of the invention may treat cancer via release of immune activating cytokines. The NKT cells of the invention may treat cancer may engulf and kill cancer cells. The NKT cells of the invention may treat cancer by promoting infiltration of other immune cells into the tumour. The NKT cells of the invention may treat cancer via CD1d-directed apoptosis. The NKT cells of the invention may treat cancer via tumour necrosis. The NKT cells of the invention may treat cancer by recognizing high levels of phosphoantigens made by tumor cells via expression of the gamma-delta T cell receptor on the NKT cells of the present invention. Thus, in some embodiments, the invention provides methods of causing tumour necrosis by inducing or administering the NKT cells of the invention. In some embodiments, the invention provides methods of causing CD1d-directed apoptosis of cancer cells by inducing or administering the NKT cells of the invention. In some embodiments, the invention provides methods of engulfing and/or killing cancer cells using the NKT cells of the invention. In some embodiments, the invention provides methods of activation of the gamma-delta expressing NKT cells by cancer cell phosphoantigens which then recognize and kill cancer cells via the NK receptor(s) on the NKT cells.

In embodiments where the subject has, is suspected of having (or has been diagnosed with) an autoimmune disease, the autoimmune disease may be multiple sclerosis, systemic sclerosis, amyotrophic lateral sclerosis, type 1 diabetes mellitus (T1D), scleroderma, pemphigus or lupus. In embodiments where the subject has, is suspected of having (or has been diagnosed with) an infectious disease, the infectious disease may be HIV, herpes, hepatitis or human papilloma virus. In some embodiments, the infectious disease is HIV. In some preferred embodiments, the infectious disease may be COVID-19 (coronavirus 2019; the disease caused by severe acute respiratory syndrome coronavirus 2, SARS-CoV-2).

The methods of the invention may include isolation and/or expansion steps. For instance, the method may comprise a step of isolating a population of NKT cells from the subject or from a sample derived from the subject. Optionally, the step of isolating may be performed at least 48 hours after glucocorticoid administration; between 48 hours and 13 days after glucocorticoid administration; or between 6 and 48 hours after glucocorticoid administration. In some embodiments (such as embodiments in which the subject has cancer, an infectious disease or microbial disease, or autoimmune disease), the step of isolating the NKT cells may be performed within 3 hours after glucocorticoid administration, and preferably within 1 hour after glucocorticoid administration. The sample may be selected from the group consisting of: blood, plasma, a tumor biopsy or surgically removed tumor, bone marrow, liver, spleen biopsy, and fat or adipose tissue. In some embodiments, the methods further comprise a step of expanding the isolated NKT cells. In some embodiments, the method comprises a step of activating the isolated NKT cells with an NKT cell activator. The NKT cell activator may be a cytokine, a chemokine, a growth factor, and/or an NKT modulating agent such as alpha GalCer (alpha-Galactosylceramide; a-GalCer) sulfatide (3-O-sulfogalactosylceramide; SM4; sulfated galactocerebroside).

The isolated NKT cells of the invention can be further engineered e.g. by transfecting the cells with a nucleic acid. Accordingly, in some embodiments, the method further comprises a step of introducing a nucleic acid encoding a protein into the isolated NKT cells, and culturing the cells under conditions that facilitate expression of said protein. The protein may be one or more of: a T-cell receptor (TCR), a chimeric antigen receptor (CAR), a split, universal and programmable CAR (SUPRA-CAR). The CAR and/or TCR comprises an antigen-binding domain which binds to an antigen selected from the group consisting of: CD19, CD20, CD22, GD2, CD133, EGFR, GPC3, CEA, MUC1, Mesothelin, IL-13R, PSMA, ROR1, CAIX, Her2.

The NKT cells of the invention find uses in medicine. For instance, isolated NKT cells of the invention can be used medically, e.g. in the treatment of cancer, autoimmune disease, or infectious disease (also called microbial disease) in a subject. In these embodiments, the method may comprise administering a therapeutically effective dose of NKT cells isolated via methods disclosed herein, to a subject who suffers one of the aforementioned diseases. In some embodiments, the subject to whom the isolated NKT cells are administered is the same subject from whom the NKT cells were isolated. Alternatively, the subject to whom the isolated NKT cells are administered is different to the subject from whom the NKT cells were isolated.

The NKT cells are administered to the subject by a method selected from the group consisting of: intravenous injection, intraperitoneal injection, intra-lymphatic injection, intrathecal injection, injection into the cerebrospinal fluid (CSF), direct injection into a tumour, and as a gel placed on or near a solid tumour.

This invention also extends to the use of a glucocorticoid in the manufacture of a medicament for use in a method of treatment disclosed herein.

This invention further extends to the use of dexamethasone or other glucocorticoid to induce a population of NKT cells, wherein the population of NKT cells is induced by a method according to any one of statements 101-148.

This invention further extends to the use of dexamethasone or other glucocorticoid to mobilise a population of NKT cells, where in the population of NKT cells are mobilised by a method according to any one of statements 101-148.

Also provided are induced pluripotent stem cells that are derived from the NKT cells of the invention. Thus, in one aspect, the invention provides a method of producing induced pluripotent stem cells (iPSCs), the method comprising reprogramming NKT cells isolated by a method disclosed herein to produce iPSCs. The reprogramming may involve introducing one or more nucleic acids encoding Oct3/4, Klf4, Sox2, and C-myc into the NKT cells. The nucleic acid may be a DNA (e.g. a DNA expression cassette) or an RNA molecule. The reprogramming may further comprise introducing one or more expression cassettes encoding one or more of: Sox1, Sox3, Sox15, Klf1, Klf2, Klf5, L-myc, N-myc, Nanog, and/or LIN28 into the NKT cells. The reprogramming may further comprise introducing one or more of: Sox1, Sox3, Sox15, Klf1, Klf2, Klf5, L-myc, N-myc, Nanog, and/or LIN28 encoding mRNA into the NKT cells. The iPSCs may then be induced to differentiate, e.g. into NKT cells or into an NKT cell lineage.

This invention also provides an isolated natural killer T cell (NKT cell) or a population of natural killer T cells (NKT cell) produced by a method disclosed herein. Relatedly, the NKT cells of the invention may be defined by their expression profile(s), which may be as described elsewhere herein. For instance, the invention provides an isolated natural killer T cell (NKT cell), characterized in that the cell expresses CD3 and optionally expresses one or more of CD4, CD8, CD45, CD49b, CD56, CD62L, NK1.1, Ly6G, Sca1, and/or TCR gamma/delta; and/or does not express: C-kit, B220, FoxP3, and/or TCR alpha/beta. The isolated natural killer T cell (NKT cell) may be from a non-diseased subject.

The NKT cell or its precursor may have been isolated from a subject, wherein the NKT cell or a precursor of the NKT cell was contacted with a high dose glucocorticoid-receptor (GR) modulating agent or ICAM3 modulating agent either in vivo prior to isolation or in vitro after isolation, and wherein the level of CD3 expression is at least two times higher than the average level of CD3 expression in a population of reference NKT cells from the subject. The NKT cell or its precursor may have been isolated from a subject, wherein the NKT cell or a precursor of the NKT cell was contacted with a high dose glucocorticoid-receptor (GR) modulating agent or ICAM3 modulating agent either in vivo prior to isolation or in vitro after isolation, and wherein the level of CD3 expression is at least two times higher than the average level of CD3 expression in a population of reference NKT cells from the subject that have not been contacted with the GR modulating agent or ICAM3 modulating agent.

The CD3 expression levels of said isolated NKT cell and said population of reference NKT cells may be measured by any method known in the art, e.g. by flow cytometry. (The CD3 expression levels of said isolated NKT cell and the CD3 expression levels of said population of reference NKT cells are both to be measured using the same method.) Where flow cytometry is used to measure the expression levels of markers such as CD3, the equipment, reagents, and/or conditions described herein may be used, in conjunction with any methods and protocols known in the art.

The isolated NKT cell of the invention may exhibit CD3 expression levels that are at least three times, at least four times, or at least five times higher than the average level of CD3 expression in a population of reference NKT cells. The population of reference NKT cells may have been obtained from the same subject prior to exposure to the glucocorticoid.

The invention also provides an isolated population of natural killer T cells (NKT cell). The isolated population of NKT cells may be defined by their expression profile(s), which may be as described elsewhere herein. For instance, an isolated population of natural killer T cells (NKT cell) may be characterized in that at least 60, 70, 80, 90, 95, 96, 97, 98, or 99% of the cells express CD3, and/or express one or more of CD4, CD8, CD45, CD49b, CD56, CD62L, NK1.1, Ly6G, Sca1, and TCR gamma/delta; and/or do not express: C-kit, B220, FoxP3, and/or TCR alpha/beta. In humans the NKT cells may express CD56 instead of, or in addition to, CD49b. The NKT cells may not express Sca1. Thus, the NKT cells may express CD3 and/or express one or more of CD4, CD8, CD45, CD49b, CD62L, NK1.1, Ly6G, Sca1, and TCR gamma/delta. The NKT cells may express CD3 and/or express one or more of CD4, CD8, CD45, CD56, CD62L, NK1.1, Ly6G, Sca1, and TCR gamma/delta. The NKT cells may express CD3 and/or express one or more of CD4, CD8, CD45, CD49b, CD62L, NK1.1, Ly6G, and TCR gamma/delta. The NKT cells may express CD3 and/or express one or more of CD4, CD8, CD45, CD56, CD62L, NK1.1, Ly6G, and TCR gamma/delta. The invention provides a glucocorticoid for use in a method of treatment of cancer, autoimmune disease, or infectious disease (also called microbial disease) in a subject, the method comprising administering a glucocorticoid to the subject at a dose equivalent to about 6-45 mg/kg human equivalent dose (HED) of dexamethasone, wherein the glucocorticoid induces/activates/mobilises a population of NKT cells of the invention, as defined herein. For instance, the invention provides a glucocorticoid for use in a method of inducing tumor necrosis, causing NKT tumour infiltration, releasing immune activating cytokines, engulfing and killing tumour cells, promoting infiltration of other immune cells into the tumour, and/or causing CD1d-directed apoptosis in a cancer patient, the method comprising administering a glucocorticoid to the subject at a dose equivalent to about 6-45 mg/kg human equivalent dose (HED) of dexamethasone, to induce a population of NKT cells of the invention, as defined herein. For instance, the invention provides a glucocorticoid for use in a method of inducing tumour necrosis, causing NKT cell tumour infiltration, releasing immune activating cytokines, engulfing and killing tumour cells, promoting infiltration of other immune cells into a tumour, and/or causing CD1d-directed apoptosis in a cancer patient, the method comprising administering a glucocorticoid to the patient at a dose equivalent to about 6-45 mg/kg human equivalent dose (HED) dexamethasone, to mobilise a population of NKT cells of the invention, as defined herein. For instance, the invention provides a glucocorticoid for use in a method of inducing virus death, causing NKT mobilization, releasing immune activating cytokines, engulfing and killing virus-infected cells, promoting infiltration of other immune cells into the virus infected organs, the method comprising administering a glucocorticoid to the subject at a dose equivalent to about 6-45 mg/kg human equivalent dose (HED) of dexamethasone, to induce a population of NKT cells of the invention, as defined herein. The HED of dexamethasone may take any value from the range of values disclosed herein.

### SUMMARY OF THE FIGURES

Embodiments and experiments illustrating the principles of the disclosure will now be discussed with reference to the accompanying figures in which:

FIG. 1. Acute high dose dexamethasone reduces mouse lymphocyte number. Absolute lymphocyte numbers (ALC minus NK and NKT cells) measured by complete blood count (cells/ul=absolute numbers obtained from CBC) 6 hours, 24 hours, 48 hours, 7 days, 13 days, and 21 days after high-dose dexamethasone (18 mg/kg HED Dexamethasone Phosphate (DP)) are significantly reduced as compared to Placebo. At 6 and 48 hours after administration almost complete lymphoablation is observed, with the effect comparable to that achieved with standard Cy/Flu chemotherapy (13 mg/kg HED cyclophosphamide and 0.8 mg/kg HED fludarabine).

FIG. 2. Acute high dose dexamethasone reduces mouse B lymphocyte numbers. B lymphocyte numbers measured by complete blood count (cells/ul=absolute numbers obtained from CBC) 6 hours, 24 hours, 48 hours, 7 days, 13 days, and 21 days after high-dose dexamethasone (18 mg/kg HED DP) are significantly reduced as compared to Placebo. The lymphoablative effect on B lymphocytes is comparable to that achieved with standard Cy/Flu chemotherapy (13 mg/kg HED cyclophosphamide and 0.8 mg/kg HED fludarabine).

FIG. 3. Acute high dose dexamethasone reduces mouse monocyte numbers. Monocyte numbers measured by complete blood count (cells/ul=absolute numbers obtained from CBC) 6 hours, 24 hours, and 48 hours after high-dose dexamethasone (18 mg/kg HED DP) are significantly reduced as compared to Placebo. The ablative effect on monocytes is superior to that achieved by standard Cy/Flu chemotherapy (13 mg/kg HED cyclophosphamide and 0.8 mg/kg HED fludarabine).

Acute high dose dexamethasone reduces mouse neutrophil numbers. Neutrophil numbers measured by complete blood count (cells/ul=absolute numbers obtained from CBC) 6 hours, 24 hours, and 48 hours after high-dose dexamethasone (18 mg/kg HED DP) are significantly reduced as compared to Placebo.

FIG. 5. Acute high dose dexamethasone spares mouse platelets. Acute high dose dexamethasone (18 mg/kg HED DP) does not affect platelet numbers measured by complete blood count (cells/ul=absolute numbers obtained from CBC). Acute high dose dexamethasone therefore eliminates the need for transfusion, and provides a safer, non-toxic alternative to chemotherapeutic regimens.

FIG. 6. Acute high dose dexamethasone spares hematopoietic stem cells. Shown are the number of live hematopoietic stem cells measured at time points between 6 hours and 35 days after treatment of naïve mice with placebo or acute high dose dexamethasone. The acute high dose dexamethasone (18 mg/kg HED DP) does not significantly alter the number of live hematopoietic stem cells. The non-myeloablative regimen represented by acute high dose dexamethasone could, therefore, eliminate the need for transfusions of stem cells for hematopoietic recovery after immune-reset.

FIG. 7. Acute high dose dexamethasone induces NKT upregulation (FIG. 7) and production of a novel population of NKT cells (AVM-NKT). Total NKT cell numbers measured by complete blood count (cells/ul=absolute numbers obtained from CBC) at 6 hours, and 24 hours after high-dose dexamethasone (18 X mg/kg HED DP) are reduced as compared to Placebo. Surprisingly, by 48 hours after high-dose dexamethasone the total NKT cell numbers measured by complete blood count has increased, then reduces gradually until around 13 days after high-dose dexamethasone treatment. With administration of standard Cy/Flu chemotherapy (13 mg/kg HED cyclophosphamide and 0.8 mg/kg HED fludarabine) no such increase in NKT cell numbers is observed at 48 hours after treatment.

FIG. 8. After treatment with high dose dexamethasone, two NKT populations can be identified in peripheral blood. Examination of peripheral blood by flow cytometry after acute high dose dexamethasone identified two NKT cell populations: NKT cells defined as CD3medCD49b+(CD56 in humans), corresponding to previously described NKT cells (central rectangular gate); and, a novel population of NKT cells defined as CD3highCD49b+(CD56 in humans; AVM-NKT cells; center-right rectangular gate). AVM-NKT cells are CD49b+(CD56 in humans) and CD3 very bright, as compared to the known NKT cells which express CD3 with Mean Fluorescent Intensity (MFI) one-half to one log lower than the AVM-NKT.

FIG. 9. Time course of AVM-NKT upregulation. Quantification of the AVM-NKT cells per microliter blood using CBC and flow cytometry results. The AVM-NKT cells are evident in blood of naïve mice between 48 hours-13 days after treatment with one high-dose dexamethasone (HED 18.1 mg/kg DP PO); *=statistically significant.

FIG. 10. Changes in the A20 Tumor Environment induced by treatment with high-dose dexamethasone (HED 18 mg/kg DP). After 48 hours, increased necrosis is evident in tumors of mice treated with high-dose dexamethasone as compared to placebo.

FIG. 11. Acute high dose dexamethasone (AVM0703; HED 18.1 mg/kg PO) significantly delays growth of the A20 B cell lymphoma as compared to placebo. Days of high dose dexamethasone or placebo dosing are indicated by arrows.

FIG. 12. Time course of the percent of typical NKT (non-AVMNKT; left) or AVM-NKT (right) cells from naïve male C57Bl/6 mice after a single oral dose with dexamethasone base at 15 mg/kg measured by flow cytometry that are CD4 positive. Bars show the average for each time point.

FIG. 13. Time course of the percent of typical NKT (non-AVMNKT; left) or AVM-NKT (right) cells from naïve male C57Bl/6 mice after a single oral dose with dexamethasone base at 15 mg/kg measured by flow cytometry that are CD8 positive. Bars show the average for each time point.

FIG. 14. Time course of the percent of typical NKT (non-AVMNKT; left) or AVM-NKT (right) cells from naïve male C57Bl/6 mice after a single oral dose with dexamethasone base at 15 mg/kg measured by flow cytometry that are CD4 CD8 double positive (top left), CD8 single positive (top right), CD4 single positive (bottom left) or CD4 CD8 double negative (bottom right). Bars show the average for each time point.

FIG. 15. Time course of CD3 positive median fluorescence intensity (MFI) (top graph) and arithmetic mean fluorescence intensity (bottom graph) on typical NKT (left) or AVM-NKT (right) cells from naïve male C57Bl/6 mice after a single oral dose with dexamethasone base at 15 mg/kg measured by flow cytometry. Typical NKT cells are CD49b positive (CD56 in humans) with MFI equivalent to AVM-NKT cells. Bars show the average for each time point.

FIG. 16. Time course of CD4 positive median fluorescence intensity (MFI) (top graph) and arithmetic mean fluorescence intensity (bottom graph) on typical NKT (left) or AVM-NKT (right) cells from naïve male C57Bl/6 mice after a single oral dose with dexamethasone base at 15 mg/kg measured by flow cytometry. Typical NKT cells are CD49b positive (CD56 in humans) with MFI equivalent to AVM-NKT cells, but CD3 positive with MFI about 1 log lower than AVM-NKT cells. Bars show the average for each time point

FIG. 17. Time course of CD8 positive median fluorescence intensity (MFI) (top graph) and arithmetic mean fluorescence intensity (bottom graph) on typical NKT (left) or AVM-NKT (right) cells from naïve male C57Bl/6 mice after a single oral dose with dexamethasone base at 15 mg/kg measured by flow cytometry. Typical NKT cells are CD49b positive (CD56 in humans) with MFI equivalent to AVM-NKT cells, but CD3 positive with MFI about 1 log lower than AVM-NKT cells. Bars show the average for each time point.

FIG. 18. Mean Fluorescent Intensity expression of Ly6G on all CD45 dim and positive cells from placebo or 15 mg/kg HED dexamethasone base treated mouse 48 hours after dosing was measured by flow cytometry (MacsQuant, Miltenyi). Dexamethasone treated mice have a population of CD45 dim or positive cells that express Ly6G at much higher levels (MFI about 104) than the majority of CD45 positive cells (MFI about 103). The 104 MFI Ly6G positive cells from the dexamethasone treated mouse also express CD3 very high (MFI about 1 log higher than T lymphocytes and other NKT cells) and are CD49b positive (CD56 in humans) as well.

FIG. 19. AVM NKT express Ly6G, for antitumor, anti-viral, and anti-bacterial responses. The scatter plot shows CD3 fluorescent intensity on the X axis versus Ly6G fluorescent intensity on the Y axis for all CD45 positive cells 48 hrs after AVM0703 HED 18.1 mg/kg dosing. AVM NKT are highlighted in black. Placebo CD3 versus Ly6G scatter is overlaid within the area enclosed in black outline for comparison

FIG. 20. After treatment with high dose dexamethasone, a novel population of CD3 very high T cells can be identified in peripheral blood. Examination of peripheral blood by flow cytometry after acute high dose dexamethasone identified two NKT cell populations: NKT cells defined as CD3medCD49b+, corresponding to previously described NKT cells (central rectangular gate); a novel population of NKT cells defined as CD3highCD49b+(AVM-NKT cells; center-right rectangular gate), as well as a novel CD3 very high T cell (circled in black).

FIG. 21. High dose dexamethasone induces/activates/mobilises a novel population of CD11b very high dendritic cells. The CD11b very high dendritic cells express CD11b about 1 log higher than conventional CD11b+ dendritic cells. High dose dexamethasone also increases the number of conventional CD11b+ dendritic cells.

FIG. 22. AVM0703 (18 mg/kg HED dexamethasone phosphate (DP)) delays A20 B-Lymphoma Growth in a Mouse Model. Shown are the group average tumor volumes over the course of the study. Arrows represent the days the mice were dosed. The graph shows a clear separation of the placebo (n=4) and AVM0703-treated (n=5) mice over the course of the study.

FIG. 23. AVM0703 Delays Endpoint and Eradicates A20 Tumor Cells in the A20 Mouse Lymphoma Model. (A) Images are 2× brightfield microscope images showing that tumor growth in AVM0703 treated mice was pseudogrowth and not true tumor growth as the tumors in the AVM0703-treated mice were largely necrotic, and even in area without full necrosis, no tumor cells were evident. (B) Endpoint curve for the study mice where the x-axis is days from inoculation. The median time to endpoint of the placebo-treated mice was 22 days and the median time to endpoint of the AVM treated mice was 41 days. A Kaplan-Meier analysis determined that the time to endpoint of the AVM treated mice was significantly longer (**p<0.01). (C) Brightfield images of thick sectioned tumors. Visual examination of tumors indicated differences between the placebo and AVM0703 treated mice. Therefore, tumors were thick sectioned and examined under brightfield by microscopy. Tumors from placebo treated mice (left side) were highly cellular with only small areas of necrosis, while tumors from AVM0703 treated mice (right side) were largely necrotic and acellular

FIG. 24. Repeat AVM0703 (18 mg/kg HED dexamethasone phosphate (DP)) Dosing, Up to 7 Doses, Does Not Reduce Body Weight. Graph of average body weight for each mouse group (n=4 placebo, n=5 AVM0703) over the course of the study. Arrows below the x-axis indicate the days of dosing. The dotted line represents 20% loss of the average body weight of all mice at the start of the study. One mouse reached the threshold after 8 doses and was euthanized.

FIG. 25. Organ Weight to Body Weight Ratio at Study Endpoint. Graphs of organ weight to body weight ratio. Colon weights were significantly higher in AVM0703 treated mice (n=5) compared to placebo (n=4); however, this may be due to the significant increase in age of AVM0703 treated mice at euthanization (between 14 to 40 days older than placebo mice at euthanization). *p<0.1.

FIG. 26. Duration of Responsiveness to Repeat AVM0703 Determined by Reductions in Spleen and Thymus Weights BALB/c mice were randomized into 2 groups and treated orally with 18.06 mg/kg AVM0703 HED DP (n=5) or placebo (n=4). Graph of spleen (left) and thymus (right) weight to body weight ratio based on the number of days since the last dose of AVM0703. The number near each dot represents the total number of AVM0703 doses received before the mouse reached study endpoint. The dotted line represents the average spleen or thymus to body weight ratio following placebo treatment. AVM0703 continues to affect both the thymus and the spleen up to 7 doses, where spleen and thymus weight is reduced compared to placebo on Days 1 and 3 post dose and has almost returned to placebo values by 6 days after the 7th dose. AVM0703 reduction in spleen and thymus weight appears to be lost after 8 doses. Avg.=average; DP=dexamethasone phosphate; HED=human equivalent dose.

FIG. 27. Tumors Stained With Hematoxylin and Eosin and Imaged at 20× Magnification. Stars indicate areas of necrosis. Black arrows indicate areas of neoplastic growth extending in the direction of the arrows. A. 0 mg/kg DP; B. 7 mg/kg DP; C. 18 mg/kg DP; E. 25 mg/kg DP; red areas indicate hemorrhaging; E. Mean pathology scores averaged across tumors where n=2 tumors, DP=dexamethasone phosphate.

FIG. 28. Tumor CD3 Expression. Images of tumors stained via immunohistochemistry for CD3 and imaged at 100× magnification. Black arrows indicate infiltration of CD3+ round cells in the direction of the arrows. ‘N’ indicates areas of neoplastic growth. A.0 mg/kg DP, ‘By’ indicates blood vessels; B. 7 mg/kg DP; C. 18 mg/kg DP; D. 25 mg/kg DP. DP=dexamethasone phosphate.

FIG. 29. Tumor NKp46 Expression. Images of tumors stained via immunohistochemistry for NK cell marker NKp46 and imaged at 100× magnification. Stars indicate areas of necrosis. Black arrows indicate examples of cells positive for NKp46. ‘N’ indicates areas of neoplastic growth. A. 0 mg/kg DP; B. 7 mg/kg DP; C. 18 mg/kg DP; D. 25 mg/kg DP. DP=dexamethasone phosphate; NK=natural killer.

FIG. 30. Tumor CD49b Expression Images of tumors stained via immunohistochemistry for CD49b and imaged at 100× magnification. Black arrows indicate examples of cells positive for CD49b. Blue arrows indicate blood vessels or endothelial cells labeled with CD49b. ‘N’ indicates areas of neoplastic growth. A. 0 mg/kg DP; B. 7 mg/kg DP; C. 18 mg/kg DP; D. 25 mg/kg DP. DP=dexamethasone phosphate.

FIG. 31. Tumor Apoptosis. Images of tumors stained via immunohistochemistry for apoptosis marker caspase 3 and imaged at 40× magnification. Stars indicate areas of necrosis. Black arrows indicate examples of cells positive for caspase 3. ‘N’ indicates areas of neoplastic growth. A. 0 mg/kg DP; B. 7 mg/kg DP; C. 18 mg/kg DP; D. 25 mg/kg DP. DP=dexamethasone phosphate.

FIG. 32. Resorbed Tumor From a Mouse Treated With AVM0703 and Cy/Flu Combination.

FIG. 33. Tumor Examples From Study AVM_CANMOD_05—Lymphodepletion Subset. Left: Placebo tumor example; 956 mm³, L 15.06, W 11.27 mm, 0.54 g; Right: AVM0703 (25 mg/kg) tumor example; 203.25 mm³, L 7.67, W 7.28 mm, 0.086 g.

FIG. 34. Tumor Examples From Study AVM_CANMOD_05—Endpoint Analysis Subset. Left: Placebo tumor example. Right: 18 mg/kg Mouse 11 AVM0703 tumor example.

FIG. 35. Evidence of Tumor Lysis Syndrome in a CCRF CEM tumor bearing mouse treated with AVM0703. Tumor lysis is indicated by a substantial greenish, oily area within the tumor.

FIG. 36. Graph of tumor volumes for mice inoculated with CCRF-CEM cells and treated with either placebo (n=2) or 18 mg/kg AVM0703 (n=3). Mice were dosed once weekly beginning on Day 7 post-inoculation, and dosing events are marked with black arrows. Mice were euthanized if tumor volume exceeded 1500 mm³.

FIG. 37. Endpoint curve of mice inoculated with CCRF-CEM cells. Mice were euthanized when tumor volume reached endpoint at 1500 mm³. Both placebo and AVM0703 treated groups had an n=2.

FIG. 38. AVM0703 induces long term immunity against human T-ALL xenograft in NCR nude mice. An AVM0703 treated mouse was re-challenged with human T-ALL (CCRF-CEM cell line) on day 118, with no tumour growth observed out to day 164, indicating that AVM0703 triggers long-term immunity.

FIG. 39. CD45/CD56 scattergrams from an osteoarthritis patient treated with 3-6 mg/kg DSP. AVM-NKT cells (indicated by a rectangular box) were identified and like in mice are CD45 dim and CD56 very bright (CD49b in mice).

FIG. 40. Flow cytometry data from a healthy blood donor and prostate cancer patient 1 hour and 3 hours after administration of 6 mg/kg AVM0703. In the prostate cancer patient a novel CD45dim CD56bright cell population (circled) is evident 1 hour after infusion. These data indicate that human patients mobilise cells corresponding to the AVM-NKT cells identified in mice.

### DETAILED DESCRIPTION

The present disclosure pertains to: methods of producing/activating/mobilising a population of natural killer T cells (NKT cells), isolated NKT cells or isolated populations of NKT cells produced by such methods, and methods of treatment in which NKT cells are induced in a subject, or are administered to a subject; methods of producing/activating/mobilising a population of T cells, isolated T cells or isolated populations of T cells produced by such methods, and methods of treatment in which T cells are induced in a subject, or are administered to a subject; methods of producing/activating/mobilising a population of dendritic cells, isolated dendritic cells or isolated populations of dendritic cells produced by such methods, and methods of treatment in which dendritic cells are induced in a subject, or are administered to a subject; and, methods of activating a population of dendritic cells, isolated dendritic cells or isolated populations of dendritic cells produced by such methods, and methods of treatment in which dendritic cells are induced in a subject, or are administered to a subject.

In some embodiments, the disclosed methods are methods of producing populations of natural killer T cells (NKT cells) and T cells, and activating a population of dendritic cells. In other embodiments, the disclosed methods are methods of mobilising populations of natural killer T cells (NKT cells), T cells, and/or dendritic cells. As used herein, to “mobilize” such cells can mean to promote movement of these out of lymphoid organs/tissues (for example, the thymus and spleen) and into the systemic circulation (where they may then move to other sites, e.g. tumour sites). The disclosed methods may include multiple of the above aspects. For example a method of the disclosure may both induce production of a population of NKT cells as described herein and mobilise a population of NKT cells as described herein. For example, a method of the disclosure may induce production of a population of NKT cells as described herein in the thymus and/or spleen and/or bone marrow, and mobilise a population of NKT cells as described herein from the thymus and/or spleen and/or bone marrow.

As disclosed herein, the methods of producing a population of natural killer T cells (NKT cells), producing a population of T cells, and/or producing or activating a population of dendritic cells comprise administering to a subject a glucocorticoid-receptor (GR) modulating agent or ICAM3 modulating agent. The glucocorticoid-receptor (GR) modulating agent or ICAM3 modulating agent induces the population of NKT cells, induces the population of T cells, and/or activates the population of dendritic cells in the subject. The glucocorticoid-receptor (GR) modulating agent or ICAM3 modulating agent may mobilize the population of NKT cells, mobilize the population of T cells, and/or activate or mobilize the population of dendritic cells in the subject

Also disclosed are isolated populations of NKT cells, isolated NKT cells, isolated populations of T cells, isolated T cells, isolated populations of dendritic cells, and isolated dendritic cells which may be produced by the disclosed methods.

The disclosed NKT cells may be characterized by the pattern of surface proteins which they express. In some embodiments, the disclosed NKT cells may express CD3, CD4, CD8, CD45, CD49b, CD56, CD62L, NK1.1, Ly6G, Sca1, and/or TCR gamma/delta. In some embodiments, the disclosed NKT cells may not express C-kit, B220, FoxP3, and/or TCR alpha/beta. In humans the NKT cells may express CD56 instead of, or in addition to, CD49b. In some embodiments the NKT cells do not express Sca1. Thus, the disclosed NKT cells may express CD3, CD4, CD8, CD45, CD56, CD62L, NK1.1, Ly6G, Sca1, and/or TCR gamma/delta. The disclosed NKT cells may express CD3, CD4, CD8, CD45, CD49b, CD62L, NK1.1, Ly6G, Sca1, and/or TCR gamma/delta. The disclosed NKT cells may express CD3, CD4, CD8, CD45, CD56, CD62L, NK1.1, Ly6G, and/or TCR gamma/delta. The disclosed NKT cells may express CD3, CD4, CD8, CD45, CD49b, CD62L, NK1.1, Ly6G, and/or TCR gamma/delta. In some embodiments, the disclosed NKT cells may or may not express CD44, CD69, and/or CD25. In some embodiments the disclosed NKT cells may express CD56.

In embodiments relating to populations of the disclosed NKT cells, the population of NKT cells may be characterized in that at least 60, 70, 80, 90, 95, 96, 97, 98, or 99% of the NKT cells express CD3, CD4, CD8, CD45, CD49b, CD56, CD62L, NK1.1, Ly6G, Sca1, and/or TCR gamma/delta. The NKT cells may express CD3, CD4, CD8, CD45, CD49b, CD62L, NK1.1, Ly6G, Sca1, and/or TCR gamma/delta. The NKT cells express CD3, CD4, CD8, CD45, CD56, CD62L, NK1.1, Ly6G, Sca1, and/or TCR gamma/delta. The NKT cells may express CD3, CD4, CD8, CD45, CD49b, CD62L, NK1.1, Ly6G, and/or TCR gamma/delta. The NKT cells express CD3, CD4, CD8, CD45, CD56, CD62L, NK1.1, Ly6G, and/or TCR gamma/delta. In some such embodiments, the population of NKT cells may be characterized in that at least 60, 70, 80, 90, 95, 96, 97, 98, or 99% of the NKT cells do not express: C-kit, B220, FoxP3, and/or TCR alpha/beta. In some embodiments the population of NKT cells may be characterized in that at least 60, 70, 80, 90, 95, 96, 97, 98, or 99% of the NKT cells express CD56.

The disclosed T cells may be characterized by the pattern of surface proteins which they express. The disclosed T cells express CD3 at very high MFI. In embodiments relating to populations of the disclosed T cells, the population of T cells may be characterized in that at least 60, 70, 80, 90, 95, 96, 97, 98, or 99% of the T cells express CD3. In some embodiments, the disclosed T cells may express CD3, CD4, CD45, and/or CD49b (CD56 in humans). In some embodiments, the disclosed T cells may express TCR gamma/delta. In some embodiments, the disclosed T cells may express TCR alpha/beta. In some embodiments, the disclosed T cells may express CD8. In some embodiments, the disclosed T cells may not express CD8. In embodiments relating to populations of the disclosed T cells, the population of T cells may be characterized in that at least 60, 70, 80, 90, 95, 96, 97, 98, or 99% of the NKT cells express CD3, CD4, CD45, and/or CD49b (CD56 in humans). In some embodiments relating to populations of the disclosed T cells, the population of T cells may be characterized in that at least 60, 70, 80, 90, 95, 96, 97, 98, or 99% of the T cells express TCR gamma/delta. In some embodiments relating to populations of the disclosed T cells, the population of T cells may be characterized in that at least 60, 70, 80, 90, 95, 96, 97, 98, or 99% of the T cells express TCR alpha/beta. In some such embodiments, the population of T cells may be characterized in that at least 60, 70, 80, 90, 95, 96, 97, 98, or 99% of the T cells express CD8. In some such embodiments, the population of T cells may be characterized in that at least 60, 70, 80, 90, 95, 96, 97, 98, or 99% of the T cells do not express CD8. In preferred embodiments the T cells or populations of T cells express CD8 and/or express TCR gamma/delta.

The disclosed dendritic cells may be characterized by the pattern of surface proteins which they express. The disclosed dendritic cells express CD11b. In embodiments relating to populations of the disclosed dendritic cells, the population of dendritic cells may be characterized in that at least 60, 70, 80, 90, 95, 96, 97, 98, or 99% of the dendritic cells express CD11b.

Expression of surface proteins on cells can be readily determined using techniques well-known to the skilled person—for example, enzyme-linked immunosorbent assays (ELISA), magnetic-activated cell sorting (MACS), or flow cytometry techniques. Flow cytometry uses the light properties scattered from cells bound by fluorescently-tagged antibodies to identify cells expressing surface proteins of interest. Flow cytometry can determine not only whether a cell is expressing a protein of interest but can also indicate the amount of protein expressed by cells on the basis of intensity of fluorescence. In flow cytometric readouts, and as used herein: “+” (or “positive”) indicates expression of a given surface protein; “−” (or “negative”) indicates no expression of a given surface protein; and “+/−” indicates bimodal expression of a given surface protein. Expressions such as “bright” (sometimes “high” or “++”), “dim” (sometimes “low”), and “moderate” are used to indicate the relative amount of a particular cell surface protein.

### CD3

CD3 (cluster of differentiation 3) is a T-cell co-receptor, which helps to activate cytotoxic T cells (CD8+ naive T cells) and T helper cells (CD4+ naive T cells). Because CD3 is required for T cell activation, drugs (e.g. monoclonal antibodies) that target it are being investigated as immunosuppressant therapies (e.g. otelixizumab) for type 1 diabetes and other autoimmune diseases. Known NKT cells described in the literature express CD3 with mean fluorescent intensity (MFI) about 1 log lower than the NKT cells of the present disclosure. Similarly, known T cells and NKT cells described in the literature express CD3 with mean fluorescent intensity (MFI) about 1-1.5 log lower than the T cells of the present disclosure.

In some embodiments, the NKT cells of the disclosure express CD3. In some embodiments, the NKT cells of the disclosure are CD3+/very bright. In embodiments relating to populations of the NKT cells of the disclosure, at least 5, 10, 20, 30, 40, 50, 60, 70, 80, 90, 95, 96, 97, 98, or 99% of the NKT cells may express CD3. In some embodiments at least 5, 10, 20, 30, 40, 50, 60, 70, 80, 90, 95, 96, 97, 98, or 99% of the NKT cells may be CD3+/very bright.

The T cells of the disclosure are CD3+/very bright. In embodiments relating to populations of the T cells of the disclosure, at least 5, 10, 20, 30, 40, 50, 60, 70, 80, 90, 95, 96, 97, 98, or 99% of the T cells may be CD3+/very bright.

### CD4

CD4 (cluster of differentiation 4) is a glycoprotein found on the surface of immune cells including T-helper cells and monocytes. CD4 is a co-receptor of the T cell receptor (TCR), which it assists in communicating with antigen presenting cells for antigen-induced T cell activation. Cross-linking of CD4 can induce T cell apoptosis via the Fas Ligand pathway.

In some embodiments, the NKT cells of the disclosure express CD4. In some embodiments, the NKT cells of the disclosure are CD4+/very bright. In embodiments relating to populations of the NKT cells of the disclosure, at least 5, 10, 20, 30, 40, 50, 60, 70, 80, 90, 95, 96, 97, 98, or 99% of the NKT cells may express CD4. In some embodiments at least 5, 10, 20, 30, 40, 50, 60, 70, 80, 90, 95, 96, 97, 98, or 99% of the NKT cells may be CD4+/very bright.

In some embodiments, the T cells of the disclosure express CD4. In embodiments relating to populations of the T cells of the disclosure, at least 5, 10, 20, 30, 40, 50, 60, 70, 80, 90, 95, 96, 97, 98, or 99% of the T cells may express CD4.

### CD8

CD8 (cluster of differentiation 8) is a transmembrane glycoprotein that serves as a co-receptor for the T cell receptor (TCR). It is predominantly expressed on the surface of cytotoxic T cells, but is also expressed on natural killer cells. On T cells it plays roles in T cell—antigen interaction and T cell signalling.

In some embodiments, the NKT cells of the disclosure express CD8. In some embodiments, the NKT cells of the disclosure are CD8+/dim. In embodiments relating to populations of the NKT cells of the disclosure, at least 5, 10, 20, 30, 40, 50, 60, 70, 80, 90, 95, 96, 97, 98, or 99% of the NKT cells may express CD8. In some embodiments at least 5, 10, 20, 30, 40, 50, 60, 70, 80, 90, 95, 96, 97, 98, or 99% of the NKT cells may be CD8+/dim. In some embodiments, the disclosed NKT cells may not express CD8.

In some preferred embodiments, the NKT cells of the disclosure express CD4 and CD8. In some preferred embodiments relating to populations of the NKT cells of the disclosure, at least 5, 10, 20, 30, 40, 50, 60, 70, 80, 90, 95, 96, 97, 98, or 99% of the NKT cells may express CD4 and CD8. In some embodiments the NKT cells of the disclosure are not CD4 and CD8 double negative. In some embodiments relating to populations of the NKT cells of the disclosure, none of the NKT cells are CD4 and CD8 double negative.

In some embodiments, the T cells of the disclosure may not express CD8. In embodiments relating to populations of the T cells of the disclosure, at least 5, 10, 20, 30, 40, 50, 60, 70, 80, 90, 95, 96, 97, 98, or 99% of the T cells may not express CD8.

### CD45

CD45 (cluster of differentiation 45; also known as Protein tyrosine phosphatase, receptor type; PTPRC) is an essential regulator of T- and B-cell antigen receptor signalling, and a marker for all white blood cells. CD45 expression is essential for T cell activation by the TCR. CD45 may be a receptor for CD26.

In some embodiments, the NKT cells of the disclosure express CD45. In some embodiments, the, the NKT cells of the disclosure are CD45+/dim. In embodiments relating to populations of the NKT cells of the disclosure, at least 5, 10, 20, 30, 40, 50, 60, 70, 80, 90, 95, 96, 97, 98, or 99% of the NKT cells may express CD45. In some embodiments at least 5, 10, 20, 30, 40, 50, 60, 70, 80, 90, 95, 96, 97, 98, or 99% of the NKT cells may be CD45+/dim.

In some embodiments, the T cells of the disclosure may express CD45. In some embodiments, the T cells of the disclosure are CD45+/dim. In embodiments relating to populations of the T cells of the disclosure, at least 5, 10, 20, 30, 40, 50, 60, 70, 80, 90, 95, 96, 97, 98, or 99% of the T cells may express CD45. In some embodiments at least 5, 10, 20, 30, 40, 50, 60, 70, 80, 90, 95, 96, 97, 98, or 99% of the T cells may be CD45+/dim.

The CD45 may be any isoform of CD45, such as CD45RA, CD45RO and/or CD45RABC (also known as CD45R; also known as B220).

### CD49b

CD49b (cluster of differentiation 49b; also known as integrin alpha-2) is an integrin alpha subunit. It makes up half of the α2β1 integrin duplex. CD49b is used as a marker of Natural Killer (NK) cells; the cytotoxicity of NK cells expressing CD49b is known to be much greater than that of NK cells that do not express CD49b.

In some embodiments, the NKT cells of the disclosure express CD49b. In embodiments relating to populations of the NKT cells of the disclosure, at least 5, 10, 20, 30, 40, 50, 60, 70, 80, 90, 95, 96, 97, 98, or 99% of the NKT cells may express CD49b.

In some embodiments, the T cells of the disclosure may express CD49b. In embodiments relating to populations of the T cells of the disclosure, at least 5, 10, 20, 30, 40, 50, 60, 70, 80, 90, 95, 96, 97, 98, or 99% of the T cells may express CD49b.

### CD56

CD56 (cluster of differentiation 56; also known as neural cell adhesion molecule, NCAM) is a homophilic binding glycoprotein expressed on the surface of neurons, glia and skeletal muscle. CD56 expression is associated with natural killer cells.

In some embodiments, the NKT cells of the disclosure express CD56. In embodiments relating to populations of the NKT cells of the disclosure, at least 5, 10, 20, 30, 40, 50, 60, 70, 80, 90, 95, 96, 97, 98, or 99% of the NKT cells may express CD56.

In some embodiments, the NKT cells of the disclosure are CD56+/bright. In embodiments relating to populations of the NKT cells of the disclosure, at least 5, 10, 20, 30, 40, 50, 60, 70, 80, 90, 95, 96, 97, 98, or 99% of the NKT cells may be CD56+/bright.

### CD62L

CD62L (cluster of differentiation 62L; also known as L-selectin) is a marker of cell activation. CD62L is also called L-selectin and can mediate cell-cell adhesion initiating the process of cells moving across the endothelium out of the blood and into tissues and organs

In some embodiments, the NKT cells of the disclosure express CD62L. In embodiments relating to populations of the NKT cells of the disclosure, at least 5, 10, 20, 30, 40, 50, 60, 70, 80, 90, 95, 96, 97, 98, or 99% of the NKT cells may express CD62L.

### NK1.1

NK1.1 (also known as: killer cell lectin-like receptor subfamily B, member 1; KLRB1; NKR-P1A; CD161 (cluster of differentiation 161)) is a marker of mature NK cells; its activation induces NK cells to kill otherwise insensitive targets, and may also induce NK cells to proliferate. NKT cells were first observed as a population of T lymphocytes expressing this pan-NK cell marker.

In some embodiments, the NKT cells of the disclosure express NK1.1. In embodiments relating to populations of the NKT cells of the disclosure, at least 5, 10, 20, 30, 40, 50, 60, 70, 80, 90, 95, 96, 97, 98, or 99% of the NKT cells may express NK1.1.

### Ly6G

Ly6G (lymphocyte antigen 6 complex locus G6D) is a marker for fully mature and differentiated neutrophils or granulocytes, and has also been implicated in antitumor responses. Ly6G is usually a marker for monocytes and neutrophils and granulocytes, indicating that the NKT cells of the disclosure are distinct from known NKT cells, and may not only be able to directly kill cancer cells that express CD1d, as well as activate other NK cells and B and T lymphocytes and secrete cytokines, but may also be able to engulf cancer cells and pathogens directly.

In some embodiments, the NKT cells of the disclosure express Ly6G. In embodiments relating to populations of the NKT cells of the disclosure, at least 5, 10, 20, 30, 40, 50, 60, 70, 80, 90, 95, 96, 97, 98, or 99% of the NKT cells may express Ly6G.

### CD1

CD1 molecules are lipid-presenting glycoproteins. Humans express five CD1 proteins (CD1a-e), four of which (CD1a-d) are trafficked to the cell surface, where they may display lipid antigens to T-cell receptors. This interaction may lead to both non-cognate and cognate T cell help to B cells, the latter eliciting anti-lipid antibody response. All CD1 proteins can bind a broad range of structurally different exogenous and endogenous lipids, but each shows a preference to one or more lipid classes (Kaczmarek et al, 2017, which is hereby incorporated by reference in its entirety). This unorthodox binding behavior is the result of elaborate architectures of CD1 binding clefts and distinct intracellular trafficking routes. Together, these features make CD1 system a versatile player in immune response, sitting at the crossroads of innate and adaptive immunity. While CD1 system may be involved in numerous infectious, inflammatory, and autoimmune diseases, its involvement may lead to opposite outcomes depending on different pathologies (Kaczmarek et al, 2017).

### CD11b

CD11b (Cluster of differentiation molecule 11b, also known as CR3a and Integrin alpha M, ITGAM) is an integrin family member which pairs with CD18 to form the CR3 heterodimer. CD11b is expressed on the surface of many leukocytes including monocytes, neutrophils, natural killer cells, granulocytes and macrophages. Known dendritic cells described in the literature express CD11b with mean fluorescent intensity (MFI) about 1 log lower than the dendritic cells of the present disclosure. The dendritic cells of the disclosure are CD11b+/very bright. In embodiments relating to populations of the dendritic cells of the disclosure, at least 5, 10, 20, 30, 40, 50, 60, 70, 80, 90, 95, 96, 97, 98, or 99% of the dendritic cells may be CD11b+/very bright.

### Major Histocompatability Complex; MHC

The MHC was discovered by Gorer and Snell et al in 1936. Their skin transplantation experiments with mice revealed that self- and non-self recognition depended on the genetic background. Snell et al named the group of mouse genes that determine self/non-self as histocompatibility-2 (H-2). The genomic loci of the MHC encode polymorphic cell-membrane-bound glycoproteins known as MHC classical class I and class II molecules (antigens), which regulate the immune response by presenting peptides of fragmented proteins to circulating cytotoxic and helper T lymphocytes, respectively. Classical MHC class 1 proteins have been subdivided as HLA-A, HLA-B and HLA-C(Nakamura et al, 2019, which is hereby incorporated by reference in its entirety). On the other hand, HLA-E, HLA-F, HLA-G, MHC class I polypeptide-related sequence A (MICA) and FcRn etc. are classified as non-classical MHC class I.

MHC classical class I molecules are expressed in most tissues and they associate non-covalently with b2-microglobulin to present intracellularly processed peptide antigens (which are 8-11 amino acids in length) to T-cell receptors of specific CD8+ T cells in order to induce their activation and/or cytotoxicity (Shiina et al 2016, which is hereby incorporated by reference in its entirety). The processed peptides may arise from the cell's own proteome or from foreign intracellular pathogens. Mature dendritic cells use the MHC class I system to present peptides deriving from antigens captured by endocytosis. This process, called cross-presentation, plays a crucial role in the initiation of responses of specific T CD8+ lymphocytes in peripheral lymphoid organs (Shiina et al). In addition, the MHC classical class I proteins may act as ligands for killer-cell immunoglobulin-like receptors that regulate the cytotoxic activity of cytotoxic T cells and natural killer cell and leucocyte immunoglobulin-like receptors expressed on myelomonocytes and other leucocyte lineages. In contrast to the classical class I antigens, the classical class II antigens form heterodimeric structures specialized in the presentation of exogenous peptides (15-25 amino acids in length) on the surface of lymphoid cells to the CD4+ helper T lymphocytes of the immune system. The class II gene expression is predominantly restricted to the lymphoid cells, such as B cells, monocytes, macrophages, endothelial cells, dendritic cells and activated T cells. MHC class II proteins are identified as HLA-DR, HLA-DP and HLA-DQ. The MHC class II genes include HLA-DRA1, HLA-DQA1, HLA-DPA1 encoding a chain, HLA-DRB1, HLA-DRB3, HLA-DRB4, HLA-DRB5 (HLA-DRB3/4/5), HLA-DQB1, and HLA-DPB1 encoding β chain. HLA-DRA1 forms a heterodimer with HLA-DRB1 or HLA-DRB3/4/5 (Nakamura et al). Similarly, HLA-DQA1 and HLA-DPA1 are also associated with HLA-DQB1 and HLA-DPB1, respectively. The HLA-DR is divided into 5 groups consisting of DR1, DR51, DR52, DR53 and DR8 depending on the antigen group. The DR1 and DR8 groups both consist only of DRB1 as an expressed gene. On the other hand, The DR51, DR52, and DR53 groups contain DRB1 in common and furthermore consist of DRB5, DRB3, and DRB4, which is considered to be generated from DRB1 gene by gene duplication, as expressed genes, respectively (Nakamura et al).

Both the classical class I and class II genes are often highly polymorphic, presumably to preserve the inter-individual variability of the antigen-presenting ability and help the species to defend against and survive the natural selection pressure from various infectious agents. The non-classical class I and class II antigens, although similar in structure to their classical class I or class II counterparts, are usually far less polymorphic, have variable or limited tissue expression and functions that are often distinctly different to those of the classical class I or class II antigens. Moreover, several non-classical MEW class I genes are located outside the MHC (Shiina et al).

The loci of the HLA complex (such as HLA-A, HLA-B, HLA-C, HLA-DR, HLA-DQ, and HLA-DP) have many polymorphisms, so the combination (haplotype) is exceedingly large. However, the MHC exhibits strong linkage disequilibrium, which is the appearance of non-random association of alleles at multiple loci. This linkage disequilibrium in the MHC region often causes a specific combination for each locus of MHC. When two genetic polymorphisms are present on the same chromosome, the two polymorphisms are classified as linked (Nakamura et al). Given that genetic recombination has occurred in a biologically conventional manner, polymorphisms at separate sites are not able to be determined as in the linked state. However, linkage disequilibrium is a state where certain gene polymorphism can be predicted with extremely high probability based on information of the polymorphism at a distant site. In the MHC, the gene loci are concentrated in a narrow region of chromosome 6, so recombination between each gene is less likely to occur. Therefore, genes such as HLA-A, HLA-B, HLA-C, and HLA-DRB1 are often inherited in a linkage disequilibrium state. As HLA gene polymorphism analysis progresses, haplotypes that are associated with specific diseases that that are frequently found in specific ethnic groups have been elucidated. These ethnic group-specific haplotypes are thought to be involved in the process of forming ethnic groups. Thus, these haplotypes are commonly used to search for ethnic roots.

In humans, the MHC classical class I genes are involved critically in organ transplant rejection and graftversus-host disease following haematopoietic stem cell transplants. Various associations have been evidenced between HLA class I molecules and the numerous autoimmune diseases, as well as infectious diseases and drug adverse reactions. Apart from their essential role in the elaboration of adaptive immune responses, the role of MHC class I genes was demonstrated in various steps of reproduction such as pregnancy maintenance, mate selection and kin recognition. The MHC has also been considered to be a system primarily for sexual selection and avoidance of inbreeding with histocompatibility fulfilling a secondary role. The MHC class I gene products also have impact on central nervous system development and plasticity, neurological cell interactions, synaptic function and behaviour, cerebral hemispheric specialization, and neurological and psychiatric disorders. Hence, the human MHC class I region is one of the most biomedically diverse and important genomic regions (Shiina et al).

### TCR Gamma/Delta

T-cell receptor gamma delta (TCR gamma/delta; TCR γδ) is a T-cell receptor that is made up of one γ (gamma) chain and one δ (delta) chain. TCR gamma/delta expressing T-cells (gamma delta T cells) are important recognizers of lipid antigens expressed by cancer cells as well as stressed cells such as cancer cells, microbial and viral infected cells and autoreactive lymphocytes. Gamma delta T cells exhibit several characteristics that place them at the border between the more evolutionarily primitive innate immune system that permits a rapid beneficial response to a variety of foreign agents and the adaptive immune system, where B and T cells coordinate a slower but highly antigen-specific immune response leading to long-lasting memory against subsequent challenges by the same antigen. Gamma delta T cells may be considered a component of adaptive immunity in that they rearrange TCR genes to produce junctional diversity and can develop a memory phenotype.

The most common human gamma delta variant is the Vgamma9/Vdelta2 variant in blood, while Vdelta1 type gamma delta T cells in tumors have been associated with prognosis. A Vdelta3 variant has also been described, as has a Vdelta2 negative variant following CMV infection which reduced cancer risk. In contrast to MHC-restricted alpha beta T cells, gamma delta T cells do not require antigen processing and MHC presentation of peptide epitopes, although some can recognize MHC class lb. Consequently, tumor cells cannot evade detection by down-regulating MHC and gamma delta T cells thus also have equal potential for killing tumors with low mutational load, and are less likely to be affected by resistance issues. Gamma delta T cell tumor infiltration has also been correlated highest with survival and lower incidence of graft versus host disease. Gamma delta T cells naturally home to various tissues to detect tumors and are preferred for allogeneic therapy over alpha beta T cells.

In some embodiments, the NKT cells of the disclosure express TCR gamma/delta. In embodiments relating to populations of the NKT cells of the disclosure, at least 5, 10, 20, 30, 40, 50, 60, 70, 80, 90, 95, 96, 97, 98, or 99% of the NKT cells may express TCR gamma/delta.

In some preferred embodiments, the NKT cells of the disclosure express Ly6G and TCR gamma/delta. In some preferred embodiments relating to populations of the NKT cells of the disclosure, at least 5, 10, 20, 30, 40, 50, 60, 70, 80, 90, 95, 96, 97, 98, or 99% of the NKT cells may express Ly6G and TCR gamma/delta. The expression of both Ly6G and TCR gamma delta suggests that the NKT cells of the disclosure, in addition to having functions of known NKT cells, may also directly engulf cancer cells or pathogens.

In some embodiments, the T cells of the disclosure may express TCR gamma/delta. In embodiments relating to populations of the T cells of the disclosure, at least 5, 10, 20, 30, 40, 50, 60, 70, 80, 90, 95, 96, 97, 98, or 99% of the T cells may express TCR gamma/delta.

### Sca1

Sca1 (stem cell antigen-1; also known as Ly6A) is the common biological marker used to identify hematopoietic stem cell (HSC) along with other markers. Sca-1 plays a role in hematopoietic progenitor/stem cell lineage fate and C-kit expression. Its bright expression on the NKT cells of the disclosure may indicate that these are activated memory stem cells.

In some embodiments, the NKT cells of the disclosure express Sca1. In some embodiments, the NKT cells of the disclosure are Sca1+/very bright. In embodiments relating to populations of the NKT cells of the disclosure, at least 5, 10, 20, 30, 40, 50, 60, 70, 80, 90, 95, 96, 97, 98, or 99% of the NKT cells may express Sca1. In some embodiments at least 5, 10, 20, 30, 40, 50, 60, 70, 80, 90, 95, 96, 97, 98, or 99% of the NKT cells may be Sca1+/very bright.

### C-Kit

C-kit (also known: as tyrosine-protein kinase KIT; CD117 (cluster of differentiation 117); mast/stem cell growth factor receptor (SCFR)) is a receptor tyrosine kinase protein, expressed on the surface of hematopoietic stem cells. That the NKT cells of the disclosure do not express C-kit indicates that they are not hematopoietic stem cells.

In some embodiments, the NKT cells of the disclosure may not express C-kit. In embodiments relating to populations of the NKT cells of the disclosure, at least 5, 10, 20, 30, 40, 50, 60, 70, 80, 90, 95, 96, 97, 98, or 99% of the NKT cells may not express C-kit.

### B220

B220 (which is an isoform of CD45) is a marker for B cells.

In some embodiments, the NKT cells of the disclosure may not express B220. In embodiments relating to populations of the NKT cells of the disclosure, at least 5, 10, 20, 30, 40, 50, 60, 70, 80, 90, 95, 96, 97, 98, or 99% of the NKT cells may not express B220.

### FoxP3

FoxP3 (forkhead box P3; also known as scurfin) is a member of the forkhead box protein family, and is believed to function as a master regulator of the regulatory pathway in the development and function of regulatory T cells. That the NKT cells of the disclosure do not express FoxP3 indicates that they are not regulatory cells, and therefore should not dampen the immune response to cancer or a pathogen.

In some embodiments, the NKT cells of the disclosure may not express FoxP3. In embodiments relating to populations of the NKT cells of the disclosure, at least 5, 10, 20, 30, 40, 50, 60, 70, 80, 90, 95, 96, 97, 98, or 99% of the NKT cells may not express FoxP3.

### TCR Alpha/Beta

T-cell receptor alpha beta (TCR alpha/beta; TCR αβ) is the predominant TCR heterodimer that is made up of one α (alpha) chain and one β (beta) chain.

In some embodiments, the NKT cells of the disclosure may not express TCR alpha/beta. In embodiments relating to populations of the NKT cells of the disclosure, at least 5, 10, 20, 30, 40, 50, 60, 70, 80, 90, 95, 96, 97, 98, or 99% of the NKT cells may not express TCR alpha/beta.

In some embodiments, the T cells of the disclosure may express TCR alpha/beta. In embodiments relating to populations of the T cells of the disclosure, at least 5, 10, 20, 30, 40, 50, 60, 70, 80, 90, 95, 96, 97, 98, or 99% of the T cells may express TCR alpha/beta.

### CD25

CD25 (cluster of differentiation 25; also known as interleukin-2 receptor alpha chain) is a transmembrane protein present on activated T cells and B cells, and a marker of cell activation.

In some embodiments, the NKT cells of the disclosure may be CD25+/−. In embodiments relating to populations of the NKT cells of the disclosure, at least 5, 10, 20, 30, 40, 50, 60, 70, 80, 90, 95, 96, 97, 98, or 99% of the NKT cells may be CD25+/−.

### CD44

CD44 (cluster of differentiation 44) is a cell-surface glycoprotein involved in cell-cell interactions, cell adhesion and migration. It is a receptor for hyaluronic acid and is involved in lymphocyte activation, lymphocyte homing, and recirculation. CD44 expression is an indicative marker for effector-memory T-cells—a subset of infection—and cancer-fighting T cells. Memory T cells have become “experienced” by having encountered antigen during a prior infection, encounter with cancer, or previous vaccination.

In some embodiments, the NKT cells of the disclosure may be CD44+/−. In embodiments relating to populations of the NKT cells of the disclosure, at least 5, 10, 20, 30, 40, 50, 60, 70, 80, 90, 95, 96, 97, 98, or 99% of the NKT cells may be CD44+/−.

### CD69

CD69 (cluster of differentiation 69) is a human transmembrane C-type lectin protein, and an early marker of cell activation. It is expressed in hematopoietic stem cells, T cells, and many other immune cell types. CD69 can induce NKT proliferation and also activate other cells like NK cells and lymphocytes.

In some embodiments, the NKT cells of the disclosure may be CD69+/−. In embodiments relating to populations of the NKT cells of the disclosure, at least 5, 10, 20, 30, 40, 50, 60, 70, 80, 90, 95, 96, 97, 98, or 99% of the NKT cells may be CD69+/−.

In some embodiments, the NKT cells of the disclosure may express CD3, CD4, CD8, CD45, CD49b, CD56, CD62L, NK1.1, Ly6G, Sca1, and/or TCR gamma/delta. In some embodiments, the NKT cells of the disclosure may express CD3, CD4, CD8, CD45, CD56, CD62L, NK1.1, Ly6G, Sca1, and/or TCR gamma/delta. In some embodiments, the NKT cells of the disclosure may express CD3, CD4, CD8, CD45, CD49b, CD62L, NK1.1, Ly6G, Sca1, and/or TCR gamma/delta. In some embodiments, the NKT cells of the disclosure may express CD3, CD4, CD8, CD45, CD56, CD62L, NK1.1, Ly6G, and/or TCR gamma/delta. In some embodiments, the NKT cells of the disclosure may express CD3, CD4, CD8, CD45, CD49b, CD62L, NK1.1, Ly6G, and/or TCR gamma/delta. In some preferred embodiments, the NKT cells of the disclosure express CD4 and CD8. In some preferred embodiments, the NKT cells of the disclosure express CD3, CD4, CD8, and CD49b. In some preferred embodiments, the NKT cells of the disclosure express CD3, CD4, CD8, and CD56. In some preferred embodiments, the NKT cells of the disclosure express Ly6G and TCR gamma/delta. In some preferred embodiments the NKT cells of the disclosure express CD3, CD49b, Ly6G, and TCR gamma/delta. In some preferred embodiments the NKT cells of the disclosure express CD3, CD56, Ly6G, and TCR gamma/delta. In some preferred embodiments the NKT cells of the disclosure express CD3, CD4, CD8, CD49b, Ly6G, and TCR gamma/delta. In some preferred embodiments the NKT cells of the disclosure express CD3, CD4, CD8, CD56, Ly6G, and TCR gamma/delta. In some preferred embodiments the NKT cells of the disclosure express CD3, CD4, CD8, CD45, CD49b, CD62L, NK1.1, Ly6G, Sca1, and TCR gamma/delta. In some preferred embodiments the NKT cells of the disclosure express CD3, CD4, CD8, CD45, CD56, CD62L, NK1.1, Ly6G, Sca1, and TCR gamma/delta. In some preferred embodiments the NKT cells of the disclosure express CD3, CD4, CD8, CD45, CD49b, CD62L, NK1.1, Ly6G, and TCR gamma/delta. In some preferred embodiments the NKT cells of the disclosure express CD3, CD4, CD8, CD45, CD56, CD62L, NK1.1, Ly6G, and TCR gamma/delta.

In some particularly preferred embodiments the NKT cells of the disclosure express CD3, CD45, and/or CD56. In some such embodiments, the NKT cells of the disclosure are CD3+/bright or CD3+/very bright, and/or CD45+/dim, and/or CD56+.

In some embodiments, the NKT cells of the disclosure may not express C-kit, B220, FoxP3, and/or TCR alpha/beta. In some embodiments, the NKT cells of the disclosure do not express C-kit, B220, FoxP3, or TCR alpha/beta. In some preferred embodiments, the NKT cells of the disclosure express CD4 and CD8 and do not express C-kit, B220, FoxP3, and/or TCR alpha/beta. In some preferred embodiments, the NKT cells of the disclosure express Ly6G and TCR gamma/delta and do not express C-kit, B220, FoxP3, and/or TCR alpha/beta.

In some embodiments, the NKT cells of the disclosure are CD44+/−, CD69+/−, and/or CD25+/−. In some embodiments, the NKT cells of the disclosure are CD44+/−, CD69+/−, and CD25+/−.

In embodiments relating to populations of the NKT cells of the disclosure, the population of NKT cells may be characterized in that at least 60, 70, 80, 90, 95, 96, 97, 98, or 99% of the NKT cells express CD3, CD4, CD8, CD45, CD49b, CD56, CD62L, NK1.1, Ly6G, Sca1, and/or TCR gamma/delta. In some such embodiments, the population of NKT cells may be characterized in that at least 60, 70, 80, 90, 95, 96, 97, 98, or 99% of the NKT cells do not express: C-kit, B220, FoxP3, and/or TCR alpha/beta. In some embodiments, the population of NKT cells may be characterized in that at least 60, 70, 80, 90, 95, 96, 97, 98, or 99% of the NKT cells express CD3, CD4, CD8, CD45, CD49b, CD62L, NK1.1, Ly6G, Sca1, and/or TCR gamma/delta. In some embodiments, the population of NKT cells may be characterized in that at least 60, 70, 80, 90, 95, 96, 97, 98, or 99% of the NKT cells express CD3, CD4, CD8, CD45, CD56, CD62L, NK1.1, Ly6G, Sca1, and/or TCR gamma/delta. In some embodiments, the population of NKT cells may be characterized in that at least 60, 70, 80, 90, 95, 96, 97, 98, or 99% of the NKT cells express CD3, CD4, CD8, CD45, CD49b, CD62L, NK1.1, Ly6G, and/or TCR gamma/delta. In some embodiments, the population of NKT cells may be characterized in that at least 60, 70, 80, 90, 95, 96, 97, 98, or 99% of the NKT cells express CD3, CD4, CD8, CD45, CD56, CD62L, NK1.1, Ly6G, and/or TCR gamma/delta.

In some preferred embodiments, the population of NKT cells may be characterized in that at least 60, 70, 80, 90, 95, 96, 97, 98, or 99% of the NKT cells express CD3, CD4, CD8, CD45, CD49b, CD56 CD62L, NK1.1, Ly6G, Sca1, and TCR gamma/delta. In some preferred embodiments, the population of NKT cells may be characterized in that at least 60, 70, 80, 90, 95, 96, 97, 98, or 99% of the NKT cells express CD3, CD4, CD8, CD45, CD49b, CD62L, NK1.1, Ly6G, Sca1, and TCR gamma/delta. In some preferred embodiments, the population of NKT cells may be characterized in that at least 60, 70, 80, 90, 95, 96, 97, 98, or 99% of the NKT cells express CD3, CD4, CD8, CD45, CD56 CD62L, NK1.1, Ly6G, Sca1, and TCR gamma/delta. In some preferred embodiments, the population of NKT cells may be characterized in that at least 60, 70, 80, 90, 95, 96, 97, 98, or 99% of the NKT cells express CD3, CD4, CD8, CD45, CD49b, CD62L, NK1.1, Ly6G, and TCR gamma/delta. In some preferred embodiments, the population of NKT cells may be characterized in that at least 60, 70, 80, 90, 95, 96, 97, 98, or 99% of the NKT cells express CD3, CD4, CD8, CD45, CD56 CD62L, NK1.1, Ly6G, and TCR gamma/delta. In some such embodiments, the population of NKT cells may be characterized in that at least 60, 70, 80, 90, 95, 96, 97, 98, or 99% of the NKT cells do not express: C-kit, B220, FoxP3, or TCR alpha/beta. In some preferred embodiments, the population of NKT cells may be characterized in that at least 60, 70, 80, 90, 95, 96, 97, 98, or 99% of the NKT cells express CD3, CD4, CD8, CD45, CD49b, CD56, CD62L, NK1.1, Ly6G, Sca1, and TCR gamma/delta, and do not express: C-kit, B220, FoxP3, or TCR alpha/beta. In some preferred embodiments, the population of NKT cells may be characterized in that at least 60, 70, 80, 90, 95, 96, 97, 98, or 99% of the NKT cells express CD3, CD4, CD8, CD45, CD49b, CD62L, NK1.1, Ly6G, Sca1, and TCR gamma/delta, and do not express: C-kit, B220, FoxP3, or TCR alpha/beta. In some preferred embodiments, the population of NKT cells may be characterized in that at least 60, 70, 80, 90, 95, 96, 97, 98, or 99% of the NKT cells express CD3, CD4, CD8, CD45, CD56, CD62L, NK1.1, Ly6G, Sca1, and TCR gamma/delta, and do not express: C-kit, B220, FoxP3, or TCR alpha/beta. In some preferred embodiments, the population of NKT cells may be characterized in that at least 60, 70, 80, 90, 95, 96, 97, 98, or 99% of the NKT cells express CD3, CD4, CD8, CD45, CD49b, CD62L, NK1.1, Ly6G, and TCR gamma/delta, and do not express: C-kit, B220, FoxP3, or TCR alpha/beta. In some preferred embodiments, the population of NKT cells may be characterized in that at least 60, 70, 80, 90, 95, 96, 97, 98, or 99% of the NKT cells express CD3, CD4, CD8, CD45, CD56, CD62L, NK1.1, Ly6G, and TCR gamma/delta, and do not express: C-kit, B220, FoxP3, or TCR alpha/beta.

In some particularly preferred embodiments, the population of NKT cells may be characterized in that at least 60, 70, 80, 90, 95, 96, 97, 98, or 99% of the NKT cells express CD3, CD45, and/or CD56. In some such embodiments, at least 60, 70, 80, 90, 95, 96, 97, 98, or 99% of the NKT cells are CD3+/bright or CD3+/very bright, and/or CD45+/dim, and/or CD56+.

In some embodiments, the T cells of the disclosure may express CD3, CD4, CD45, and/or CD49b (CD56 in humans). In some embodiments, the T cells of the disclosure may express CD3, CD4, CD45, CD49b (CD56 in humans), and/or TCR gamma/delta. In some embodiments, the T cells of the disclosure may express CD3, CD4, CD45, CD49b (CD56 in humans), and/or TCR alpha/beta. In some embodiments, the T cells of the disclosure may express CD3, CD4, CD45, CD49b (CD56 in humans), and TCR gamma/delta. In some embodiments, the T cells of the disclosure may express CD3, CD4, CD45, CD49b (CD56 in humans), and TCR alpha/beta. In some embodiments, the T cells of the disclosure may not express CD8.

In embodiments relating to populations of the T cells of the disclosure, the population of T cells may be characterized in that at least 60, 70, 80, 90, 95, 96, 97, 98, or 99% of the T cells express CD3, CD4, CD45, and/or CD49b (CD56 in humans). In some embodiments, the population of T cells may be characterized in that at least 60, 70, 80, 90, 95, 96, 97, 98, or 99% of the T cells express CD3, CD4, CD45, CD49b (CD56 in humans), and/or TCR gamma/delta. In some embodiments, the population of T cells may be characterized in that at least 60, 70, 80, 90, 95, 96, 97, 98, or 99% of the T cells express CD3, CD4, CD45, CD49b (CD56 in humans), and/or TCR alpha/beta. In some such embodiments, the population of T cells may be characterized in that at least 60, 70, 80, 90, 95, 96, 97, 98, or 99% of the T cells do not express CD8.

In some embodiments, the pattern of expression of surface proteins may be as determined by flow cytometry at 24 hours, 48 hours, 72 hours, 96 hours, or 120 hours after administering the glucocorticoid-receptor (GR) modulating agent to the subject. In some embodiments, the pattern of expression of surface proteins may be as determined by flow cytometry performed using the equipment, reagents, and/or conditions described herein (taken in isolation or in combination).

### Gamma Delta T Cells

Gamma delta T cell surface marker characteristics may include (but are not limited to) CD3, CD4, CD8, CD69, CD56, CD27, CD40, CD40L, CD45RA, CD45, CD83, CD16, CD16a, CD16b, ICOS, CD161, Fas, CLEC7A/Dectin-1, FasL, Ecadherin, IL-18R alpha, IL-23R, NKG2D/CD314, NKG2E, Occludin, TKR2, TRAIL, TCR-Vg9, TCR-Vd2, TCR-Vd1, TCR-Vd3, TCR-pan g/d, NKG2D, monoclonal chemokine receptor antibodies CCR5, CCR6, CCR7, CXCR3, CXCR4, or CXCR5 or combinations thereof. The surface marker characteristics of the cells of the invention may include one/more of these. Gamma delta T cells may secrete (including but not limited to) CCL2/JE/MCP-1, CXCL13/BLC/BCA-1, beta-Defensin 2, beta-Defensin 3, alpha-Defensin 1, EGF, KGF/FGF-7, FGF-10, GM-CSF, Granulysin, Granzyme A, Granzyme B, IFN-gamma, IL-2, IL-4, IL-5, IL-6, IL-10, IL-12, IL-12/IL-23 p40, IL-12 p′70, IL-13, IL-17/IL-17A, IL-22, IL-6/IL-6R alpha Complex, LAP (TGF-beta 1), TGF-beta, and/or TNF-alpha. The cells of the invention may secrete one/more of these.

ICAM3 modulating agents in the context of the present disclosure are those which bind ICAM3 and promote the induction and/or mobilisation of the NKT cells, T cells, and dendritic cells of the invention. The ICAM3 modulating agent may be an ICAM3 antagonist/ICAM3 inhibitor, or may be an ICAM3 agonist/activator.

Such ICAM3 modulating agents may include, for example, anti-ICAM3 antibodies raised against ICAM3 or a portion thereof, small molecule modulators of ICAM3 (such as activators or inhibitors of ICAM3), and peptide agents/proteins which bind ICAM3. Suitable means of identifying ICAM3 modulating agents will be well known to those of skill in the art—for example, anti ICAM3 antibodies may be identified by a method which may include bringing into contact a library of antibody molecules and an ICAM3 epitope, and selecting one or more specific antibody molecules of the library able to bind said epitope. Alternatively, these could be identified using competition binding assays employing known anti ICAM3 antibodies, with competition determined, for example, using ELISA or flow cytometry. Similarly, small molecule modulators of ICAM3 may be identified by routine screening experiments such as radioligand binding assays and functional assays.

As already described above, the present authors have discovered the surprising capacity of glucocorticoid receptor modulating agents (such as dexamethasone and other glucocorticoids) to bind ICAM3 and exert modulating actions upon ICAM3. Thus, in some embodiments, the ICAM3 modulating agent may be a glucocorticoid-receptor (GR) modulating agent. In some embodiments, the ICAM3 modulating agent may be a glucocorticoid, for example dexamethasone or betamethasone.

As used herein, the term glucocorticoid-receptor (GR) modulating agent includes glucocorticoids, glucocorticoid receptor agonists, and any compound that binds to the glucocorticoid receptor. Glucocorticoid-receptor (GR) modulating agents such as glucocorticoids exert their effects through both membrane GRs and cytoplasmic GRs which activate or repress gene expression. Some of the desirable lymphodepletive effects of glucocorticoids and GR modulating agents are believed to be mediated via membrane GRs or other non-genomic effects in addition to their genomic effects. Glucocorticoids have been reported to have varied effects on lymphocyte levels, depending on the concentration of the glucocorticoid administered and the duration of treatment. In general, at low doses typically used for chronic therapy, glucocorticoids have been reported to redistribute lymphocytes from the peripheral blood into the bone marrow, at medium doses glucocorticoids have been reported to cause leukocytosis thought to be a redistribution of leukocytes from the bone marrow, spleen and thymus into the peripheral blood, and at high doses glucocorticoids have a lymphotoxic action on lymphocytes by triggering apoptosis and necroptosis. The duration of effect also depends on the dose level; for instance Fauci et al (1976) reports a single oral 0.24 mg/kg dexamethasone dose suppresses peripheral blood T and B lymphocytes 80% with recovery beginning at 12 hours and normal levels by 24 hours. The present authors have previously demonstrated (in international patent application PCT/US2019/054395) that acute oral doses of 3 mg/kg or greater dexamethasone are necessary to reduce peripheral blood T and B cells 24-48 hours after administration, with return to baseline levels occurring around 5 to 14 days after dosing.

Glucocorticoid-receptor (GR) modulating agents which may be used in the disclosed methods include, for example, selective glucocorticoid receptor modulators (SEGRMs) and selective glucocorticoid receptor agonists (SEGRAs). Glucocorticoids, selective glucocorticoid receptor modulators, and selective glucocorticoid receptor agonists (SEGRAs) that may be utilized in the disclosed methods are well known to those skilled in the art.

Some such glucocorticoids include, but are not limited to, dexamethasone, dexamethasone containing agents, hydrocortisone, methylpredisone, prednisone, corticone, budesonide, betamethasone and beclomethasone. Other glucocorticoids include prednisolone, mometasone furoate, Triamcinolone Acetonide, and methylprednisolone.

Accordingly, in some embodiments of the methods of the disclosure, the glucocorticoid-receptor (GR) modulating agent may be a glucocorticoid. In some such embodiments, the glucocorticoid may be selected from the group consisting of: dexamethasone, hydrocortisone, methylprednisolone, prednisone, prednisolone, prednylidene, cortisone, budesonide, betamethasone, flumethasone and beclomethasone. In some preferred embodiments, the glucocorticoid may be selected from the group consisting of: dexamethasone, betamethasone, and methylprednisone. In some particularly preferred embodiments the glucocorticoid may be dexamethasone or betamethasone.

In some embodiments of the methods of the disclosure, the glucocorticoid may be selected from the group consisting of: dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone Isothiocyanatoethyl)Thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone. In some preferred embodiments, the glucocorticoid may be dexamethasone base or dexamethasone sodium phosphate.

In some embodiments of the disclosure, the glucocorticoid receptor modulating agent may not be one or more of the above recited agents.

In the methods of the disclosure, the glucocorticoid-receptor (GR) modulating agent or ICAM3 modulating agent is administered at a dose equivalent to about at least 6 mg/kg human equivalent dose (HED) of dexamethasone base.

Equivalent doses of another glucocorticoid or glucocorticoid receptor modulating agent can be readily and easily calculated using publicly available corticoid conversion algorithms, preferably http://www.medcalc.com. By way of example, 3 to 12 mg/kg dexamethasone converts to 19 to 75 mg/kg prednisone. Since prednisone's biologic half-life is about 20 hours, while dexamethasone's biologic half-life is about 36 to 54 hours prednisone would be dosed between 19 to 75 mg/kg every 24 hours for equivalent biologic dosing. More specifically, a 12 mg/kg dose of dexamethasone corresponds to a 75 mg/kg dose of prednisolone that would require repeat dosing of about two to about three doses every 24 hours. A 10 mg/kg dose of betamethasone is about 12 mg/kg dexamethasone and has a pharmacodynamic (biologic) half-life similar to dexamethasone.

Dexamethasone doses in the examples in the present application are given as human equivalent doses (HED). Methods for calculating the human equivalent dose (HED) are known in the art. For example the FDA's Centre for Drug Evaluation and Research (CDER) issued a highly-cited guidance document in 2005 (U.S Department of Health CDER, 2005), which sets out the established algorithm for converting animal doses to HED based on body surface area (the generally accepted method for extrapolating doses between species) at Table 1 on page 7 of that document. For reference, Table 1 is reproduced below. The skilled person understands that the animal dose in mg/kg, explained below, the HED is calculated easily using the standard conversion factors in the right hand columns of Table 1:

TABLE 1
Conversion of Animal Doses to Human Equivalent
Doses Based on Body Surface Area

| | | To Convert Animal Dose in mg/kg | |
|---|---|---|---|
| | | to HED^{a} in mg/kg, Either: | |
| | To Convert Animal Dose in | Divide | Multiply |
| | mg/kg to Dose in mg/m², | Animal Dose | Animal Dose |
| Species | Multiply by kₘ | By | By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |

Primates:

| | | | |
|---|---|---|---|
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)^{0.33}.
^{b}This kₘ value is provided for reference only since healthy children will rarely be volunteers for phase 1 trials.
^{c}For example, cynomolgus, rhesus, and stumptail.

In some embodiments of the methods of the disclosure, the glucocorticoid-receptor (GR) modulating agent or ICAM3 modulating agent is administered at a dose equivalent to about at least 12 mg/kg human equivalent dose (HED) of dexamethasone base. In other preferred embodiments, the glucocorticoid-receptor (GR) modulating agent is administered at a dose equivalent to about at least 15 mg/kg or about at least 18 mg/kg human equivalent dose (HED) of dexamethasone base. In other preferred embodiments, the glucocorticoid-receptor (GR) modulating agent is administered at a dose equivalent to about at least 21 mg/kg or at least about 24 mg/kg human equivalent dose (HED) of dexamethasone base. In some preferred embodiments, the glucocorticoid-receptor (GR) modulating agent is administered at a dose equivalent to about 12 mg/kg human equivalent dose (HED) of dexamethasone base, about 15 mg/kg human equivalent dose (HED) of dexamethasone base, or about 18 mg/kg human equivalent dose (HED) of dexamethasone base, or about 21 mg/kg human equivalent dose (HED) of dexamethasone base or about 24 mg/kg human equivalent dose (HED) of dexamethasone base, or about 30 mg/kg human equivalent dose (HED) of dexamethasone base, or about 45 mg/kg human equivalent dose (HED) of dexamethasone base.

In some embodiments of the methods of the disclosure, the glucocorticoid-receptor (GR) modulating agent or ICAM3 modulating agent is administered at a dose equivalent to about at least 6-45 mg/kg human equivalent dose (HED) of dexamethasone base; about at least 15-24 mg/kg human equivalent dose (HED) of dexamethasone base; about at least 6-12 mg/kg human equivalent dose (HED) of dexamethasone base; or about at least 12-15 mg/kg human equivalent dose (HED) of dexamethasone base; or about at least 18-30 mg/kg human equivalent dose (HED) of dexamethasone base; or about at least 15-18 mg/kg human equivalent dose (HED) of dexamethasone base. In embodiments in which the infectious disease is a disease resulting from infection with a coronavirus, for example COVID-19, the glucocorticoid-receptor (GR) modulating agent may preferably be administered at a dose equivalent to between about 18-30 mg/kg human equivalent dose (HED) of dexamethasone base.

In the methods of the disclosure, the glucocorticoid-receptor (GR) modulating agent or ICAM3 modulating agent may be administered as a single acute dose, or as a total dose given over about a 24, 48, or 72 hour period. In some preferred embodiments, the glucocorticoid-receptor (GR) modulating agent is administered as a single acute dose. In other preferred embodiments, the glucocorticoid-receptor (GR) modulating agent is administered as a total dose given over about a 72 hour period.

In some embodiments in which the subject has, is suspected of having, or has been diagnosed with an infectious disease, such as a disease resulting from infection with a coronavirus (such as COVID-19), the glucocorticoid receptor modulating agent (which may preferably be dexamethasone or betamethasone) may be administered as a solution in aqueous media. In some such embodiments, the glucocorticoid receptor modulating agent may be provided at a concentration equivalent to about 24 mg/ml dexamethasone phosphate (20 mg/ml dexamethasone base; 26.2 mg/ml dexamethasone sodium phosphate), and administered by intravenous (IV) infusion over a period of about 1 to 2 hours, at an ultimate target dose of between about 18 to 30 mg/kg human equivalent dose (HED) of dexamethasone base. In other embodiments, the glucocorticoid receptor modulating agent may be provided as dexamethasone tablets dissolved in orange juice or citric acid (pH 3.3-4.2) and administered orally or by stomach tube, at an ultimate target dose of between about 18 to 30 mg/kg human equivalent dose (HED) of dexamethasone base.

In some embodiments of the methods of the disclosure, the methods of producing a population of natural killer T cells (NKT cells), producing a population of T cells, and/or producing or activating a population of dendritic cells may comprise a step of administering one or more further doses of a glucocorticoid-receptor (GR) modulating agent or ICAM3 modulating agent to the subject.

In this context, the one or more doses are administered further to a first or preceding dose of glucocorticoid-receptor (GR) modulating agent or ICAM3 modulating agent and may therefore be termed subsequent or second, third, fourth, etc. doses. Accordingly, in some embodiments, the one or more further doses may be administered about 24, 48, 72, 96, 120, 144, or 168 hours after a preceding dose (administration). In some embodiments, the one or more further doses may be administered every about 24, 48, 72, 96, 120, 144, or 168 hours after a preceding dose (administration). In some other embodiments, the one or more further doses may be administered once every week, once every two weeks, once every three weeks, or once every month after a preceding dose (administration). In some other embodiments, the one or more further doses may be administered twice every week after a preceding dose (administration).

In some embodiments, the one or more further doses may be administered between about 24 hours and 168 hours after a preceding dose (administration). In other embodiments, the one or more further doses may be administered between about 24 hours and 120 hours, between about 24 hours and 72 hours, or between about 24 hours and 48 hours after a preceding dose (administration). In some other embodiments, the one or more further doses may be administered between about 48 hours and 168 hours, between about 48 hours and 120 hours, or between about 48 hours and 72 hours after a preceding dose (administration). In some other embodiments, the one or more further doses may be administered between about 72 hours and 168 hours, or between about 72 hours and 120 hours after a preceding dose (administration).

In some embodiments, a subsequent dose is given 7 days after the initial dose. In some embodiments, a subsequent dose is given 14 days after the initial dose. In some embodiments, a subsequent dose is given 21 days after the initial dose.

In some embodiments in which the subject has, is suspected of having, or has been diagnosed with a T cell lymphoma, the one or more further doses may be administered every 21 days, or every 14 days or every 5-7 days for a period of time that can be determined by a physician.

In some embodiments in which the subject has, is suspected of having, or has been diagnosed with a B cell lymphoma, the one or more further doses may be administered every 21 days, or every 14 days or every 5-7 days for a period of time that can be determined by a physician.

In some embodiments of the methods of the disclosure, the method of producing a population of natural killer T cells (NKT cells) may further comprise a step of administering an NKT cell activator to the subject. As used herein, the term NKT cell activator includes any agent or molecule triggering activation of the NKT cells. Activation of NKT cells is associated with upregulation of activation markers and Th1 and Th2 cytokines and chemokines. NKT cell activators that may be utilized in the disclosed methods are well known to those skilled in the art.

Some such NKT cell activators include, but are not limited to, Adipokines, Leptin, adiponectin, apelin, chemerin, MCP-1, PAI-1, RBP4, visfatin, omentin, vaspin, progranulin, CTRP-4, Cytokines, IL-1α, IL-1β, IL-1RA. IL-18, IL-33, IL-36α, IL-36β, IL-36γ. IL-36RA, IL-37, IL-38, IL-2, IL-4, IL-7, IL-9, IL-15, IL-21, IFN-α, IFN-β, IFN-δ, IFN-ε, IFN-κ, IFN-τ, IFN-ω, IFN-γ, IFN-λ1, IFN-λ2, IFN-λ3, IFN-λ4, IL-6, IL-11, IL-31, CLCF1, CNTF, leptin, LIF, OSM, iL-12, IL-17A, IL-17B, IL-17C, IL-17D, IL-17E, IL-17F, 4-1BBL, BAFF, CD40LG, CD70, CD95L/CD178, EDA-A1, LTA/TNF-β, TNF-α, TNFSF4, TNFS8, TNFSF10, TNFSF11, TNFSF12, TNFSF13, TNFSF15, TGF-β1, TGF-β2, TGF-β3, IL-13, G-CSF, GM-CSF, CSF1. Chemokines, CXCL1-CXCL17, CC, CCL1-CCL28, CX3CL1, XCL1, XCL2, Myokines, BDNF, Decorin, irisin, myostatin, myonectin, osteonectin, Prostaglandins, PGI2, PGD2, PGE2, PGF2α, Prostamides, Prostamide 12, prostamide D2, prostamide E2, prostamide F2α, Virokines, Growth Factors, Adrenomedullin, angiopoietin, autocrine motility factor, bone morphogenic proteins, ciliary neurotrophic factor, leukemia inhibitory factor, M-CSF, EGF, ephrine A1-A5, ephrine B1-B3, erythropoietin, FGF1-FGF23, fetal bovine somatotrophin, GDNF, neurturin, persephin, artemin, growth differentiation factor-9, hepatocyte growth factor, hepatocyte-derived growth factor, insulin, insulin-like growth factor ½, keratinocyte growth factor, migration-stimulating factor, macrophage-stimulating protein, neuregulin 1-4, neurotrophin ¾, nerve growth factor, placental growth factor, platelet-derived growth factor, renalase, T-cell growth factor, TGF-α, TGF-β, VEGF, Wnt signaling pathway, anti-NKG2D antibody or its ligand MICA (MEW class I chain-related sequence A), DNAM-1 engagement, 4-1BB engagement, PD-1 inhibitor, NKT Activators, α-galactosylceramide, α-glucoronosylceramide, α-galcturonsylceramide, α-galactosyldiacylgylocerol, phosphatidylinositol-manosidase, α-glucosyldiacylglycerol, cholesterol α-glucoside, β-glaactocsylceramide, isoglobotrihexosylceramide, diasialoganglioside, phosphatidylethanolamine, phosphatidylinositol, phosphatidylcholine, house dust extract, GSL-1, NKp44L, ULBP, Pathogen-derived molecular structures, PAMP, LPS, pathogen-derived RNA, pathogen-derived DNA, viral ligands, Synthetic α-galacosylceramide, KRN7000, PBS44, PBS57, Anti-inflammatory, IL-10, IL-19, IL-20, IL-22, IL-24, IL-28A, IL-28B, IL-29.

In some embodiments of the disclosure, the NKT cell activator may not be one or more of the above recited agents.

Following activation, the NKT cells express NKp46 (NKp44 in humans), lower CD3 and CD49b expression and express IL-10, TGF-β, IFNgamma, IL-4 and several Th1 and Th2 cytokines, Human class-I restricted T cell associated molecule (CRTAM), CCL3/MIP1a, CCL4/MIP1h and CCL5/Rantes and XCL1/lymphotactin, granzyme, CD45RO+CD62L+, CD25, IL2Rbeta, GM-CSF, IL-2, IL-13, TNFalpha, IL-17, IL-21, CD44, CD69, and IL-22. Additionally, in a tumour environment, NKT cells become organized in lines moving in towards tumor cells from all sides.

In some preferred embodiments of the methods of the disclosure, the NKT cell activator may be selected from the group consisting of: alpha GalCer (alpha-Galactosylceramide; α-GalCer) sulfatide β-O-sulfogalactosylceramide; SM4; sulfated galactocerebroside), or an NKT-activating antibody, or may be Perforin, nitric oxide, IL-2, interferons alpha and gamma, TGFbeta, TNFalpha, TNFbeta, G-CSF, VEGF, FGF-18, IL-17, CXCL5, CXCR2, CXCR5, CCR4-CCL17/22, CCR8-CCL1, CCR10-CCL28, and CXCR3-CCL9/10/11, CCL5, CXCR9, CCL2, CCL3, CCL4, CCL5, CXCL9 or CXCL10, interferon (IFN) γinducible chemokines CXCL9, CXCL10, and CXCL11, CCL5 and CXCL9, CCR5, IL-32, IL-6, IL-7, IL-10, IL-18, G-CSF, M-CSF, MCP-1, MCP-3, IP-10, MIG, or MIP-1a. In some other preferred embodiments of the methods of the disclosure, the NKT cell activator may be alpha GalCer loaded dendritic cells or monocytes. In some embodiments of the methods of the disclosure, the NKT cell activator may be administered within 1, 3, 24, 48, 72, 96, 120, 144, or 168 hours of administration of a dose of glucocorticoid-receptor (GR) modulating agent or ICAM3 modulating agent. In some preferred embodiments the NKT cell activator may be administered within or around 1, 3, or 48 hours after administration of a dose of glucocorticoid-receptor (GR) modulating agent or ICAM3 modulating agent. In some particularly preferred embodiments the NKT cell activator may be administered within or around 1, 3, or 48 hours after administration of a dose of glucocorticoid.

In some embodiments of the methods of the disclosure, the method of producing a population of T cells may further comprise a step of administering a T cell activator to the subject. As used herein, the term T cell activator includes any agent or molecule triggering activation of the T cells. T cells can be activated via interaction of TCRs with antigenic peptide and MHC and via non-antigen specific costimulators (such as the cytokine interleukin 1). Activation of T cells is associated with increased cytokine and chemokine production, induction of dendritic cell maturation, recruitment of macrophages, and increased cytolytic activity. Activation of gamma delta T cells may also be associated with increased production of growth factors that maintain epidermal integrity (such as IGF-1, VEGF and FGF-2), as well as antigen presentation for alpha beta T cells. Activation of T cells may also be associated with changes in the pattern of expression of surface markers. For gamma delta T cells, this may include one or more of the following marker phenotypes: CD5−, CD4−/CD8−(double negative), CD3+, CD69, CD56, CD27, CD45RA+, CD45, TCR−Vg9+, TCR-Vd2+, TCR−Vd1+, and/or TCR−Vd3+. T cell activators that may be utilized in the disclosed methods are well known to those skilled in the art.

Some such T cell activators include, but are not limited to, Adipokines, Leptin, adiponectin, apelin, chemerin, MCP-1, PAI-1, RBP4, visfatin, omentin, vaspin, progranulin, CTRP-4, Cytokines, IL-1α, IL-1β, IL-1RA. IL-18, IL-33, IL-36α, IL-36β, IL-36γ. IL-36RA, IL-37, IL-38, IL-2, IL-4, IL-7, IL-9, IL-15, IL-21, IFN-α, IFN-β, IFN-δ, IFN-ε, IFN-κ, IFN-τ, IFN-ω, IFN-γ, IFN-λ1, IFN-λ2, IFN-λ3, IFN-λ4, IL-6, IL-11, IL-31, CLCF1, CNTF, leptin, LIF, OSM, iL-12, IL-17A, IL-17B, IL-17C, IL-17D, IL-17E, IL-17F, 4-1BBL, BAFF, CD40LG, CD70, CD95L/CD178, EDA-A1, LTA/TNF-β, TNF-α, TNFSF4, TNFS8, TNFSF10, TNFSF11, TNFSF12, TNFSF13, TNFSF15, TGF-β1, TGF-β2, TGF-β3, IL-13, G-CSF, GM-CSF, CSF1. Chemokines, CXCL1-CXCL17, CC, CCL1-CCL28, CX3CL1, XCL1, XCL2, Myokines, BDNF, Decorin, irisin, myostatin, myonectin, osteonectin, Prostaglandins, PGI2, PGD2, PGE2, PGF2α, Prostamides, Prostamide 12, prostamide D2, prostamide E2, prostamide F2α, Virokines, Growth Factors, Adrenomedullin, angiopoietin, autocrine motility factor, bone morphogenic proteins, ciliary neurotrophic factor, leukemia inhibitory factor, M-CSF, EGF, ephrine A1-A5, ephrine B1-B3, erythropoietin, FGF1-FGF23, fetal bovine somatotrophin, GDNF, neurturin, persephin, artemin, growth differentiation factor-9, hepatocyte growth factor, hepatocyte-derived growth factor, insulin, insulin-like growth factor ½, keratinocyte growth factor, migration-stimulating factor, macrophage-stimulating protein, neuregulin 1-4, neurotrophin ¾, nerve growth factor, placental growth factor, platelet-derived growth factor, renalase, T-cell growth factor, TGF-α, TGF-β, VEGF, Wnt signaling pathway, NKT Activators, α-galactosylceramide, α-glucoronosylceramide, α-galcturonsylceramide, α-galactosyldiacylgylocerol, phosphatidylinositol-manosidase, α-glucosyldiacylglycerol, cholesterol α-glucoside, β-glaactocsylceramide, isoglobotrihexosylceramide, diasialoganglioside, phosphatidylethanolamine, phosphatidylinositol, phosphatidylcholine, house dust extract, GSL-1, NKp44L, ULBP, Pathogen-derived molecular structures, PAMP, LPS, pathogen-derived RNA, pathogen-derived DNA, viral ligands, Synthetic α-galacosylceramide, KRN7000, PBS44, PBS57, Anti-inflammatory, IL-10, IL-19, IL-20, IL-22, IL-24, IL-28A, IL-28B, IL-29.

In some embodiments of the disclosure, the T cell activator may not be one or more of the above recited agents.

In some embodiments of the methods of the disclosure, the T cell activator may be administered within 1, 3, 24, 48, 72, 96, 120, 144, or 168 hours of administration of a dose of glucocorticoid-receptor (GR) modulating agent or ICAM3 modulating agent. In some preferred embodiments the T cell activator may be administered within or around 1, 3, or 48 hours after administration of a dose of glucocorticoid-receptor (GR) modulating agent or ICAM3 modulating agent. In some particularly preferred embodiments the T cell activator may be administered within or around 1, 3, or 48 hours after administration of a dose of glucocorticoid.

In some embodiments of the methods of the disclosure, the methods of activating a population of dendritic cells may further comprise a step of administering a dendritic cell activator to the subject. As used herein, the term dendritic cell activator includes any agent or molecule triggering activation of the dendritic cells. Dendritic cells can be activated directly by conserved pathogen molecules and indirectly by inflammatory mediators (such as those produced by other cell types that recognize such molecules). Activation of dendritic cells is associated with loss of adhesive structures, reorganization of the cytoskeleton, and increases in cell motility. Activation is also associated with a decrease in endocytic activity but increased expression of MHC-II and co-stimulatory molecules required for T cell activation. Activation of dendritic cells may also be associated with changes in the pattern of expression of surface markers. For CD11b+ dendritic cells, this may include one or more of the following marker phenotypes: CD4−, CD8−, CD11c+, CLEC9a−, CX3CR1+, EpCAM/TROP1−, F4/80+, Fcg RI/CD64+, Integrin aE/CD103−, Integrin aM/CD11b+, Langerin/CD207−, MEW class II+, SIRPa/CD172a+, XCR1. Dendritic cell activators that may be utilized in the disclosed methods are well known to those skilled in the art.

Some such dendritic cell activators include, but are not limited to, Adipokines, Leptin, adiponectin, apelin, chemerin, MCP-1, PAI-1, RBP4, visfatin, omentin, vaspin, progranulin, CTRP-4, Cytokines, IL-1α, IL-1β, IL-1RA. IL-18, IL-33, IL-36a, IL-360, IL-36γ. IL-36RA, IL-37, IL-38, IL-2, IL-4, IL-7, IL-9, IL-15, IL-21, IFN-α, IFN-β, IFN-δ, IFN-ε, IFN-κ, IFN-τ, IFN-ω, IFN-γ, IFN-λ1, IFN-λ2, IFN-λ3, IFN-λ4, IL-6, IL-11, IL-31, CLCF1, CNTF, leptin, LIF, OSM, iL-12, IL-17A, IL-17B, IL-17C, IL-17D, IL-17E, IL-17F, 4-1BBL, BAFF, CD40LG, CD70, CD95L/CD178, EDA-A1, LTA/TNF-β, TNF-α, TNFSF4, TNFS8, TNFSF10, TNFSF11, TNFSF12, TNFSF13, TNFSF15, TGF-01, TGF-02, TGF-03, IL-13, G-CSF, GM-CSF, CSF1. Chemokines, CXCL1-CXCL17, CC, CCL1-CCL28, CX3CL1, XCL1, XCL2, Myokines, BDNF, Decorin, irisin, myostatin, myonectin, osteonectin, Prostaglandins, PGI2, PGD2, PGE2, PGF2a, Prostamides, Prostamide 12, prostamide D2, prostamide E2, prostamide F2a, Virokines, Growth Factors, Adrenomedullin, angiopoietin, autocrine motility factor, bone morphogenic proteins, ciliary neurotrophic factor, leukemia inhibitory factor, M-CSF, EGF, ephrine A1-A5, ephrine B1-B3, erythropoietin, FGF1-FGF23, fetal bovine somatotrophin, GDNF, neurturin, persephin, artemin, growth differentiation factor-9, hepatocyte growth factor, hepatocyte-derived growth factor, insulin, insulin-like growth factor ½, keratinocyte growth factor, migration-stimulating factor, macrophage-stimulating protein, neuregulin 1-4, neurotrophin ¾, nerve growth factor, placental growth factor, platelet-derived growth factor, renalase, T-cell growth factor, TGF-α, TGF-β, VEGF, Wnt signaling pathway, NKT Activators, α-galactosylceramide, α-glucoronosylceramide, α-galcturonsylceramide, α-galactosyldiacylgylocerol, phosphatidylinositol-manosidase, α-glucosyldiacylglycerol, cholesterol α-glucoside, β-glaactocsylceramide, isoglobotrihexosylceramide, diasialoganglioside, phosphatidylethanolamine, phosphatidylinositol, phosphatidylcholine, house dust extract, GSL-1, NKp44L, ULBP, Pathogen-derived molecular structures, PAMP, LPS, pathogen-derived RNA, pathogen-derived DNA, viral ligands, Synthetic α-galacosylceramide, KRN7000, PBS44, PBS57, Anti-inflammatory, IL-10, IL-19, IL-20, IL-22, IL-24, IL-28A, IL-28B, IL-29.

In some embodiments of the disclosure, the dendritic cell activator may not be one or more of the above recited agents.

In some embodiments of the methods of the disclosure, the dendritic cell activator may be administered within 24, 48, 72, 96, 120, 144, or 168 hours of administration of a dose of glucocorticoid-receptor (GR) modulating agent or ICAM3 modulating agent. In some preferred embodiments the dendritic cell activator may be administered within or around 48 hours after administration of a dose of glucocorticoid-receptor (GR) modulating agent or ICAM3 modulating agent. In some particularly preferred embodiments the dendritic cell activator may be administered within or around 48 hours after administration of a dose of glucocorticoid.

The terms “subject” and “patient” are used interchangeably herein, and refer to a human or animal. In some embodiments of the methods of the disclosure, the subject may be mammalian. In some preferred embodiments, the subject may be human of any sex or race. In some embodiments, the human is an adult human. In some embodiments of the methods of the disclosure, the subject may be a healthy subject, such as a healthy adult human subject. In this context a healthy subject is a subject which is not afflicted with disease.

In some embodiments of the methods of the disclosure, the subject may have, be suspected of having, or have been diagnosed with a disease selected from the group consisting of: cancer, autoimmune disease, or infectious disease (also called microbial disease).

As used herein, “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

In some embodiments of the disclosure, the cancer may be: Malignant neoplasm of lip, Malignant neoplasm of tonsil, Malignant neoplasm of tongue, Malignant neoplasm of gum, Malignant neoplasm of mouth, Malignant neoplasm of parotid gland, Malignant neoplasm of salivary glands, Malignant neoplasm of pharynx, Malignant neoplasm of esophagus, Malignant neoplasm of stomach, Malignant neoplasm of small intestine, Malignant neoplasm of colon, Malignant neoplasm of recto sigmoid junction, Malignant neoplasm of rectum, Malignant neoplasm of anus, Malignant neoplasm of liver, Malignant neoplasm of gallbladder, Malignant neoplasm of biliary tract, Malignant neoplasm of pancreas, Malignant neoplasm of intestinal tract, Malignant neoplasm of spleen, Malignant neoplasm of nasal cavity and middle ear, Malignant neoplasm of accessory sinuses, Malignant neoplasm of larynx, Malignant neoplasm of trachea, Malignant neoplasm of bronchus and lung, Malignant neoplasm of thymus, Malignant neoplasm of heart, mediastinum and pleura, Malignant neoplasm of sites in the respiratory system and intrathoracic organs, Malignant neoplasm of bone and articular cartilage of limbs, Malignant neoplasm of bones of skull and face, Malignant neoplasm of vertebral column, Malignant neoplasm of ribs, sternum and clavicle, Malignant neoplasm of pelvic bones, sacrum and coccyx, Malignant melanoma of skin, Malignant melanoma of lip, Malignant melanoma of eyelid, including canthus, Malignant melanoma of ear and external auricular canal, Malignant melanoma of face, Malignant melanoma of anal skin, Malignant melanoma of skin of breast, Malignant melanoma of limbs, including shoulder, Merkel cell carcinoma, Basal cell carcinoma of skin of lip, Squamous cell carcinoma of skin of lip, Other and unspecified malignant neoplasm skin/eyelid, including canthus, Malignant neoplasm skin/ear and external auric canal, Other and unspecified malignant neoplasm skin/and unspecified parts of face, Basal cell carcinoma of skin of other and unspecified parts of face, Squamous cell carcinoma of skin of and unspecified parts of face, Basal cell carcinoma of skin of scalp and neck, Squamous cell carcinoma of skin of scalp and neck, Basal cell carcinoma of skin of trunk, Basal cell carcinoma of anal skin, Basal cell carcinoma of skin of breast, Squamous cell carcinoma of skin of trunk, Squamous cell carcinoma of anal skin, Squamous cell carcinoma of skin of breast, Squamous cell carcinoma of skin of other part of trunk, Other and unspecified malignant neoplasm skin/limbs including shoulder, Basal cell carcinoma skin/limbs, including shoulder, Squamous cell carcinoma skin/limbs, including shoulder, Basal cell carcinoma of skin of limbs, including hip, Squamous cell carcinoma of skin of limbs, including hip, Mesothelioma, Kaposi's sarcoma, Malignant neoplasm of peripheral nerves and autonomic nervous sys, Malignant neoplasm of retroperitoneum and peritoneum, Malignant neoplasm of other connective and soft tissue, Malignant neoplasm of connective and soft tissue of thorax, Malignant neoplasm of connective and soft tissue of abdomen, Malignant neoplasm of connective and soft tissue of pelvis, Malignant neoplasm of conn and soft tissue of trunk, unspecified, Malignant neoplasm of overlapping sites of connective and soft tissue, Malignant neoplasm of connective and soft tissue, unspecified, Gastrointestinal stromal tumor, Malignant neoplasm of breast, Malignant neoplasm of vulva, Malignant neoplasm of vagina, Malignant neoplasm of cervix uteri, Malignant neoplasm of corpus uteri, Malignant neoplasm of uterus, part unspecified, Malignant neoplasm of ovary, Malignant neoplasm of other and unspecified female genital organs, Malignant neoplasm of placenta, Malignant neoplasm of penis, Malignant neoplasm of prostate, Malignant neoplasm of testis, Malignant neoplasm of other and unspecified male genital organs, Malignant neoplasm of kidney, Malignant neoplasm of renal pelvis, Malignant neoplasm of ureter, Malignant neoplasm of bladder, Malignant neoplasm of other and unspecified urinary organs, Malignant neoplasm of eye and adnexa, Malignant neoplasm of meninges, Malignant neoplasm of brain, Malignant neoplm of spinal cord, cranial nerves, Malignant neoplasm of optic nerve, Malignant neoplasm of other and unspecified cranial nerves, Malignant neoplasm of central nervous system, unspecified, Malignant neoplasm of thyroid gland, Malignant neoplasm of adrenal gland, Malignant neoplasm of endo glands and related structures, Malignant neuroendocrine tumors, Malignant carcinoid tumors, Secondary neuroendocrine tumors, Malignant neoplasm of head, face and neck, Malignant neoplasm of thorax, Malignant neoplasm of abdomen, Malignant neoplasm of pelvis, Malignant neoplasm of limbs, Malignant neoplasm of lower limb, Secondary and unspecified malignant neoplasm of lymph nodes, Secondary malignant neoplasm of respiratory and digestive organs, Secondary malignant neoplasm of kidney and renal pelvis, Secondary malignant neoplm of bladder and other and unspecified urinary organs, Secondary malignant neoplasm of skin, Secondary malignant neoplasm of brain and cerebral meninges, Secondary malignant neoplasm of and unspecified parts of nervous sys, Secondary malignant neoplasm of bone and bone marrow, Secondary malignant neoplasm of ovary, Secondary malignant neoplasm of adrenal gland, Hodgkin lymphoma, Follicular lymphoma, Non-follicular lymphoma, Small cell B-cell lymphoma, Mantle cell lymphoma, Diffuse large B-cell lymphoma, Lymphoblastic (diffuse) lymphoma, Burkitt lymphoma, Other non-follicular lymphoma, Non-follicular (diffuse) lymphoma, unspecified, Mature T/NK-cell lymphomas, Sezary disease, Peripheral T-cell lymphoma, not classified, Anaplastic large cell lymphoma, ALK-positive, Anaplastic large cell lymphoma, ALK-negative, Cutaneous T-cell lymphoma, unspecified, Other mature T/NK-cell lymphomas, Mature T/NK-cell lymphomas, unspecified, Other and unspecified types of non-Hodgkin lymphoma, Malignant immunoproliferative dis and certain other B-cell lymph, Multiple myeloma and malignant plasma cell neoplasms, Lymphoid leukemia, Acute lymphoblastic leukemia [ALL], Chronic lymphocytic leukemia of B-cell type, Prolymphocytic leukemia of B-cell type, Hairy cell leukemia, Adult T-cell lymphoma/leukemia (HTLV-1-associated), Prolymphocytic leukemia of T-cell type, Mature B-cell leukemia Burkitt-type, Other lymphoid leukemia, Lymphoid leukemia, unspecified, Myeloid leukemia, Acute myeloblastic leukemia, Chronic myeloid leukemia, BCR/ABL-positive, Atypical chronic myeloid leukemia, BCR/ABL-negative, Myeloid sarcoma, Acute promyelocytic leukemia, Acute myelomonocytic leukemia, Acute myeloid leukemia with 11q23-abnormality, Other myeloid leukemia, Myeloid leukemia, unspecified, Monocytic leukemia, Chronic myelomonocytic leukemia, Juvenile myelomonocytic leukemia, Other monocytic leukemia, Monocytic leukemia, unspecified, Other leukemias of specified cell type, Acute erythroid leukemia, Acute megakaryoblastic leukemia, Mast cell leukemia, Acute panmyelosis with myelofibrosis, Myelodysplastic disease, not classified, Other specified leukemias, Leukemia of unspecified cell type, Chronic leukemia of unspecified cell type, Leukemia, unspecified, Other & unspecified malignant neoplasm of lymphoid, hematopoietic tissue, Carcinoma in situ of oral cavity, esophagus and stomach, Carcinoma in situ of colon, Carcinoma in situ of recto sigmoid junction, Carcinoma in situ of rectum, Carcinoma in situ of anus and anal canal, Carcinoma in situ of other and unspecified parts of intestine, Carcinoma in situ of unspecified part of intestine, Carcinoma in situ of other parts of intestine, Carcinoma in situ of liver, gallbladder and bile ducts, Carcinoma in situ of other specified digestive organs, Carcinoma in situ of digestive organ, unspecified, Carcinoma in situ of middle ear and respiratory system, Carcinoma in situ of larynx, Carcinoma in situ of trachea, Carcinoma in situ of bronchus and lung, Carcinoma in situ of other parts of respiratory system, Melanoma in situ, Melanoma in situ of lip, Melanoma in situ of eyelid, including canthus, Melanoma in situ of ear and external auricular canal, Melanoma in situ of unspecified part of face, Melanoma in situ of scalp and neck, Melanoma in situ of trunk, Melanoma in situ of anal skin, Melanoma in situ of breast (skin) (soft tissue), Melanoma in situ of upper limb, including shoulder, Melanoma in situ of lower limb, including hip, Melanoma in situ of other sites, Carcinoma in situ of skin, Carcinoma in situ of skin of lip, Carcinoma in situ of skin of eyelid, including canthus, Carcinoma in situ skin of ear and external auricular canal, Carcinoma in situ of skin of other and unspecified parts of face, Carcinoma in situ of skin of scalp and neck, Carcinoma in situ of skin of trunk, Carcinoma in situ of skin of upper limb, including shoulder, Carcinoma in situ of skin of lower limb, including hip, Carcinoma in situ of skin of other sites, Carcinoma in situ of breast, Lobular carcinoma in situ of breast, Intraductal carcinoma in situ of breast, Other specified type of carcinoma in situ of breast, Unspecified type of carcinoma in situ of breast, Carcinoma in situ of cervix uteri, Carcinoma in situ of other parts of cervix, Carcinoma in situ of cervix, unspecified, Carcinoma in situ of other and unspecified genital organs, Carcinoma in situ of endometrium, Carcinoma in situ of vulva, Carcinoma in situ of vagina, Carcinoma in situ of other and unspecified female genital organs, Carcinoma in situ of penis, Carcinoma in situ of prostate, Carcinoma in situ of unspecified male genital organs, Carcinoma in situ of scrotum, Carcinoma in situ of other male genital organs, Carcinoma in situ of bladder, Carcinoma in situ of other and unspecified urinary organs, Carcinoma in situ of eye, Carcinoma in situ of thyroid and other endocrine glands, Benign neoplasm of mouth and pharynx, Benign neoplasm of major salivary glands, Benign neoplasm of colon, rectum, anus and anal canal, Benign neoplasm of and ill-defined parts of digestive system, Benign neoplasm of esophagus, Benign neoplasm of stomach, Benign neoplasm of duodenum, Benign neoplasm of other and unspecified parts of small intestine, Benign neoplasm of liver, Benign neoplasm of extrahepatic bile ducts, Benign neoplasm of pancreas, Benign neoplasm of endocrine pancreas, Benign neoplasm of ill-defined sites within the digestive system, Benign neoplasm of middle ear and respiratory system, Benign neoplasm of respiratory system, unspecified, Benign neoplasm of other and unspecified intrathoracic organs, Benign neoplasm of thymus, Benign neoplasm of heart, Benign neoplasm of mediastinum, Benign neoplasm of other specified intrathoracic organs, Benign neoplasm of intrathoracic organ, unspecified, Benign neoplasm of bone and articular cartilage, Benign neoplasm of short bones of upper limb, Benign neoplasm of long bones of lower limb, Benign neoplasm of short bones of lower limb, Benign neoplasm of bones of skull and face, Benign neoplasm of lower jaw bone, Benign neoplasm of vertebral column, Benign neoplasm of ribs, sternum and clavicle, Benign neoplasm of pelvic bones, sacrum and coccyx, Benign neoplasm of bone and articular cartilage, unspecified, Benign lipomatous neoplasm, Ben lipomatous neoplm of skin, subcutaneous of head, face and neck, Benign lipomatous neoplasm of intrathoracic organs, Benign lipomatous neoplasm of intra-abdominal organs, Benign lipomatous neoplasm of spermatic cord, Benign lipomatous neoplasm of other sites, Benign lipomatous neoplasm of kidney, Benign lipomatous neoplasm of other genitourinary organ, Hemangioma and lymphangioma, any site, Hemangioma, Hemangioma unspecified site, Hemangioma of skin and subcutaneous tissue, Hemangioma of intracranial structures, Hemangioma of intra-abdominal structures, Hemangioma of other sites, Lymphangioma, any site, Benign neoplasm of mesothelial tissue, Benign neoplm of soft tissue of retroperitoneum and peritoneum, Other benign neoplasms of connective and other soft tissue, Melanocytic nevi, Melanocytic nevi of lip, Melanocytic nevi of eyelid, including canthus, Melanocytic nevi of unspecified eyelid, including canthus, Melanocytic nevi of ear and external auricular canal, Melanocytic nevi of other and unspecified parts of face, Melanocytic nevi of scalp and neck, Melanocytic nevi of trunk, Melanocytic nevi of upper limb, including shoulder, Melanocytic nevi of lower limb, including hip, Melanocytic nevi, unspecified, Other benign neoplasm of skin of eyelid, including canthus, Other benign neoplasm skin/ear and external auricular canal, Other benign neoplasm skin/left ear and external auric canal, Other benign neoplasm of skin of other and unspecified parts of face, Other benign neoplasm of skin of other parts of face, Other benign neoplasm of skin of scalp and neck, Other benign neoplasm of skin of trunk, Other benign neoplasm skin/upper limb, including shoulder, Other benign neoplasm of skin of lower limb, including hip, Other benign neoplasm of skin, unspecified, Benign neoplasm of breast, Benign neoplasm of unspecified breast, Leiomyoma of uterus, Other benign neoplasms of uterus, Benign neoplasm of ovary, Benign neoplasm of other and unspecified female genital organs, Benign neoplasm of male genital organs, Benign neoplasm of urinary organs, Benign neoplasm of kidney, Benign neoplasm of renal pelvis, Benign neoplasm of ureter, Benign neoplasm of bladder, Benign neoplasm of urethra, Benign neoplasm of other specified urinary organs, Benign neoplasm of urinary organ, unspecified, Benign neoplasm of eye and adnexa, Benign neoplasm of conjunctiva, Benign neoplasm of cornea, Benign neoplasm of retina, Benign neoplasm of choroid, Benign neoplasm of ciliary body, Benign neoplasm of lacrimal gland and duct, Benign neoplasm of unspecified site of orbit, Benign neoplasm of unspecified part of eye, Benign neoplasm of meninges, Benign neoplasm of brain and central nervous system, Benign neoplasm of thyroid gland, Benign neoplasm of other and unspecified endocrine glands, Benign neoplasm of other and unspecified sites, Benign neoplasm of lymph nodes, Benign neoplasm of peripheral nerves and autonomic nervous sys, Benign neoplasm of other specified sites, Benign neuroendocrine tumors, Other benign neuroendocrine tumors, Neoplasm of uncertain behavior of oral cavity and digestive organs, Neoplasm of uncertain behavior of the major salivary glands, Neoplasm of uncertain behavior of pharynx, Neoplasm of uncertain behavior of sites of the oral cavity, Neoplasm of uncertain behavior of stomach, Neoplasm of uncertain behavior of small intestine, Neoplasm of uncertain behavior of appendix, Neoplasm of uncertain behavior of colon, Neoplasm of uncertain behavior of rectum, Neoplasm of uncertain behavior of liver, GB & bile duct, Neoplasm of uncertain behavior of other digestive organs, Neoplasm of uncertain behavior of digestive organ, Neoplm of mid ear and intrathoracic organs, Neoplasm of uncertain behavior of larynx, Neoplasm of uncertain behavior of trachea, bronchus and lung, Neoplasm of uncertain behavior of pleura, Neoplasm of uncertain behavior of mediastinum, Neoplasm of uncertain behavior of thymus, Neoplasm of uncertain behavior of other respiratory organs, Neoplasm of uncertain behavior of respiratory organ, unspecified, Neoplasm of uncertain behavior of female genital organs, Neoplasm of uncertain behavior of uterus, Neoplasm of uncertain behavior of ovary, Neoplasm of uncertain behavior of unspecified ovary, Neoplasm of uncertain behavior of placenta, Neoplasm of uncertain behavior of male genital organs, Neoplasm of uncertain behavior of urinary organs, Neoplasm of uncertain behavior of kidney, Neoplasm of uncertain behavior of unspecified kidney, Neoplasm of uncertain behavior of renal pelvis, Neoplasm of uncertain behavior of ureter, Neoplasm of uncertain behavior of bladder, Neoplasm of uncertain behavior of other urinary organs, Neoplasm of uncertain behavior of unspecified urinary organ, Neoplasm of uncertain behavior of meninges, Neoplasm of uncertain behavior of cerebral meninges, Neoplasm of uncertain behavior of spinal meninges, Neoplasm of uncertain behavior of meninges, unspecified, Neoplasm of uncertain behavior of brain, Neoplasm of uncertain behavior of brain, Neoplasm of uncertain behavior of brain, infratentorial, Neoplasm of uncertain behavior of brain, unspecified, Neoplasm of uncertain behavior of cranial nerves, Neoplasm of uncertain behavior of spinal cord, Neoplasm of uncertain behavior of central nervous system, Neoplasm of uncertain behavior of endocrine glands, Neoplasm of uncertain behavior of thyroid gland, Neoplasm of uncertain behavior of adrenal gland, Neoplasm of uncertain behavior of unspecified adrenal gland, Neoplasm of uncertain behavior of parathyroid gland, Neoplasm of uncertain behavior of pituitary gland, Neoplasm of uncertain behavior of craniopharyngeal duct, Neoplasm of uncertain behavior of pineal gland, Neoplasm of uncertain behavior of carotid body, Neoplasm of uncertain behavior of aortic body and other paraganglia, Neoplasm of uncertain behavior of unspecified endocrine gland, Polycythemia vera, Myelodysplastic syndromes, Refractory anemia without ring sideroblasts, so stated, Refractory anemia with ring sideroblasts, Refractory anemia with excess of blasts [RAEB], Myelodysplastic syndrome, unspecified, Other neoplm of uncertain behavior of lymphoid, hematopoietic tissue, Histiocytic and mast cell tumors of uncertain behavior, Chronic myeloproliferative disease, Monoclonal gammopathy, Essential (hemorrhagic) thrombocythemia, Osteomyelofibrosis, Other neoplasm of uncertain behavior of lymphoid, hematopoietic tissue, Neoplasm of uncertain behavior of lymphoid, hematopoietic & unspecified, Neoplasm of uncertain behavior of other and unspecified sites, Neoplasm of uncertain behavior of bone/artic cartilage, Neoplasm of uncertain behavior of connective/soft tissue, Neoplasm of uncertain behavior of peripheral nerves and autonomous nervous sys, Neoplasm of uncertain behavior of retroperitoneum, Neoplasm of uncertain behavior of peritoneum, Neoplasm of uncertain behavior of skin, Neoplasm of uncertain behavior of breast, Neoplasm of unspecified behavior of digestive system, Neoplasm of unspecified behavior of respiratory system, Neoplasm of unspecified behavior of bone, soft tissue, and skin, Neoplasm of unspecified behavior of breast, Neoplasm of unspecified behavior of bladder, Neoplasm of unspecified behavior of other genitourinary organs, Neoplasm of unspecified behavior of kidney, Neoplasm of unspecified behavior of other GU organ, Neoplasm of unspecified behavior of brain, Neoplasm of unspecified behavior of endo glands and other parts of nervous sys, Neoplasm of unspecified behavior of retina and choroid, or Neoplasm of unspecified behavior of unspecified site.

In some embodiments of the disclosure, the cancer may not be one of the above recited cancers.

In some preferred embodiments of the disclosure, the cancer may be selected from the group consisting of: lymphoma, squamous cell cancer (such as epithelial squamous cell cancer); lung cancer, including small-cell lung cancer, non-small cell lung cancer, adenocarcinoma of the lung and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; and head and neck cancer. In some particularly preferred embodiments of the disclosure the cancer may be lymphoma. In more particularly preferred embodiments of the disclosure the cancer may be a B cell lymphoma or a T cell lymphoma. In some particularly preferred embodiments of the disclosure the cancer may be non-Hodgkin lymphoma. In other preferred embodiments, the cancer may be a post-transplant lymphoproliferative disorder. In some other particularly preferred embodiments of the disclosure the cancer may be a solid tumor cancer.

In embodiments in which the methods of the disclosure are carried out on a subject having, suspected of having, or having been diagnosed with cancer, the NKT cells, T cells, and/or dendritic cells produced by these methods may treat the cancer. In this context, “treat” means to exert a beneficial therapeutic effect in the subject, which can be any overall clinical benefit derived from the methods of the disclosure. This overall clinical benefit can be any of, for example: prolonged survival, partial or complete disease remission, (for example, as assessed by % bone marrow myeloblasts and/or normal maturation of cell lines), slowing or absence of disease progression (for example, as assessed by change in % bone marrow myeloblasts), tumour shrinkage (for example, a reduction in tumour volume of 5, 10, 20, 30, 40% or more), reduction in tumour burden (for example, a reduction in tumour burden of 5, 10, 20, 30, 40% or more), slowing or absence of tumour enlargement, slowing or absence of increase in tumour burden, improved quality of life (for example, as assessed using a health-related quality of life questionnaire such as a Functional Assessment of Cancer Therapy (FACT) questionnaire), progression-free survival, overall survival, hematologic improvement (for example: increased blood haemoglobin, platelet count, and/or neutrophil count), bone marrow response (for example: bone marrow with ≤5% myeloblasts; 30%, 40%, 50% or more reduction in bone marrow myeloblasts; absence of circulating myeloblasts and myeloblasts with Auer rods; absence of extramedullary disease), hematologic recovery (for example: ≥11 g/dL haemoglobin, ≥100×109/L platelets, and/or ≥1×109/L neutrophils in peripheral blood), negative response for a genetic marker (for example, CEBPA, NPM1, or FLT3), or any other positive patient outcome.

The overall clinical benefit may be an “anti-tumor effect”. As used herein, an “anti-tumor effect” refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine. Suitable methods for determining tumour volume/burden are well known to the skilled person, for example, using: computed tomography (CT), or magnetic resonance imaging (MM) imaging technologies; X-ray imaging, for example, mammography; ultrasound imaging; nuclear imaging, for example positron emission tomography (PET), PET/CT scans, bone scans, gallium scans, or metaiodobenzylguanidine (MIBG) scans; bioluminescence imaging (BLI); fluorescence imaging (FLI); BD ToF (infrared-based 3D Time-of-Flight camera) imaging.

Accordingly, in some embodiments, the NKT cells of the disclosure may treat the cancer via tumour infiltration. In some embodiments, the NKT cells of the disclosure may treat the cancer via release of immune activating cytokines. In some embodiments, the NKT cells of the disclosure may engulf and kill cancer cells in the subject. In some embodiments, the NKT cells of the disclosure promote infiltration of other immune cells into a tumor. In some embodiments, the NKT cells of the disclosure directly kill cancer cells via CD1d-directed apoptosis.

In some embodiments, the T cells of the disclosure may treat the cancer via tumour infiltration. In some embodiments, the T cells of the disclosure may treat the cancer via release of immune activating cytokines. In some embodiments, the T cells of the disclosure promote infiltration of other immune cells into a tumor. In some embodiments, the T cells of the disclosure directly kill cancer cells by inducing apoptosis, for example by expressing ligands which engage death receptors on target cells. In some embodiments, the T cells of the disclosure may ingest or engulf cancer cells in the subject. In some embodiments, the T cells may secrete cytotoxic molecules which kill the cancer cells.

In some embodiments, the dendritic cells of the disclosure may treat the cancer via immune surveillance. Dendritic cells (DCs) are antigen-presenting cells derived from bone marrow precursors and form a widely distributed cellular system throughout the body. DCs exert immune-surveillance for exogenous and endogenous antigens and the later activation of naive T lymphocytes giving rise to various immunological responses. DCs are sentinel cells responsible for the recognition of pathogens and signals of tissue damage, which induces their migration to lymphoid organs to carry out the activation of different subsets of T, natural killer (NK), NKT, and B lymphocytes. Mature phenotype cDC are characterized by an increase in MHCII, CD80, CD86, and CD40. In some embodiments, the dendritic cells of the disclosure promote infiltration of other immune cells, such as T cells, into a tumor. In some embodiments the dendritic cells of the disclosure enhance the T cell response to cancer by presenting cancer antigens to T cells. In some embodiments the dendritic cells of the disclosure may directly kill cancer cells by inducing apoptosis, for example by expressing ligands which engage death receptors on target cells

“Autoimmune disease” as used herein refers to autoimmune disorders and other diseases arising from an abnormal immune in which the immune system aberrantly attacks a subject's own constituents. (In healthy subjects, the immune system avoids damaging autoimmune reactions by establishing tolerance to the subject's own constituents). Examples of various autoimmune diseases are described herein and include but are not limited to, celiac disease, diabetes mellitus type 1, Graves' disease, inflammatory bowel disease, transient osteoporosis, multiple sclerosis, psoriasis, rheumatoid arthritis, and systemic lupus erythematosus.

Autoreactive immune cells express high levels of phosphoantigens, which are diphosphate-containing metabolites, as do stressed cells and microorganisms like Mycobacteria, E. coli, and Plasmodium, in particular the phosphoantigen produced by (E)-4-hydroxy-3-methyl-but-2-enyl pyrophosphate (HMB-PP). Humans do not produce HMB-PP. but the majority of gram-negative bacteria do produce it including Mycobacterium tuberculosis, Mycobacterium bovus, Clostidrium difficile, Listeria monocytogenes, malaria parasites and Toxoplasma gondii and Schistosoma japonicum. Gamma delta T cells/receptors are very responsive to HMB-PP, zoledronate and isopentyl pyrophosphate (IPP), mycolylarabinogalactan peptidoglycan (mAGP), and iso-butylamine (IBA). Butyrophilin family members like BTN2A1, BTN3A1, BTNL3, BTNL8, BTNL1, BTNL6, Skint1, Skint2, play an important role in gamma delta T cell recognition of phosphoantigens. Aminobisphosphonate stimulation of peripheral blood mononuclear cells (PBMC) can also activate gamma delta T cell receptors. IL-18 can enhance the response of the gamma delta T cell receptor to phosphoantigens.

In some embodiments of the disclosure the autoimmune disease may be: allergies, asthma, graft versus host disease (GvHD), steroid-resistant GvHD, Achalasia, Addison's disease, Adult Still's disease, Agammaglobulinemia, Alopecia areata, Alopecia, transient osteoporosis, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optic), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, Hemophagocytic lymphohistiocytosis, multiple myeloma, allergen specific immunotherapy, autosomal dominant haploinsufficiency, anterior interosseous nerve syndrome, Churg-Strauss syndrome, Systemic vasculitis, chronic graft versus host disease, Opsoclonus-Myoclonus Syndrome, Necrotising Autoimmune Myopathy (NAM), Pulmonary Sarcomatoid carcinomas, Waldenstrom's macroglobulinaemia (WM), fertility, Behcets Disease, Alopecia areata (AA), Acute-on-chronic Liver Failure, melanoma, ‘organizing bronchiolitis syndrome’, or encephalitis. In some embodiments the autoimmune disease may be: rheumatoid arthritis, rheumatic fever, multiple Sclerosis, experimental autoimmune encephalomyelitis, psoriasis, uveitis, diabetes mellitus, Systemic lupus erythematosus (SLE), lupus nephritis, eczema, Scleroderma, polymyositis/scleroderma, polymyositis/dermatomyositis, uncerative protitis, severe combined immunodeficiency (SCID), DiGeorge syndrome, ataxia-telangiectasia, seasonal allergies, perennial allergies, food allergies, anaphylaxis, mastocytosis, allergic rhinitis, atopic dermatitis, Parkinson's, Alzheimer's, hypersplenism, leukocyte adhesion deficiency, X-linked lymphoproliferative disease, X-linked agammaglobulinemia, selective immuno globulin A deficiency, hyper IgM syndrome, HIV, autoimmune lymphoproliferative syndrome, Wiskott-Aldrich syndrome, chronic granulomatous disease, common variable immunodeficiency (CVID), hyperimmunoglobulin E syndrome, Hashimoto's thyroiditis, acute idiopathic thrombocytopenic purpura, chronic idiopathic thrombocytopenia pur pura, dermatomyositis, Sydenham's chorea, myasthenia gravis, polyglandular syndromes, bullous pemphigoid, Henoch-Schonlein purpura, poststreptococcalnephritis, erythema nodosum, erythema multiforme, gA nephropathy, Takayasu's arteritis, Addison's disease, sarcoidosis, ulcerative colitis, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, chronic active hepatitis, polychondritis, pamphigus Vulgaris, Wegener's granulomatosis, membranous nephropathy, amyotrophic lateral Sclerosis, tabesdorsalis, giant cell arteritis, polymyalgia, peraiciousa nemia, rapidly progressive glomerulonephritis, psoriasis, fibrosing alveolitis, or cancer.

In some embodiments of the disclosure, the autoimmune disease may not be one of the above recited autoimmune diseases.

In some preferred embodiments of the disclosure, the autoimmune disease may be selected from the group consisting of: multiple sclerosis, systemic sclerosis, amyotrophic lateral sclerosis, type 1 diabetes mellitus (T1D), scleroderma, pemphigus, and lupus. In some other preferred embodiments of the disclosure the autoimmune disease may be selected from the group consisting of: graft versus host disease (GvHD), and an allergic disorder such as asthma. In some particularly preferred embodiments of the disclosure the autoimmune disease may be type 1 diabetes mellitus (T1D).

In embodiments in which the methods of the disclosure are carried out on a subject having, suspected of having, or having been diagnosed with autoimmune disease, the NKT cells, T cells, and/or dendritic cells produced by these methods may treat the autoimmune disease. In this context, “treat” means to exert a beneficial therapeutic effect in the subject, which can be any overall clinical benefit derived from the methods of the disclosure. This overall clinical benefit can be any of, for example: reduced fatigue, reduced achy muscles, reduced swelling and redness, reduced low-grade fever, reduced trouble concentrating, reduced numbness and tingling in the hands and feet and arms or legs, reduced urination, reduced hair loss, reduced skin rashes, restored normoglycemia, increased C peptide, improved wound healing, reduced diarrhea, reduced muscle spasms, improved muscle tone and control, reduced skin rash or scaly plaques on the skin or discoloration, improved weight maintenance, reduced muscle or joint pain, improved comfort of the digestive tract, normal heart rate, reduced anxiety, reduced expanded disability status scale (EDSS) score, reduced unique active lesions in the brain measured by gadolinium enhanced MM.

In some embodiments, the NKT cells of the disclosure may treat the autoimmune disease via direct killing of autoreactive T and/or B lymphocytes, increasing Treg: T lymphocyte ratio, inhibiting the activity of autoreactive T and/or B lymphocytes, reducing inflammation, or reducing the trafficking of autoreactive lymphocytes.

In some embodiments, the T cells of the disclosure may treat the autoimmune disease via direct killing of autoreactive T and/or B lymphocytes, increasing Treg: T lymphocyte ratio, inhibiting the activity of autoreactive T and/or B lymphocytes, reducing inflammation, or reducing the trafficking of autoreactive lymphocytes.

In some embodiments, the dendritic cells of the disclosure may treat the autoimmune disease via release of immune activating cytokines, or by promoting T cell killing of autoreactive T and/or B lymphocytes.

“Infectious disease” (or “microbial disease”) as used herein refers to a disease or illness resulting from the infection of a subject's body by infectious agents (pathogens) such as viruses, bacteria, or fungi. In some embodiments of the disclosure the infectious disease may be: Acinetobacter infections (Acinetobacter baumannii), Actinomycosis (Actinomyces israelii, Actinomyces gerencseriae and Propionibacterium propionicus) African sleeping sickness or African trypanosomiasis (Trypanosoma brucei), AIDS (Acquired immunodeficiency syndrome) (Human immunodeficiency virus), Amebiasis (Entamoeba histolytica), Anaplasmosis (Anaplasma species), Angiostrongyliasis (Angiostrongylus), Anisakiasis (Anisakis), Anthrax (Bacillus anthracis), Arcanobacterium haemolyticum infection (Arcanobacterium haemolyticum), Argentine hemorrhagic fever (Junin virus), Ascariasis (Ascaris lumbricoides), Aspergillosis (Aspergillus species), Astrovirus infection (Astroviridae family), Babesiosis (Babesia species), Bacillus cereus infection (Bacillus cereus), Bacterial pneumonia (multiple bacteria), Bacterial vaginosis (List of bacterial vaginosis microbiota), Bacteroides infection (Bacteroides species), Balantidiasis (Balantidium coli), Bartonellosis (Bartonella), Baylisascaris infection (Baylisascaris species), BK virus infection (BK virus), Black piedra (Piedraia hortae), Blastocystosis (Blastocystis species), Blastomycosis (Blastomyces dermatitidis), Bolivian hemorrhagic fever (Machupo virus), Botulism (and Infant botulism) (Clostridium botulinum; Note: Botulism is not an infection by Clostridium botulinum but caused by the intake of botulinum toxin), Brazilian hemorrhagic fever (Sabia virus), Brucellosis (Brucella species), Bubonic plague (the bacterial family Enterobacteriaceae), Burkholderia infection, usually Burkholderia cepacia and other Burkholderia species, Buruli ulcer (Mycobacterium ulcerans), Calicivirus infection (Norovirus and Sapovirus) (Caliciviridae family), Campylobacteriosis (Campylobacter species), Candidiasis (Moniliasis; Thrush) (usually Candida albicans and other Candida species), Capillariasis (Intestinal disease by Capillaria philippinensis, hepatic disease by Capillaria hepatica and pulmonary disease by Capillaria aerophila), Carrion's disease (Bartonella bacilliformis), Cat-scratch disease (Bartonella henselae), Cellulitis (usually Group A Streptococcus and Staphylococcus), Chagas Disease (American trypanosomiasis) (Trypanosoma cruzi), Chancroid (Haemophilus ducreyi), Chickenpox (Varicella zoster virus (VZV)), Chikungunya (Alphavirus), Chlamydia (Chlamydia trachomatis), Chlamydophila pneumoniae infection (Taiwan acute respiratory agent or TWAR) (Chlamydophila pneumoniae), Cholera (Vibrio cholerae), Chromoblastomycosis (usually Fonsecaea pedrosoi), Chytridiomycosis (Batrachochytrium dendrabatidis), Clonorchiasis (Clonorchis sinensis), Clostridium difficile colitis (Clostridium difficile), Coccidioidomycosis (Coccidioides immitis and Coccidioides posadasii), Colorado tick fever (CTF) (Colorado tick fever virus (CTFV)), Common cold (Acute viral rhinopharyngitis; Acute coryza) (usually rhinoviruses and coronaviruses), Coronavirus, Creutzfeldt-Jakob disease (CJD) (PRNP), Crimean-Congo hemorrhagic fever (CCHF) (Crimean-Congo hemorrhagic fever virus), Cryptococcosis (Cryptococcus neoformans), Cryptosporidiosis (Cryptosporidium species), Cutaneous larva migrans (CLM) (usually Ancylostoma braziliense; multiple other parasites), Cyclosporiasis (Cyclospora cayetanensis), Cysticercosis (Taenia solium), Cytomegalovirus infection (Cytomegalovirus), Dengue fever (Dengue viruses (DEN-1, DEN-2, DEN-3 and DEN-4)—Flaviviruses), Desmodesmus infection (Green algae Desmodesmus armatus), Dientamoebiasis (Dientamoeba fragilis), Diphtheria (Corynebacterium diphtheriae), Diphyllobothriasis (Diphyllobothrium), Dracunculiasis (Dracunculus medinensis), Ebola hemorrhagic fever (Ebolavirus (EBOV)), Echinococcosis (Echinococcus species), Ehrlichiosis (Ehrlichia species), Enterobiasis (Pinworm infection) (Enterobius vermicularis), Enterococcus infection (Enterococcus species), Enterovirus infection (Enterovirus species), Epidemic typhus (Rickettsia prowazekii), Erythema infectiosum (Fifth disease) (Parvovirus B19), Exanthem subitum (Sixth disease) (Human herpesvirus 6 (HHV-6) and Human herpesvirus 7 (HHV-7)), Fasciolasis (Fasciola hepatica and Fasciola gigantica), Fasciolopsiasis (Fasciolopsis buski), Fatal familial insomnia (FFI) (PRNP), Filariasis (Filarioidea superfamily), Food poisoning by Clostridium perfringens (Clostridium perfringens), Free-living amebic infection (multiple), Fusobacterium infection (Fusobacterium species), Gas gangrene (Clostridial myonecrosis) (usually Clostridium perfringens; other Clostridium species), Geotrichosis (Geotrichum candidum), Gerstmann-Straussler-Scheinker syndrome (GSS) (PRNP), Giardiasis (Giardia lamblia) Glanders (Burkholderia mallei), Gnathostomiasis (Gnathostoma spinigerum and Gnathostoma hispidum), Gonorrhea (Neisseria gonorrhoeae), Granuloma inguinale (Donovanosis) (Klebsiella granulomatis), Group A streptococcal infection (Streptococcus pyogenes), Group B streptococcal infection (Streptococcus agalactiae), Haemophilus influenzae infection (Haemophilus influenzae) Hand, foot and mouth disease (HFMD) (Enteroviruses, mainly Coxsackie A virus and Enterovirus 71 (EV71)), Hantavirus Pulmonary Syndrome (HPS) (Sin Nombre virus), Heartland virus disease (Heartland virus), Helicobacter pylori infection (Helicobacter pylori), Hemolytic-uremic syndrome (HUS), Escherichia coli O157:H7, O111 and O104:H4, Hemorrhagic fever with renal syndrome (HFRS) (Bunyaviridae family), Hepatitis A (Hepatitis A virus), Hepatitis B (Hepatitis B virus), Hepatitis C (Hepatitis C virus), Hepatitis D (Hepatitis D Virus), Hepatitis E (Hepatitis E virus), Herpes simplex (Herpes simplex virus 1 and 2 (HSV-1 and HSV-2)), Histoplasmosis (Histoplasma capsulatum), Hookworm infection (Ancylostoma duodenale and Necator americanus), Human bocavirus infection (Human bocavirus (HBoV)), Human ewingii ehrlichiosis (Ehrlichia ewingii), Human granulocytic anaplasmosis (HGA) (Anaplasma phagocytophilum), Human metapneumovirus infection, Human metapneumovirus (hMPV), Human monocytic ehrlichiosis (Ehrlichia chaffeensis), Human papillomavirus (HPV) infection (Human papillomavirus (HPV)), Human parainfluenza virus infection (Human parainfluenza viruses (HPIV)), Hymenolepiasis (Hymenolepis nana and Hymenolepis diminuta), Epstein-Barr virus infectious mononucleosis (Mono) (Epstein-Barr virus (EBV)), Influenza (flu) (Orthomyxoviridae family) Isosporiasis (Isospora belli), Kawasaki disease (unknown; evidence supports that it is infectious) Keratitis (multiple), Kingella kingae infection (Kingella kingae), Kuru (PRNP), Lassa fever (Lassa virus), Legionellosis (Legionnaires' disease) (Legionella pneumophila), Legionellosis (Pontiac fever) (Legionella pneumophila), Leishmaniasis (Leishmania species), Leprosy (Mycobacterium leprae and Mycobacterium lepromatosis), Leptospirosis (Leptospira species), Listeriosis (Listeria monocytogenes), Lyme disease (Lyme borreliosis) (Borrelia burgdorferi, Borrelia garinii, and Borrelia afzelii), Lymphatic filariasis (Elephantiasis) (Wuchereria bancrofti and Brugia malayi), Lymphocytic choriomeningitis (Lymphocytic choriomeningitis virus (LCMV)), Malaria (Plasmodium species), Marburg hemorrhagic fever (MHF) (Marburg virus), Measles (Measles virus), Middle East respiratory syndrome (MERS) (Middle East respiratory syndrome coronavirus), Melioidosis (Whitmore's disease) (Burkholderia pseudomallei), Meningitis (multiple), Meningococcal disease (Neisseria meningitidis), Metagonimiasis (usually Metagonimus yokagawai), Microsporidiosis (Microsporidia phylum), Molluscum contagiosum (MC) (Molluscum contagiosum virus (MCV)), Monkeypox (Monkeypox virus), Mumps (Mumps virus), Murine typhus (Endemic typhus) (Rickettsia typhi), Mycoplasma pneumonia (Mycoplasma pneumoniae), Mycetoma (disambiguation) (numerous species of bacteria (Actinomycetoma) and fungi (Eumycetoma)), Myiasis (parasitic dipterous fly larvae), Neonatal conjunctivitis (Ophthalmia neonatorum) (most commonly Chlamydia trachomatis and Neisseria gonorrhoeae), Norovirus (children and babies) ((New) Variant Creutzfeldt-Jakob disease (vCJD, nvCJD), PRNP), Nocardiosis (usually Nocardia asteroides and other Nocardia species), Onchocerciasis (River blindness) (Onchocerca volvulus), Opisthorchiasis (Opisthorchis viverrini and Opisthorchis felineus), Paracoccidioidomycosis (South American blastomycosis) (Paracoccidioides brasiliensis), Paragonimiasis (usually Paragonimus westermani and other Paragonimus species), Pasteurellosis (Pasteurella species), Pediculosis capitis (Head lice) (Pediculus humanus capitis), Pediculosis corporis (Body lice) (Pediculus humanus corporis), Pediculosis pubis (Pubic lice, Crab lice) (Phthirus pubis), Pelvic inflammatory disease (PID) (multiple), Pertussis (Whooping cough) (Bordetella pertussis), Plague (Yersinia pestis), Pneumococcal infection (Streptococcus pneumoniae), Pneumocystis pneumonia (PCP) (Pneumocystis jirovecii), Pneumonia (multiple), Poliomyelitis (Poliovirus), Prevotella infection (Prevotella species), Primary amoebic meningoencephalitis (PAM) (usually Naegleria fowleri), Progressive multifocal leukoencephalopathy (JC virus), Psittacosis (Chlamydophila psittaci), Q fever (Coxiella burnetii), Rabies (Rabies virus), Relapsing fever (Borrelia hermsii, Borrelia recurrentis, and other Borrelia species), Respiratory syncytial virus infection (Respiratory syncytial virus (RSV)), Rhinosporidiosis (Rhinosporidium seeberi), Rhinovirus infection (Rhinovirus), Rickettsial infection (Rickettsia species), Rickettsialpox (Rickettsia akari), Rift Valley fever (RVF) (Rift Valley fever virus), Rocky Mountain spotted fever (RMSF) (Rickettsia rickettsii), Rotavirus infection (Rotavirus), Rubella (Rubella virus), Salmonellosis (Salmonella species), SARS (Severe Acute Respiratory Syndrome) (SARS coronavirus), Scabies (Sarcoptes scabiei), Schistosomiasis (Schistosoma species), Sepsis (multiple), Shigellosis (Bacillary dysentery) (Shigella species), Shingles (Herpes zoster) (Varicella zoster virus (VZV)), Smallpox (Variola) (Variola major or Variola minor), Sporotrichosis (Sporothrix schenckii), Staphylococcal food poisoning (Staphylococcus species), Staphylococcal infection (Staphylococcus species), Strongyloidiasis (Strongyloides stercoralis), Subacute sclerosing panencephalitis (Measles virus), Syphilis (Treponema pallidum), Taeniasis (Taenia species), Tetanus (Lockjaw) (Clostridium tetani), Tinea barbae (Barber's itch) (usually Trichophyton species), Tinea capitis (Ringworm of the Scalp) (usually Trichophyton tonsurans), Tinea corporis (Ringworm of the Body) (usually Trichophyton species), Tinea cruris (Jock itch) (usually Epidermophyton floccosum, Trichophyton rubrum, and Trichophyton mentagrophytes), Tinea manum (Ringworm of the Hand) (Trichophyton rubrum), Tinea nigra (usually Hortaea werneckii), Tinea pedis (Athlete's foot) (usually Trichophyton species), Tinea unguium (Onychomycosis) (usually Trichophyton species), Tinea versicolor (Pityriasis versicolor) (Malassezia species), Toxocariasis (Ocular Larva Migrans (OLM)) (Toxocara canis or Toxocara cati), Toxocariasis (Visceral Larva Migrans (VLM)) (Toxocara canis or Toxocara cati), Trachoma (Chlamydia trachomatis), Toxoplasmosis (Toxoplasma gondii), Trichinosis (Trichinella spiralis), Trichomoniasis (Trichomonas vaginalis), Trichuriasis (Whipworm infection) (Trichuris trichiura), Tuberculosis (usually Mycobacterium tuberculosis), Tularemia (Francisella tularensis), Typhoid fever (Salmonella enterica subsp. enterica, serovar typhi), Typhus fever (Rickettsia), Ureaplasma urealyticum infection (Ureaplasma urealyticum), Valley fever (Coccidioides immitis or Coccidioides posadasii), Venezuelan equine encephalitis (Venezuelan equine encephalitis virus), Venezuelan hemorrhagic fever (Guanarito virus), Vibrio vulnificus infection (Vibrio vulnificus), Vibrio parahaemolyticus enteritis (Vibrio parahaemolyticus), Viral pneumonia (multiple viruses), West Nile Fever (West Nile virus), White piedra (Tinea blanca) (Trichosporon beigelii), Yersinia pseudotuberculosis infection (Yersinia pseudotuberculosis), Yersiniosis (Yersinia enterocolitica), Yellow fever (Yellow fever virus), Zygomycosis (Mucorales order (Mucormycosis) and Entomophthorales order (Entomophthoramycosis)) Human immunodeficiency virus [HIV] disease, HIV disease with infectious and parasitic diseases, HIV disease with mycobacterial infection, HIV disease with cytomegaloviral disease, HIV disease with other viral infections, HIV disease with candidiasis, HIV disease with other mycoses, HIV disease with Pneumocystic carinii pneumonia, HIV disease with malignant neoplasms, HIV disease with Kaposi's sarcoma, HIV disease with Burkitt's lymphoma, HIV disease with other type's of non-Hodgkin's lymphoma, HIV disease with other malignant neoplasms of lymphoid, hematopoietic and related tissue, HIV disease with multiple malignant neoplasms, HIV disease with other malignant neoplasms, HIV disease with unspecified malignant neoplasm, HIV disease with encephalopathy, HIV disease with lymphoid interstitial pneumonitis, HIV disease with wasting syndrome, HIV disease with multiple diseases classified elsewhere, HIV disease with other conditions, HIV disease Acute HIV infection syndrome, HIV disease with (persistent) generalized lymphadenopathy, HIV disease with hematological and immunological abnormalities, HIV disease with other specified conditions, or Unspecified HIV disease. In some embodiments of the disclosure the infectious disease may be infection with a virus, such as a virus from one of the following families of viruses: a) Adenoviridae family, Such as Adenovirus species; b) Herpesviridae family, Such as Herpes simplex type 1, Herpes simplex type 2, Varicella Zoster virus, Epstein-barr virus, Human cytomegalovirus, Human herpesvirus type 8 species; c) Papillomaviridae fam ily, Such as Human papillomavirus species; d) Polyomaviri dae family, such as BK virus, JC virus species; e) Poxviridae family, Such as Smallpox species: f) Hepadnaviridae family, such as Hepatitis B virus species: g) Parvoviridae family, such as Human bocavirus, Parvovirus B19 species; h) Astroviridae family, such as Human astrovirus species: i) Caliciviridae family, such as Norwalk virus species; j) Flaviviridae family, such as Hepatitis C virus (HCV), yellow fever virus, dengue virus, West Nile virus species; k) Togaviridae family, such as Rubella virus species; 1) Hepeviridae family, such as Hepatitis E virus species; m) Retroviridae family, such as Human immunodeficiency virus (HIV) species; n) Orthomyxoviri daw family, such as Influenza virus species; o) Arenaviridae family, such as Guanarito virus, Junin virus, Lassa virus, Machupo virus, and/or Sabiá virus species; p) Bunyaviridae family, Such as Crimean-Congo hemorrhagic fever virus species; q) Filoviridae family, such as Ebola virus and/or Marburg virus species; Paramyxoviridae family, Such as Measles virus, Mumps virus, Parainfluenza virus, Respiratory syncy tial virus, Human metapneumovirus, Hendra virus and/or Nipah virus species; r) Rhabdoviridae genus, such as Rabies virus species; s) Reoviridae family, such as Rotavirus, Orbivirus, Coltivirus and/or Banna virus species.

In some embodiments of the disclosure, the infectious disease may not be one of the above recited infectious diseases.

In some embodiments, the infectious disease may be a disease caused by infection with an influenza A (Flu A) virus. In some embodiments the influenza virus can be an avian or swine-origin pandemic influenza virus, for example, H5N1, H7N3, H7N7, H7N9 and H9N2 (avian subtypes) or H1N1, H1N2, H2N1, H3N1, H3N2, or H2N3 (swine subtypes).

In some preferred embodiments of the disclosure, the infectious disease may be HIV, such as residual HIV disease, herpes, hepatitis or human papilloma virus. In other preferred embodiments, the infectious disease may be a disease resulting from infection with a coronavirus, for example COVID-19 (coronavirus 2019; the disease caused by severe acute respiratory syndrome coronavirus 2, SARS-CoV-2).

In embodiments in which the methods of the disclosure are carried out on a subject having, suspected of having, or having been diagnosed with infectious disease, the NKT cells, T cells, and/or dendritic cells produced by these methods may treat the infectious disease. In this context, “treat” means to exert a beneficial therapeutic effect in the subject, which can be any overall clinical benefit derived from the methods of the disclosure. This overall clinical benefit can be any of, for example: reduced fever, reduced diarrhea, reduced coughing, reduced muscle aches, reduced fatigue, reduced CRP, reduced time on ventilator, reduced need for extra oxygen, reduced organ damage after recovery.

In some embodiments, the NKT cells of the disclosure may treat the infectious disease via engulfing and killing the infectious organism, activating other innate and adaptive immune cells, recruiting other immune cells to the site of infection (e.g. an organ infected by a virus), depleting immune cells infected by the virus (e.g. monocytes activated by COVID-19).

In some embodiments, the T cells of the disclosure may treat the infectious disease via release of immune activating cytokines. In some embodiments, the T cells of the disclosure may treat the infectious disease via release of cytokines having anti-microbial or anti-viral effects (for example, TNF-alpha, IFN-gamma). In some embodiments, the T cells of the disclosure may treat the infectious disease by inducing apoptosis, for example by expressing ligands which engage death receptors on the target cells. In some embodiments, the T cells may secrete cytotoxic molecules which kill the infectious organism. In some embodiments, the T cells of the disclosure may ingest or engulf the infectious organism.

In some embodiments, the dendritic cells of the disclosure may treat the infectious disease by conveying pathogen-associated signals to the adaptive branch of the immune system. In some embodiments, the dendritic cells of the disclosure may treat the infectious disease by promoting T cell infiltration to the site of infection and/or by priming cytotoxic T cells to kill the infectious organism.

In embodiments in which the infectious disease is a disease resulting from infection with a coronavirus, for example COVID-19, the NKT cells of the disclosure may treat the disease via engulfing and killing the coronavirus, and/or by activating other innate and adaptive immune cells.

Thus, the present disclosure also provides methods of treating a disease resulting from infection with a coronavirus in a subject, the method comprising administering a glucocorticoid-receptor (GR) modulating agent to the subject at a dose equivalent to about at least 6 mg/kg human equivalent dose (HED) of dexamethasone base. In some embodiments, the glucocorticoid-receptor (GR) modulating agent may be a glucocorticoid, preferably dexamethasone or betamethasone. In some embodiments, the glucocorticoid-receptor (GR) modulating agent may be administered at a dose equivalent to about at least 15 mg/kg human equivalent dose (HED) of dexamethasone base. In some preferred embodiments, the glucocorticoid-receptor (GR) modulating agent may be administered at a dose equivalent to between about 18 mg/kg and 30 mg/kg human equivalent dose (HED) of dexamethasone base. In some preferred embodiments, the disease is COVID-19 (coronavirus 2019; the disease caused by severe acute respiratory syndrome coronavirus 2, SARS-CoV-2) or SARS-CoV or MERS. In some embodiments, the glucocorticoid-receptor (GR) modulating agent induces a population of NKT cells and/or T cells as disclosed elsewhere herein. In some embodiments, the glucocorticoid-receptor (GR) modulating agent activates a population of dendritic cells as disclosed elsewhere herein.

In some preferred embodiments, the present disclosure provides a method of treating COVID-19 (coronavirus 2019; the disease caused by severe acute respiratory syndrome coronavirus 2, SARS-CoV-2) in a subject, the method comprising administering dexamethasone or betamethasone to the subject at a dose equivalent to between about 15 mg/kg and 30 mg/kg human equivalent dose (HED) of dexamethasone base.

In embodiments in which the infectious disease is a disease resulting from infection with a coronavirus, for example COVID-19, the glucocorticoid receptor modulating agent may be administered in combination with a proton pump inhibitor (such as omeprazole) and/or hydrocortisone. In this context, “in combination with” may mean concurrent administration or may mean separate and/or sequential administration in any order.

In some embodiments of the methods of the disclosure, the methods of producing/mobilizing a population of natural killer T cells (NKT cells), producing/mobilizing a population of T cells, and/or mobilizing/activating a population of dendritic cells may further comprise a step of isolating an NKT cell, T cell, and/or dendritic cell, or a population of NKT cells, T cells, and/or dendritic cells from the subject or from a sample derived from the subject. Accordingly, the present disclosure provides isolated NKT cells isolated T cells, and isolated dendritic cells, as well as isolated populations of NKT cells, T cells, and dendritic cells. The isolated cells and isolated populations of cells may be characterized by the pattern of surface proteins which they express, as outlined above.

Suitable methods for isolating cells and populations of cells from a mixed sample are well-known to the skilled person—for example, flow sorting (such as fluorescence-activated cell sorting; FACS) and magnetic particle sorting (such as magnetic-activated cell sorting; MACS), microfluidic cell sorting, density gradient centrifugation, immunodensity cell isolation, expansion in cell culture based on growth factors and other components in the media. In some preferred embodiments of the disclosure, the step of isolating is performed by fluorescence-activated cell sorting (FACS) or magnetic-activated cell sorting (MACS).

In embodiments in which the NKT cells, T cells, and/or dendritic cells are isolated from a sample derived from the subject, the sample may be selected from the group consisting of: blood, plasma, a tumor biopsy or surgically removed tumor, bone marrow, liver, spleen biopsy, and fat or adipose tissue.

In some embodiments, the step of isolating may be performed at least about 1, 3, 12, 24, 48, 72, 96, 120, 144, or 168 hours after administration of glucocorticoid-receptor (GR) modulating agent or ICAM3 modulating agent. In some embodiments, the step of isolating may be performed at least about 1, 3, 8, 9, 10, 11, 12, 13, 14, or 15 days after administration of glucocorticoid-receptor (GR) modulating agent or ICAM3 modulating agent. In some preferred embodiments, the step of isolating is performed at least about 48 hours after said administration. In some other preferred embodiments, the step of isolating is performed at about 1, 3, or 48 hours after said administration. In some embodiments, the step of isolating may be performed between about 1, 3, or 48 hours and 13 days, between about 1, 3, or 48 hours and 168 hours, between about 1, 3, or 48 hours and 120 hours, between about 1, 3, or 48 hours and 96 hours, or between about 1, 3, or 48 hours and 72 hours after administration of glucocorticoid-receptor (GR) modulating agent or ICAM3 modulating agent. In some preferred embodiments, the step of isolating is performed between about 1, 3, or 48 hours and 72 hours after said administration. In some embodiments the step of isolating may be performed within 1, 2, 3, 4, 5, 6, 7, 8, 9, or 10 hours after glucocorticoid administration. In some preferred embodiments, the step of isolating may be performed within 3 hours after glucocorticoid administration. In some particularly preferred embodiments the step of isolating may be performed within 1 hour after glucocorticoid administration. In some preferred embodiments in which the subject has cancer, an infectious disease, or autoimmune disease, the step of isolating the NKT cells may be performed on a blood sample from the subject, within 3 hours after glucocorticoid administration, and preferably within 1 hour after glucocorticoid administration.

In some preferred embodiments of methods involving a step of isolating, the subject may be a healthy subject, such as a healthy adult human subject. In this context a healthy subject is a subject which is not afflicted with disease.

The isolated NKT cells, T cells, and/or dendritic cells, and the isolated NKT cell populations, T cell populations, and/or dendritic cell populations of the disclosure can be expanded in culture. Suitable methods and reagents for culturing and expanding cells are well-known to the skilled person. For instance, long term culture with IL-2, soluble anti-CD28 antibody, anti-CD3 epsilon antibody, anti-TCRbeta antibody, and glycolipids such as KRN7000, PB S44, or PBS57 has been shown to produce robust expansion of NKT cells (Watarai et al 2008, which is hereby incorporated by reference in its entirety). Accordingly, in some embodiments of the methods of the disclosure, the method of producing a population of natural killer T cells (NKT cells), producing a population of T cells, and/or activating a population of dendritic cells may further comprise a step of expanding the NKT cell, T cell, dendritic cell or NKT cells, T cells, or dendritic cells isolated by the step of isolating. In some embodiments of the method of the disclosure, the method may further comprise a step of activating the isolated cells (either before or after the step of expanding) with an NKT cell activator, T cell activator, or dendritic cell activator, which may be as described in detail above.

In some embodiments, following isolation of an NKT cell, T cell, or dendritic cell or population of NKT cells, population of T cells, or population of dendritic cells from the subject or from a sample derived from the subject, the methods of the disclosure may further comprise a step of introducing a nucleic acid encoding a protein into the isolated cell or cells. Suitable methods for introducing a nucleic acid into a cell are well known to the skilled person—for example, physical or chemical methods including electroporation, sonoporation, cell microinjection, microparticle delivery, calcium-phosphate mediated transfection, and liposome-based transfection; or, viral transduction. Following introduction of the nucleic acid encoding a protein, the cell or cells may be cultured under conditions that facilitate expression of the encoded protein. Suitable methods, reagents, and conditions for culturing cells are well-known to the skilled person. The cell (NKT cell, T cell, or dendritic cell) or cells (NKT cells, T cells, or dendritic cells) into which a nucleic acid encoding a protein has been introduced may be referred to herein as transfected or transformed cells.

In some embodiments of the disclosure, the nucleic acid encoding a protein is a nucleic acid which encodes a protein selected from the group consisting of one or more of: a T-cell receptor (TCR), a chimeric antigen receptor (CAR), a split, and universal and programmable CAR (SUPRA-CAR).

After isolation, the NKT cells, T cells, and/or dendritic cells may be genetically engineered for a particular target. For example, the NKT can be expanded by IL-2 and activated with GalCer (galactosylceramide), pulsed autologous irradiated PBMCs, then transduced to express a CAR or recombinant TCR (rTCR). The CAR or rTCR may specifically bind a target selected from GD2 (disialoganglioside) and CD19. For instance, the CAR may be NCT03294954 (which specifically binds GD2) or NCT03774654 (which specifically binds CD19).

Moreover, the NKT cells, T cells, and/or dendritic cells can undergo targeted activation. For instance, the following procedures can be utilized: Nanovectors for passive and active delivery; a-GalCer-loaded APCs for targeted activation of NKT to tumors; i.v. administration of a-GalCer; and/or bulk PBMCs stimulation (two to three times) via addition of a-GalCer to the cultured cells (to produce an iNKT cell-enriched population, which is then infused back into the patient)

Moreover, the NKT cells, T cells, and/or dendritic cells can be directly linked to tumor targeting moieties (either on tumor cells or TME). Chemical modification of stimulatory agents for NKT cells (polarization of immune responses by a-GalCer analogues), T cells, and dendritic cells can also be employed.

The term “chimeric antigen receptor” (CAR) as used herein non-exclusively relates to constructs that contain an antigen-binding domain of an antibody fused to a strong T-cell activator domain. T-cells modified with the CAR construct can bind to the antigen and be stimulated to attack the bound cells. Artificial T cell receptors (also known as chimeric T cell receptors, chimeric immunoreceptors, chimeric antigen receptors (CARs)) are engineered receptors, which graft an arbitrary specificity onto an immune effector cell. The receptors are called chimeric because they are composed of parts from different sources. The receptor/ligand or antibody expressed by the chimeric antigen receptor T cells or cellular immunotherapy can be mono- or bi-specific or multi-specific.

In some embodiments, the TCR, CAR, and/or SUPRA-CAR may comprise an antigen-binding domain which binds to an antigen selected from the group of receptors/ligands/targets consisting of: Proto-oncogene tyrosine-protein kinase ABL1, Citrullinated Antigen, ErbB2/HER2, CD16, WT-1, KRAS, glypican 3, CD3, CD20, CD226, CD155, CD123, HPV-16 E6, Melan-A/MART-1, TRAIL Bound to the DR4 Receptor, LMP, MTCR, ESO, NY-ESO-1, gp100, 4SCAR-GD2/CD56, Mesothelin (CAK1 Antigen or Pre Pro Megakaryocyte Potentiating Factor or MSLN); DNA Synthesis Inhibitor; Histamine H1 Receptor (HRH1) Antagonist; Prostaglandin G/H Synthase 2 (Cyclooxygenase 2 or COX2 or Prostaglandin Endoperoxide Synthase 2 or PHS II or Prostaglandin H2 Synthase 2 or PTGS2 or EC 1.14.99.1) Inhibitor, CD19 (B Lymphocyte Surface Antigen B4 or Differentiation Antigen CD19 or T Cell Surface Antigen Leu 12 or CD19), Cell Adhesion Molecule 5 (Carcinoembryonic Antigen or CEA or Meconium Antigen 100 or CD66e or CEACAM5); Interleukin 2 Receptor (IL2R) Agonist, Epidermal Growth Factor Receptor (Proto Oncogene c ErbB 1 or Receptor Tyrosine Protein Kinase erbB 1 or HER1 or ERBB1 or EGFR or EC 2.7.10.1); DNA Ligase (EC 6.5.1.) Inhibitor; DNA Ligase (EC 6.5.1.), DNA Polymerase Alpha (POLA or EC 2.7.7.7) Inhibitor; DNA Primase (EC 2.7.7.6) Inhibitor; Ribonucleoside Diphosphate Reductase (Ribonucleotide Reductase or RRM or EC 1.17.4.1) Inhibitor; RNA Polymerase II (RNAP II or Pol II or EC 2.7.7.6) Inhibitor, DNA Polymerase (EC 2.7.7.7) Inhibitor; DNA Topoisomerase II (EC 5.99.1.3) Inhibitor; CD22, meso, DNA Primase (EC 2.7.7.6); Programmed Cell Death 1 Ligand 1 (PD L1 or B7 Homolog 1 or CD274) Inhibitor; RNA Polymerase II (RNAP II or Pol II or EC 2.7.7.6), Histone Lysine N Methyltransferase EZH2 (ENX 1 or Enhancer Of Zeste Homolog 2 or Lysine N Methyltransferase 6 or EZH2 or EC 2.1.1.43) Inhibitor; Programmed Cell Death 1 Ligand 1 (PD L1 or B7 Homolog 1 or CD274), C—X—C Chemokine Receptor Type 4 (FB22 or Fusin or HM89 or LCR1 or Leukocyte Derived Seven Transmembrane Domain Receptor or Lipopolysaccharide Associated Protein 3 or Stromal Cell Derived Factor 1 Receptor or NPYRL or CD184 or CXCR4) Antagonist; Granulocyte Colony Stimulating Factor Receptor (CD114 or GCSFR or CSF3R) Agonist, Adenosine Deaminase (Adenosine Aminohydrolase or ADA or EC 3.5.4.4) Inhibitor; Tumor Necrosis Factor Receptor Superfamily Member 17 (B Cell Maturation Antigen or CD269 or TNFRSF17), Cytocytotoxic To Cells Expressing Inactive Tyrosine Protein Kinase Transmembrane Receptor ROR1 (Neurotrophic Tyrosine Kinase Receptor Related 1 or ROR1 or EC 2.7.10.1); T Cell Surface Glycoprotein CD3 Epsilon Chain (T Cell Surface Antigen T3/Leu 4 Epsilon Chain or CD3E); Dihydrofolate Reductase (DHFR or EC 1.5.1.3) Inhibitor; Ephrin Type A Receptor 2 (Epithelial Cell Kinase or Tyrosine Protein Kinase Receptor ECK or EPHA2 or EC 2.7.10.1) Inhibitor; Glucocorticoid Receptor (GR or Nuclear Receptor Subfamily 3 Group C Member 1 or NR3C1) Agonist; Mast/Stem Cell Growth Factor Receptor Kit (Proto Oncogene c Kit or Tyrosine Protein Kinase Kit or v Kit Hardy Zuckerman 4 Feline Sarcoma Viral Oncogene Homolog or Piebald Trait Protein or p145 c Kit or CD117 or KIT or EC 2.7.10.1) Inhibitor; Platelet Derived Growth Factor Receptor Beta (Beta Type Platelet Derived Growth Factor Receptor or CD140 Antigen Like Family Member B or Platelet Derived Growth Factor Receptor 1 or CD140b or PDGFRB or EC 2.7.10.1) Inhibitor; Tubulin Inhibitor; Tyrosine Protein Kinase CSK (C Src Kinase or Protein Tyrosine Kinase CYL or CSK or EC 2.7.10.2) Inhibitor; Tyrosine Protein Kinase Fyn (Proto Oncogene Syn or Proto Oncogene c Fyn or Src Like Kinase or p59 Fyn or FYN or EC 2.7.10.2) Inhibitor; Tyrosine Protein Kinase Lck (Leukocyte C Terminal Src Kinase or Protein YT16 or Proto Oncogene Lck or T Cell Specific Protein Tyrosine Kinase or Lymphocyte Cell Specific Protein Tyrosine Kinase or p56 LCK or LCK or EC 2.7.10.2) Inhibitor; Tyrosine Protein Kinase Yes (Proto Oncogene c Yes or p61 Yes or YES1 or EC 2.7.10.2) Inhibitor, Tumor Necrosis Factor (Cachectin or TNF Alpha or Tumor Necrosis Factor Ligand Superfamily Member 2 or TNF a or TNF) Inhibitor, Signal Transducer And Activator Of Transcription 3 (Acute Phase Response Factor or DNA Binding Protein APRF or STAT3) Inhibitor, Bcr-Abl Tyrosine Kinase (EC 2.7.10.2) Inhibitor; Dihydrofolate Reductase (DHFR or EC 1.5.1.3); Ephrin Type A Receptor 2 (Epithelial Cell Kinase or Tyrosine Protein Kinase Receptor ECK or EPHA2 or EC 2.7.10.1); Mast/Stem Cell Growth Factor Receptor Kit (Proto Oncogene c Kit or Tyrosine Protein Kinase Kit or v Kit Hardy Zuckerman 4 Feline Sarcoma Viral Oncogene Homolog or Piebald Trait Protein or p145 c Kit or CD117 or KIT or EC 2.7.10.1); Platelet Derived Growth Factor Receptor Beta (Beta Type Platelet Derived Growth Factor Receptor or CD140 Antigen Like Family Member B or Platelet Derived Growth Factor Receptor 1 or CD140b or PDGFRB or EC 2.7.10.1); Tubulin; Tyrosine Protein Kinase CSK (C Src Kinase or Protein Tyrosine Kinase CYL or CSK or EC 2.7.10.2) Inhibitor; Tyrosine Protein Kinase Fyn (Proto Oncogene Syn or Proto Oncogene c Fyn or Src Like Kinase or p59 Fyn or FYN or EC 2.7.10.2) Inhibitor; Tyrosine Protein Kinase Lck (Leukocyte C Terminal Src Kinase or Protein YT16 or Proto Oncogene Lck or T Cell Specific Protein Tyrosine Kinase or Lymphocyte Cell Specific Protein Tyrosine Kinase or p56 LCK or LCK or EC 2.7.10.2) Inhibitor; Tyrosine Protein Kinase Yes (Proto Oncogene c Yes or p61 Yes or YES1 or EC 2.7.10.2) Inhibitor, Caspase 9 (Apoptotic Protease Mch 6 or Apoptotic Protease Activating Factor 3 or ICE Like Apoptotic Protease 6 or CASP9 or EC 3.4.22.62) Activator; Prostate Stem Cell Antigen (PSCA), Melanoma Antigen Preferentially Expressed In Tumors (Cancer/Testis Antigen 130 or Opa Interacting Protein 4 or OIP4 or Preferentially Expressed Antigen Of Melanoma or PRAME), Signal Transducer And Activator Of Transcription 3 (Acute Phase Response Factor or DNA Binding Protein APRF or STAT3) Inhibitor, CD44 Antigen (CDw44 or Epican or Extracellular Matrix Receptor III or GP90 Lymphocyte Homing/Adhesion Receptor or HUTCH I or Heparan Sulfate Proteoglycan or Hermes Antigen or Hyaluronate Receptor or Phagocytic Glycoprotein 1 or CD44), AXL (anexelekto) receptor tyrosine kinase, GAS6, TAM receptor tyrosine kinases, TYRO-3 (also known as Brt, Dtk, Rse, Sky and Tif), AXL (also known as Ark, Tyrol and Ufo), and MER (also known as Eyk, Nym and Tyro12), CTLA4, Tumor Necrosis Factor Receptor Superfamily Member 8 (CD30L Receptor or Ki 1 Antigen or Lymphocyte Activation Antigen CD30 or CD30 or TNFRSF8), Caspase 9 (Apoptotic Protease Mch 6 or Apoptotic Protease Activating Factor 3 or ICE Like Apoptotic Protease 6 or CASP9 or EC 3.4.22.62) Activator; Cytocytotoxic To Cells Expressing Ganglioside GD2; Prostaglandin G/H Synthase 1 (Cyclooxygenase 1 or COX1 or Prostaglandin Endoperoxide Synthase 1 or Prostaglandin H2 Synthase 1 or PTGS1 or EC 1.14.99.1) Inhibitor; cytokines, interleukins, Claudin 6 (Skullin or CLDN6), NKG2D, MICA, MICB and ULBP 1-6, NKp30, B7H6 (NCR3LG1), Bag6, B7 family, CD40 Ligand (T Cell Antigen Gp39 or TNF Related Activation Protein or Tumor Necrosis Factor Ligand Superfamily Member 5 or CD154 or CD40LG) Activator; Interleukin 12 (IL12) Activator, Interleukin 3 Receptor Subunit Alpha (IL3RAMast/Stem Cell Growth F), actor Receptor Kit (Proto Oncogene c Kit or Tyrosine Protein Kinase Kit or v Kit Hardy Zuckerman 4 Feline Sarcoma Viral Oncogene Homolog or Piebald Trait Protein or p145 c Kit or CD117 or KIT or EC 2.7.10.1) Antagonist; Proto Oncogene Tyrosine Protein Kinase Receptor Ret (Cadherin Family Member 12 or Proto Oncogene c Ret or RET or EC 2.7.10.1) Inhibitor; Receptor Type Tyrosine Protein Kinase FLT3 (FMS Like Tyrosine Kinase 3 or FL Cytokine Receptor or Stem Cell Tyrosine Kinase 1 or Fetal Liver Kinase 2 or CD135 or FLT3 or EC 2.7.10.1) Antagonist; Vascular Endothelial Growth Factor Receptor 1 (Fms Like Tyrosine Kinase 1 or Tyrosine Protein Kinase Receptor FLT or Tyrosine Protein Kinase FRT or Vascular Permeability Factor Receptor or VEGFR1 or FLT1 or EC 2.7.10.1) Antagonist; Vascular Endothelial Growth Factor Receptor 2 (Fetal Liver Kinase 1 or Kinase Insert Domain Receptor or Protein Tyrosine Kinase Receptor flk 1 or VEGFR2 or CD309 or KDR or EC 2.7.10.1) Antagonist; Vascular Endothelial Growth Factor Receptor 3 (Fms Like Tyrosine Kinase 4 or Tyrosine Protein Kinase Receptor FLT4 or VEGFR3 or FLT4 or EC 2.7.10.1) Antagonist, Caspase 9 (Apoptotic Protease Mch 6 or Apoptotic Protease Activating Factor 3 or ICE Like Apoptotic Protease 6 or CASP9 or EC 3.4.22.62) Activator, Cytocytotoxic T Lymphocyte Protein 4 (Cytocytotoxic T Lymphocyte Associated Antigen 4 or CD152 or CTLA4) Antagonist, Myeloid Cell Surface Antigen CD33 (Sialic Acid Binding Ig Like Lectin 3 or gp67 or CD33), Hepatocyte Growth Factor Receptor (Proto Oncogene c Met or Tyrosine Protein Kinase Met or HGF/SF Receptor or Scatter Factor Receptor or MET or EC 2.7.10.1), Epithelial Cell Adhesion Molecule (Adenocarcinoma Associated Antigen or Cell Surface Glycoprotein Trop 1 or Epithelial Cell Surface Antigen or Epithelial Glycoprotein 314 or KS 1/4 Antigen or KSA or Tumor Associated Calcium Signal Transducer 1 or CD326 or EPCAM), Ganglioside GD2, Lewis Y Antigen (CD174), Latent Membrane Protein 1 (Protein p63 or LMP1), Mucin 1 (Breast Carcinoma Associated Antigen DF3 or Episialin or H23AG or Krebs Von Den Lungen 6 or PEMT or Peanut Reactive Urinary Mucin or Polymorphic Epithelial Mucin or Tumor Associated Epithelial Membrane Antigen or Tumor Associated Mucin or CD227 or MUC1), T Cell Receptor Beta 1 Chain C Region (TRBC1), Vascular Endothelial Growth Factor Receptor 2 (Fetal Liver Kinase 1 or Kinase Insert Domain Receptor or Protein Tyrosine Kinase Receptor flk 1 or VEGFR2 or CD309 or KDR or EC 2.7.10.1), BCMA, PD-1, interleukin-6 receptor, NKR2, CX-072, T Lymphocyte Protein 4 (Cytocytotoxic T Lymphocyte Associated Antigen 4 or CD152 or CTLA4) Antagonist; Serine/Threonine Protein Kinase B Raf (p94 or Proto Oncogene B Raf or v Raf Murine Sarcoma Viral Oncogene Homolog B1 or BRAF or EC 2.7.11.1) Inhibitor, Mucin 16 (Ovarian Cancer Related Tumor Marker CA125 or Ovarian Carcinoma Antigen CA125 or MUC16); Bcr-Abl Tyrosine Kinase (EC 2.7.10.2) Inhibitor; Tyrosine Protein Kinase CSK (C Src Kinase or Protein Tyrosine Kinase CYL or CSK or EC 2.7.10.2) Inhibitor; Tyrosine Protein Kinase Fyn (Proto Oncogene Syn or Proto Oncogene c Fyn or Src Like Kinase or p59 Fyn or FYN or EC 2.7.10.2) Inhibitor; Tyrosine Protein Kinase Lck (Leukocyte C Terminal Src Kinase or Protein YT16 or Proto Oncogene Lck or T Cell Specific Protein Tyrosine Kinase or Lymphocyte Cell Specific Protein Tyrosine Kinase or p56 LCK or LCK or EC 2.7.10.2) Inhibitor; Tyrosine Protein Kinase Yes (Proto Oncogene c Yes or p61 Yes or YES1 or EC 2.7.10.2) Inhibitor, Cyclin Dependent Kinase 1 (p34 Protein Kinase or Cell Division Protein Kinase 1 or Cell Division Control Protein 2 Homolog or CDK1 or EC 2.7.11.22 or EC 2.7.11.23) Inhibitor; Cyclin Dependent Kinase 2 (p33 Protein Kinase or Cell Division Protein Kinase 2 or CDK2 or EC 2.7.11.22) Inhibitor; Granulocyte Macrophage Colony Stimulating Factor Receptor Subunit Alpha (CDw116 or CD116 or CSF2RA) Agonist, EGFRVIII, Tyrosine Protein Kinase SYK (Spleen Tyrosine Kinase or p72 Syk or SYK or EC 2.7.10.2) Inhibitor, Alpha Fetoprotein (Alpha 1 Fetoprotein or Alpha Fetoglobulin or AFP), Cancer/Testis Antigen 1 (Autoimmunogenic Cancer/Testis Antigen or Cancer/Testis Antigen 6.1 or L Antigen Family Member 2 or CTAG1A or CTAG1B); HBV antigen, EGFR Family Member, Herin, Tyrosine Protein Kinase BTK (Bruton Tyrosine Kinase or B Cell Progenitor Kinase or Agammaglobulinemia Tyrosine Kinase or BTK or EC 2.7.10.2) Inhibitor, CD4, Epithelial Cell Adhesion Molecule (Adenocarcinoma Associated Antigen or Cell Surface Glycoprotein Trop 1 or Epithelial Cell Surface Antigen or Epithelial Glycoprotein 314 or KS 1/4 Antigen or KSA or Tumor Associated Calcium Signal Transducer 1 or CD326 or EPCAM), Prolyl Endopeptidase FAP (170 kDa Melanoma Membrane Bound Gelatinase or Dipeptidyl Peptidase FAP or Integral Membrane Serine Protease or Fibroblast Activation Protein Alpha or Gelatine Degradation Protease FAP or Seprase or FAP or EC 3.4.21.26 or EC 3.4.14.5), Neural Cell Adhesion Molecule 1 (Antigen Recognized By Monoclonal Antibody 5.1H11 or CD56 or NCAM1); Epidermal Growth Factor Receptor (Proto Oncogene c ErbB 1 or Receptor Tyrosine Protein Kinase erbB 1 or HER1 or ERBB1 or EGFR or EC 2.7.10.1) Antagonist, Tyrosine Protein Kinase Transmembrane Receptor ROR1 (Neurotrophic Tyrosine Kinase Receptor Related 1 or ROR1 or EC 2.7.10.1); Wilms Tumor Protein (WT33 or WT1); Interleukin 13 Receptor Subunit Alpha 2 (Interleukin 13 Binding Protein or CD213a2 or IL13RA2), Trophoblast Glycoprotein (M6P1 or 5T4 Oncofetal Antigen or 5T4 Oncofetal Trophoblast Glycoprotein or Wnt Activated Inhibitory Factor 1 or TPBG), SLAM Family Member 7 (CD319 or Membrane Protein FOAP 12 or CD2 Like Receptor Activating Cytocytotoxic Cells or Novel Ly9 or Protein 19A or CD2 Subset 1 or CS1 or SLAMF7), B Cell Lymphoma 2 (Bcl 2) Inhibitor; DNA (Cytosine 5) Methyltransferase 1 (CXXC Type Zinc Finger Protein 9 or DNA Methyltransferase Hsal or MCMT or DNMT1 or EC 2.1.1.37) Inhibitor, ROR1, CD19&CD40L, avidin (EGFRiiiv), a folate receptor, CD30, pmel CD*8 T, CD33, NKR2, Epithelial tumor antigen (ETA), Tyrosinase, Melanoma-associated antigen, abnormal products of ras, p53, Alphafetoprotein (AFP), CA-125, CA15-3, CA27-29, CA19-9, Calcitonin, Calretinin, CD34, CD99MIC 2, CD117, Chromogranin, Cytokeratin (various types: TPA, TPS, Cyfra21-1), Desmin, Epithelial membrane antigen (EMA), Factor VIII, CD31 FL1, Glial fibrillary acidic protein (GFAP), Gross cystic disease fluid protein (GCDFP-15), HMB-45, Human chorionic gonadotropin (hCG), immunoglobulin, inhibin, keratin (various types), lymphocyte marker (various types), BCR-ABL, Myo D1, muscle-specific actin (MSA), neurofilament, neuron-specific enolase (NSE), placental alkaline phosphatase (PLAP), prostate-specific antigen (PSA), PTPRC (CD45), S100 protein, smooth muscle actin (SMA), synaptophysin, thymidine kinase, thyroglobulin (Tg), thyroid transcription factor-1 (TTF-1), Tumor M2-PK, vimentin, SV40, Adenovirus Elb-58kd, IGF2B3, ubiquitous (low level), Kallikrein 4, KIF20A, Lengsin, Meloe, MUC5AC, Immature laminin receptor, TAG-72, HPV E6, HPV E7, BING-4, Calcium-activated chloride channel 2, Cyclin-B1, 9D7, Ep-CAM, EphA3, Telomerase, SAP-1, BAGE family, CAGE family, GAGE family, MAGE family, SAGE family, XAGE family, LAGE-1, PRAME, SSX-2, pme117, Tyrosinase, TRP-1/-2, P.polypeptide, MC1R, f3-catenin, Prostate-pecific antigen, BRCA1, BRCA2, CDK4, CML66, Fibronectin, MART-2, Ras, TGF-beta receptor II, T cell receptor (TCR), BLOC1S6, CD10/Neprilysin, CD24, CD248, CD5/Cluster of Differentiation 5, CD63/Tspan-30/Tetraspanin-30, CEACAM5/CD66e, CT45A3, CTAG1A, CXORF61, DSE, GPA33, HPSE, KLK3, LCP1, LRIG3, LRRC15, megakaryocyte potentiating factor, MOK, MUC4, NDNL2, OCIAD1, PMPCB, PTOV1, RCAS1/EBAG9, RNF43, ROPN1, RPLP1, SARNP, SBEM/MUCL1, TRP1/TYRP1, CA19-9, Inactive Tyrosine Protein Kinase Transmembrane Receptor ROR1 (Neurotrophic Tyrosine Kinase Receptor Related 1 or ROR1 or EC 2.7.10.1), ALK Tyrosine Kinase Receptor (Anaplastic Lymphoma Kinase or CD246 or ALK or EC 2.7.10.1), Prostate Stem Cell Antigen (PSCA), Melanoma Antigen Preferentially Expressed In Tumors (Cancer/Testis Antigen 130 or Opa Interacting Protein 4 or OIP4 or Preferentially Expressed Antigen Of Melanoma or PRAME), Signal Transducer And Activator Of Transcription 3 (Acute Phase Response Factor or DNA Binding Protein APRF or STAT3) Inhibitor, CD44 Antigen (CDw44 or Epican or Extracellular Matrix Receptor III or GP90 Lymphocyte Homing/Adhesion Receptor or HUTCH I or Heparan Sulfate Proteoglycan or Hermes Antigen or Hyaluronate Receptor or Phagocytic Glycoprotein 1 or CD44), CD40 Ligand (T Cell Antigen Gp39 or TNF Related Activation Protein or Tumor Necrosis Factor Ligand Superfamily Member 5 or CD154 or CD40LG) Activator; Tumor Necrosis Factor Receptor Superfamily Member 13B (Transmembrane Activator And CAML Interactor or CD267 or TACI or TNFRSF13B); Cytocytotoxic To Cells Expressing Tumor Necrosis Factor Receptor Superfamily Member 17 (B Cell Maturation Antigen or CD269 or TNFRSF17), CD276 Antigen (B7 Homolog 3 or 4Ig B7 H3 or Costimulatory Molecule or CD276), Myeloid Cell Surface Antigen CD33 (Sialic Acid Binding Ig Like Lectin 3 or gp67 or CD33), ADP Ribosyl Cyclase/Cyclic ADP Ribose Hydrolase 1 (Cyclic ADP Ribose Hydrolase 1 or T10 or 2′ Phospho ADP Ribosyl Cyclase/2′ Phospho Cyclic ADP Ribose Transferase or ADP Ribosyl Cyclase 1 or CD38 or EC 3.2.2.6 or EC 2.4.99.20), C Type Lectin Domain Family 14 Member A (Epidermal Growth Factor Receptor 5 or EGFRS or CLEC14A), Hepatocyte Growth Factor Receptor (Proto Oncogene c Met or Tyrosine Protein Kinase Met or HGF/SF Receptor or Scatter Factor Receptor or MET or EC 2.7.10.1), Epithelial Cell Adhesion Molecule (Adenocarcinoma Associated Antigen or Cell Surface Glycoprotein Trop 1 or Epithelial Cell Surface Antigen or Epithelial Glycoprotein 314 or KS 1/4 Antigen or KSA or Tumor Associated Calcium Signal Transducer 1 or CD326 or EPCAM), Ganglioside GD3, Interleukin 13 Receptor Subunit Alpha 2 (Interleukin 13 Binding Protein or CD213a2 or IL13RA2); Kappa Myeloma Antigen (KMA), Lambda Myeloma Antigen (LMA), Latent Membrane Protein 1 (Protein p63 or LMP1), Melanoma Associated Antigen, Cytocytotoxic To Cells Expressing T Lymphocyte Activation Antigen CD80 (Activation B7-1 Antigen or CTLA 4 Counter Receptor B7.1 or CD80); Cytocytotoxic To Cells Expressing T Lymphocyte Activation Antigen CD86 (Activation B7-2 Antigen or CTLA 4 Counter Receptor B7.2 or CD86), Inactive Tyrosine Protein Kinase Transmembrane Receptor ROR1 (Neurotrophic Tyrosine Kinase Receptor Related 1 or ROR1 or EC 2.7.10.1), Fas Apoptotic Inhibitory Molecule 3 (IgM Fc Fragment Receptor or Regulator Of Fas Induced Apoptosis Toso or TOSO or FAIM3 or FCMR), T Cell Receptor Beta 1 Chain C Region (TRBC1), Vascular Endothelial Growth Factor Receptor 2 (Fetal Liver Kinase 1 or Kinase Insert Domain Receptor or Protein Tyrosine Kinase Receptor flk 1 or VEGFR2 or CD309 or KDR or EC 2.7.10.1), Alpha Fetoprotein (Alpha 1 Fetoprotein or Alpha Fetoglobulin or AFP), Cancer/Testis Antigen 1 (Autoimmunogenic Cancer/Testis Antigen NY ESO 1 or Cancer/Testis Antigen 6.1 or L Antigen Family Member 2 or CTAG1A or CTAG1B), T Cell Surface Glycoprotein CD5 (Lymphocyte Antigen T1/Leu 1 or CD5), Prolyl Endopeptidase FAP (170 kDa Melanoma Membrane Bound Gelatinase or Dipeptidyl Peptidase FAP or Integral Membrane Serine Protease or Fibroblast Activation Protein Alpha or Gelatine Degradation Protease FAP or Seprase or FAP or EC 3.4.21.26 or EC 3.4.14.5), Neural Cell Adhesion Molecule 1 (Antigen Recognized By Monoclonal Antibody 5.1H11 or CD56 or NCAM1), C Type Lectin Domain Family 12 Member A (Myeloid Inhibitory C Type Lectin Like Receptor or Dendritic Cell Associated Lectin 2 or C Type Lectin Like Molecule 1 or CLEC12A), Integrin Alpha V (Vitronectin Receptor Subunit Alpha or CD51 or ITGAV); Cytocytotoxic To Cells Expressing Integrin Beta 6 (ITGB6), Interleukin 13 Receptor Subunit Alpha 2 (Interleukin 13 Binding Protein or CD213a2 or IL13RA2), Trophoblast Glycoprotein (M6P1 or 5T4 Oncofetal Antigen or 5T4 Oncofetal Trophoblast Glycoprotein or Wnt Activated Inhibitory Factor 1 or TPBG), Trophoblast Glycoprotein (M6P1 or 5T4 Oncofetal Antigen or 5T4 Oncofetal Trophoblast Glycoprotein or Wnt Activated Inhibitory Factor 1 or TPBG), C Type Lectin Domain Family 12 Member A (Myeloid Inhibitory C Type Lectin Like Receptor or Dendritic Cell Associated Lectin 2 or C Type Lectin Like Molecule 1 or CLEC12A), SLAM Family Member 7 (CD319 or Membrane Protein FOAP 12 or CD2 Like Receptor Activating Cytocytotoxic Cells or Novel Ly9 or Protein 19A or CD2 Subset 1 or CS1 or SLAMF7), SLAM Family Member 7 (CD319 or Membrane Protein FOAP 12 or CD2 Like Receptor Activating Cytocytotoxic Cells or Novel Ly9 or Protein 19A or CD2 Subset 1 or CS1 or SLAMF7), immunoglobulin, Multidrug resistance-associated protein 3 (MRP3), Proto-oncogene tyrosine-protein kinase ABL1, Prostatic acid phosphatase, OY-TES-1, ACSM2A, Alpha-actinin-4, Perilipin-2, Alpha-fetoprotein, Lymphoid blast crisis oncogene (Lbc) oncoproptein, aldehyde dehydrogenase 1 family member A1 (ALDH1A1), AML, ANKRD17, NY-BR-1, Annexin II, ARHGAP17, ARHGAP30, ARID1B, Endoplasmic reticulum-resident protein, 5′-aminoimidazole-4-carboxamide-1-beta-d-ribonucleotide transfolmylase/inosinicase (AICRT/I), ATR, ATXN2, ATXN2L, BAGE1, BCL11A, Bc1-xL, Breakpoint cluster region, Survivin, Livin/ML-IAP, HM1.24, BTB domain containing 2 (BTBD2), C60RF89, Carbonic anhydrase IX, CLCA2, CRT2, CAMEL, CAN protein, Caspase-5, Caspase-8, KM-HN-1, CCDC88B, cyclin B1, Cyclin D1, CCNI, CDC2, CDC25A, CDC27, CDK12, intestinal carboxylesterase, CEP95, CHAF1A, Coactosin-like 1, CPSF, CRYBA1, TRAG-3, Macrophage colony stimulating factor, CSNK1A1, Melanoma-associated chondroitin sulfate proteoglycan (MCSP), Cathepsin H, Kita-kyushu lung cancer antigen 1, P450 1B1 or CYP1B1, DDR1, DEK oncogene, DEK-CAN, Dickkopf-1 (DKK1), DNAJC8, DSCAML1, EEF2, Elongation factor Tu GTP binding domain containing or SNRP116, EIF4EBP1, Human Mena protein, EP300, ETVS, TEL1 or ETV6, Polycomb group protein enhancer of zeste homolog 2 (EZH2), F2R, F4.2, FAM53C, Fibroblast g, rowth factor 5 or FGFS, Formin-related protein in leukocytes 1 (FMNL1), Fibromodulin (FMOD), FNDC3B, FKHR, GDP-L-fucose, GAS7, GFI1, GIGYF2, GPNMB, 0, A1, GPSM3, GRK2, GRM5, H3F3A, HAUS3, HERC1, HERV-K-MEL, HIVEP2, HMGN, HMHA1, heme oxygenase-1 (HO-1), HNRPL, Heparanase, HMSD-v-encoded mHA, HSPA1A, Hsp70, HSPB1, immediate early response gene X-1 (IEX-1), insulin-like growth factor (IGF)-II mRNA binding protein 3 (IMP-3), IP6K1, IRS2, ITGB8, JUP, RU2AS, KANSL3, KLF10, KLF2, KLK4, KMT2A, K-ras, Low density lipid receptor (LDLR), LDLR-FUT, Mac-2-binding protein, KIAA0205, LPP, LRP1, LRRC41, LSP1, LUZP1, lymphocyte antigen 6 complex locus K (LY6K), MACF1, MAP1A, MAP3K11, MAP7D1, Matrilin-2, Mcl-1, MDM2, Malic enzyme, MEF2D, MEFV, Milk fat globule membrane protein BA46 (lactadherin), Melanotransferrin, GNT-V or N-acetylglucosaminytransferase V, MIIP, MMP14, Matrix metalloproteinase-2, MORC2, Melanoma antigen p15, MUC2, MUM, MYC, MYL9, Unconventional myosin class I gene, N4BP2, NCBP3, NCOA1, NCOR2, NFATC2, NFYC, NIFK, Ninein, NPM, NPM1-ALK1, N-ras, OAS3, P polypeptide, OGT, OS-9, ErbB3-binding protein 1, PAGE-4, P21-activated serine kinase 2 (PAK2), neo-PAP, PARP12, PAX3, PAX3-FKHR, PCBP2, phosphoglycerate kinase 1 (PKG1), PLEKHM2, promyelocytic leukemia or PML, PML-RARA, POLR2A, Cyclophilin B, PPP1CA, PPP1R3B, Peroxiredoxin 5, Proteinase 3, Parathyroid hormone-related protein (PTHrP), Receptor-like protein tyrosine phosphatase kappa, MG50, NY-MEL-1 or RAB38, RAGE, RALGAPB, RAR alpha, RBM, RCSD1, Recoverin, RERE, RGSS, RHAMM/CD168, RPA1, Ribosomal protein L10a, Ribosomal protein S2, RREB1, RSRP1, RTCB, SART, SCAP, Mammaglobin A, Secernin 1, SDCBP, SETD2, SF3B1, Renal ubiquitous protein 1, SIK1, SIRT2, SKI, hairpin-binding protein, SLC35A4, Prostein, SLC46A1, SNRPD1, SOGA1, SON, SOX10, SOX11, SOX2, SOX-4, Sperm protein 17, SPEN, SRRM2, SRSF7, SRSF8, SSX1, SSX2 or HOM-MEL-40, SSX4, STAT1, STEAP, STRAP, ART-1, SVIL, HOM-TES-14/SCP1, CD138, SYNM, SYNPO, SYT, SYT15, SYT-SSX1, SYT-SSX2, SZT2, TAPBP, TBC1D10C, TBC1D9B, hTERT, THNSL2, THOC6, TLK1, TNS3, TOP2A, TOP2B, ATP-dependent interferon-responsive (ADIR), TP53, Triosephosphate isomerase or TPI1, Tropomyosin-4, TPX2, TRG, T-cell receptor gamma alternate reading frame protein (TARP), TRIM68, Prostate-specific protein transient receptor potential-p8 (trp-p8), TSC22D4, TTK protein kinase (TTK), Thymidylate synthase (TYMS), UBE2A, Ubiquitin-conjugating enzyme variant Kua, COA-1, USB1, NA88-A, VPS13D, BING4, WHSC1L1, WHSC2, WNK2, WT1, XBP1, XPO1, ZC3H14, ZNF106, ZNF219, Papillomavirus binding factor (PBF), E3 ubiquitin-protein ligase UBR4.

In some embodiments of the disclosure, the TCR, CAR, and/or SUPRA-CAR may not comprise an antigen-binding domain which binds to an antigen selected from the above recited group of receptors/ligands/targets.

In some preferred embodiments, the TCR, CAR, and/or SUPRA-CAR may comprise an antigen-binding domain which binds to an antigen selected from the group consisting of: CD19, CD20, CD22, GD2, CD133, EGFR, GPC3, CEA, MUC1, Mesothelin, IL-13R, PSMA, ROR1, CAIX, Her2.

Following introduction of the nucleic acid encoding a protein, the NKT cell, T cell, or dendritic cell or NKT cells, T cells, or dendritic cells may be expanded in culture. Suitable methods and reagents for culturing and expanding cells are well-known to the skilled person. Following expansion the methods of the disclosure may further comprise a step of activating the cells with an NKT cell activator, T cell activator, or dendritic cell activator. The NKT cell activator T cell activator, or dendritic cell activator may be as described in detail above.

In some embodiments, AVM NKT, AVM-T cells, and/or AVM-dendritic cells or targeted AVM NKT, AVM-T cells, and/or AVM-dendritic cells are used to deliver a payload such as nucleic acids, dsRNA, siRNA, micro RNA, dsDNA, ssDNA, cDNA, rRNA, mRNA, tRNA, siRNA, dsRNAi, RNAi, organic compounds, cytotoxic drugs, antibodies, vedotin, ozogamicine, emtansine, deruxtecan, mertansine, mafodotin, tubulin inhibitors, Monomethyl auristatin-E (MMAE) and monomethyl auristatin-F (MMAF) are peptide analogs of dolastatin-10, Maytansinoids, vinca alkaloids, calicheamicin, Duocarmycins, pyrrolobenzodiazepine dimers, talirine, tesirine, indolinobenzodiazepine pseudodimers, soravtansine, DM1, DM4, neurotransmitters, DNA intercalators, antimetabolites, endostatins, neurotrophins, chemotherapy, or a growth factor, or an antibody, a toxin, radioactivity, antibiotics, anti-fungal agents, anti-viral agents, receptors, a virus, a cytokine, lipids, a chemokine, peptides and proteins, anti-parasitics, hormones, antigens, neuro-active agents, receptor agonists or antagonists, small molecules, or any type of biologic payload or biologically active payload.

In some embodiments of the disclosure, the cells of the disclosure may be used to deliver a payload that is not one or more of the above recited payloads.

Also provided by the present disclosure are methods of treating cancer, autoimmune disease, or infectious disease (also called microbial disease) in a subject. In some embodiments, the method of treatment is a method of producing a population of natural killer T cells (NKT cells) in a subject as outlined in detail. In some embodiments, the method of treatment is a method of mobilizing a population of NKT cells in a subject as described elsewhere herein. In some embodiments, the method of treatment is a method of producing a population of T cells in a subject as outlined in detail above. In some embodiments, the method of treatment is a method of producing a population of dendritic cells in a subject as outlined in detail above. In some embodiments, the method of treatment is a method of producing a population of NKT cells, T cells, and/or dendritic cells in a subject as outlined in detail above. In other embodiments, the method of treatment is a method comprising administering to a subject a therapeutically effective dose of the isolated NKT cells, T cells, and/or dendritic cells of the disclosure. These may be any of the isolated NKT cell or population of NKT cells, isolated T cell or population of T cells, and isolated dendritic cell or population of dendritic cells outlined above, including the expanded and non-expanded, and/or activated or non-activated and/or transfected or non-transfected cells described above. In these embodiments, the subject, cancer, autoimmune disease, infectious disease, and/or mechanism of therapeutic efficacy may be as described in detail above.

In embodiments in which the method of treatment is a method comprising administering to a subject a therapeutically effective dose of the isolated NKT cells, T cells, and/or dendritic cells of the disclosure, the subject to which the isolated cells are administered may be the same subject from which the cells were isolated. In such embodiments, the treatment may be referred to as an autologous cell treatment. The term “autologous” refers to any material derived from the same individual to which it is later re-introduced, whether the individual is a human or other animal. In other embodiments in which the method of treatment is a method comprising administering to a subject a therapeutically effective dose of the isolated NKT cells, T cells, and/or dendritic cells of the disclosure, the subject to which the isolated cells are administered may be different to the subject from which the cells were isolated. In such embodiments, the treatment may be referred to as an allogeneic cell treatment. The term “allogeneic” refers to any material derived from one individual which is then introduced to another individual of the same species, whether the individual is a human or other animal. That is, in embodiments in which the method of treatment is a method comprising administering to a subject a therapeutically effective dose of the isolated NKT cells, T cells, and/or dendritic cells of the disclosure, the cells can be from either an autologous or allogeneic source.

The methods of treating cancer, autoimmune disease, or infectious disease in a subject according to the present disclosure may further comprise a step of administering an NKT cell activator, T cell activator, and/or dendritic cell activator to the subject. These may be as described in detail above.

As used herein, the term “administering” refers to the physical introduction of an agent to a subject, using any of the various methods and delivery systems known to those skilled in the art. Exemplary routes of administration for the agents disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the agents disclosed herein may be administered via a non-parenteral route, e.g., orally. Other non-parenteral routes include a topical, epidermal, or mucosal route of administration, for example, intranasally, vaginally, rectally, sublingually or topically.

The phrase “systemic injection” as used herein non-exclusively relates to intravenous, intraperitoneally, subcutaneous, via nasal submucosa, lingual, via bronchoscopy, intravenous, intra-arterial, intra-muscular, intro-ocular, intra-striatal, subcutaneous, intradermal, by dermal patch, by skin patch, by patch, into the cerebrospinal fluid, into the portal vein, into the brain, into the lymphatic system, intra-pleural, retro-orbital, intra-dermal, into the spleen, intra-lymphatic, among others.

The term ‘site of injection’ as used herein non-exclusively relates to intra-tumor, or intra-organ such as the kidney or liver or pancreas or heart or lung or brain or spleen or eye, intra-muscular, intro-ocular, intra-striatal, intradermal, by dermal patch, by skin patch, by patch, into the cerebrospinal fluid, into the brain, among others.

In some preferred embodiments of the disclosure, the glucocorticoid-receptor modulating agents may be administered orally. In embodiments in which the method of treatment of the disclosure is a method comprising administering to a subject a therapeutically effective dose of the isolated NKT cells, T cells, and/or dendritic cells of the disclosure, the cells may be applied directly to an organ or tumor via collagen matrices, extracellular matrix compositions, biopolymer microthreads made of fibrin or other extracellular matrix material, patches containing extracellular matrix and biodegradable materials, fibrin patches, alginateor agarose based patches, scaffolds composed of extracellular matrix materials and biodegradable physiologically inert material that could non-exclusively relates to components such as dextrans, coating stem cells with organ specific antigens or binding molecules, remnant extracellular matrices also known as scaffolds or decellularized organs from ex vivo digested organ donors or cadaveric organs, and contact lenses among others. Preferably the cells are administered to the subject by a method selected from the group consisting of: intravenous injection, intraperitoneal injection, intra-lymphatic injection, intrathecal injection, injection into the cerebrospinal fluid (CSF), direct injection into a tumor, or as a gel placed on or near a solid tumor.

In some embodiments of the disclosure, the route of administration for the agents and cells disclosed herein may not be one or more of the above recited routes.

The present disclosure also provides glucocorticoid-receptor (GR) modulating agents and ICAM3 modulating agents for use in a method of producing a population of natural killer T cells (NKT cells), a method of producing a population of T cells, and/or a method of activating a population of dendritic cells as described in detail above. The present disclosure also provides glucocorticoid-receptor (GR) modulating agents and ICAM3 modulating agents, for use in a method of treating cancer, autoimmune disease, or infectious disease (also called microbial disease) in a subject, wherein the method of treatment is a method of producing/activating/mobilizing a population of natural killer T cells (NKT cells) in a subject as described in detail above. Preferred embodiments include glucocorticoids for use in a method of producing a population of natural killer T cells (NKT cells), method of producing a population of T cells, and/or method of activating a population of dendritic cells as described in detail above, and glucocorticoids for use in a method of treating cancer, autoimmune disease, or infectious disease in a subject, wherein the method of treatment is a method of producing a population of natural killer T cells (NKT cells), method of producing a population of T cells, and/or method of activating a population of dendritic cells in a subject as described in detail above. Other preferred embodiments include glucocorticoids for use in a method of mobilizing a population of NKT cells as described in detail above. In some particularly preferred embodiments, the glucocorticoid is dexamethasone.

Also provided by the disclosure is the use of glucocorticoid-receptor (GR) modulating agents or ICAM3 modulating agents in the manufacture of a medicament for use in a method of producing a population of natural killer T cells (NKT cells), method of producing a population of T cells, and/or method of activating a population of dendritic cells as described in detail above. The present disclosure also provides use of glucocorticoid-receptor (GR) modulating agents or ICAM3 modulating agents in the manufacture of a medicament for use in a method of treating cancer, autoimmune disease, or infectious disease (also called microbial disease) in a subject, wherein the method of treatment is a method of producing a population of natural killer T cells (NKT cells), method of producing a population of T cells, and/or method of activating a population of dendritic cells in a subject as described in detail above.

The present disclosure also provides the use of a glucocorticoid-receptor (GR) modulating agent or ICAM3 modulating agent to induce a population of natural killer T cells (NKT cells), wherein the population of natural NKT cells is induced by a method of producing a population of natural killer T cells (NKT cells) in a subject as described in detail above. The present disclosure also provides the use of a glucocorticoid-receptor (GR) modulating agent or ICAM3 modulating agent to induce a population of T cells, wherein the population of T cells is induced by a method of producing a population of T cells in a subject as described in detail above. The present disclosure also provides the use of a glucocorticoid-receptor (GR) modulating agent or ICAM3 modulating agent to activate a population of dendritic cells, wherein the population of dendritic cells is activated by a method of activating a population of dendritic cells in a subject as described in detail above

The present disclosure also provides a method of producing induced pluripotent stem cells (iPSCs), the method comprising reprogramming NKT cells, T cells, or dendritic cells of the disclosure to produce iPSCs. The NKT cells, T cells, or dendritic cells of the disclosure to be used in a method of producing iPSCs, may be NKT cells produced and isolated by a method of producing a population of natural killer T cells (NKT cells), T cells, or dendritic cells in a subject as described in detail above.

In some embodiments of the disclosed method of producing iPSCs, the reprogramming comprises introducing one or more expression cassettes encoding Oct3/4, Klf4, Sox2, and C-myc into the cells of the disclosure. In some embodiments, the reprogramming comprises introducing Oct3/4, KLF4, Sox2, and c-myc encoding mRNA into the cells. In some other embodiments of the disclosed method of producing iPSCs, the reprogramming may further comprise introducing one or more expression cassettes encoding one or more of: Sox1, Sox3, Sox15, Klf1, Klf2, Klf5, L-myc, N-myc, Nanog, and/or LIN28 into the cells. In other embodiments, the reprogramming may further comprise introducing one or more of: Sox1, Sox3, Sox15, Klf1, Klf2, Klf5, L-myc, N-myc, Nanog, and/or LIN28 encoding mRNA into the cells. Suitable methods for introducing expression cassettes or encoding mRNA into a cell are well known to the skilled person—for example by electroporation, cell microinjection, or liposome-based transfection methods. Use of retroviral systems, including lentiviral and adenoviral systems, to reprogram non-pluripotent cells in iPSCs have been described (Stadtfeld et al, 2008, which is hereby incorporated by reference in its entirety). Reprogramming of adult cells to iPSCs can also be accomplished via plasmid without use of virus transfection systems (Okita et al, 2008, which is hereby incorporated by reference in its entirety).

### Oct-3/4

Oct-3/4 (Pou5fl; cDNA available from Bioclone, San Diego Calif.) is one of the family of octamer (“Oct”) transcription factors, and plays a crucial role in maintaining pluripotency. The absence of Oct-3/4 in Oct-3/4+ cells, such as blastomeres and embryonic stem cells, leads to spontaneous trophoblast differentiation, and presence of Oct-3/4 thus gives rise to the pluripotency and differentiation potential of embryonic stem cells. Various other genes in the “Oct” family, including Oct-3/4's close relatives, Oct1 and Oct6, fail to elicit induction, thus demonstrating the exclusiveness of Oct-3/4 to the induction process.

### Klf Family:

Klf4 of the Klf family of genes is a factor for the generation of mouse iPS cells. Klf2 (cDNA available from Bioclone, Inc., San Diego, Calif.) and Klf4 (cDNA available from Bioclone, Inc., San Diego, Calif.) are factors capable of generating iPS cells, and related genes Klf1 (cDNA available from Bioclone, Inc., San Diego, Calif.) and Klf5 (cDNA available from Bioclone, Inc., San Diego, Calif.) did as well, although with reduced efficiency.

### Sox Family

The Sox family of genes is associated with maintaining pluripotency similar to Oct-3/4, although it is associated with multipotent and unipotent stem cells in contrast with Oct-3/4, which is exclusively expressed in pluripotent stem cells (Bowles et al, 2000, which is hereby incorporated by reference in its entirety). While Sox2 (cDNA available from Bioclone, San Diego, Calif.) was the initial gene used for induction, other genes in the Sox family have been found to work as well in the induction process. Sox1 (cDNA available from Bioclone, Inc., San Diego, Calif.) yields iPS cells with a similar efficiency as Sox2, and genes Sox3 (human cDNA available from Bioclone, Inc., San Diego, Calif.), Sox15, and Sox18 also generate iPS cells, although with decreased efficiency.

### Myc Family

The Myc family of genes are proto-oncogenes implicated in cancer. C-myc (cDNA available from Bioclone, Inc., San Diego, Calif.) is a factor implicated in the generation of mouse iPS cells. However, c-myc may be unnecessary for generation of human iPS cells. Usage of the “myc” family of genes in induction of iPS cells is troubling for the eventuality of iPS cells as clinical therapies, as 25% of mice transplanted with c-myc-induced iPS cells developed lethal teratomas. N-myc (cDNA available from Bioclone, Inc., San Diego, Calif.) and L-myc have been identified to induce instead of c-myc with similar efficiency.

### Nanog

In embryonic stem cells, Nanog (cDNA available from Bioclone, Inc., San Diego, Calif.), along with Oct-3/4 and Sox2, is necessary in promoting pluripotency (Chambers et al, 2003, which is hereby incorporated by reference in its entirety).

### LIN28

LIN28 (cDNA available from Bioclone, Inc., San Diego, Calif.) is an mRNA binding protein expressed in embryonic stem cells and embryonic carcinoma cells associated with differentiation and proliferation (Moss & Tang, 2003, which is hereby incorporated by reference in its entirety).

In some embodiments, the disclosed method of producing iPSCs further comprises a step of inducing differentiation of the iPSCs of the disclosure. In some preferred embodiments, the disclosed methods may further comprise inducing differentiation of the iPSCs of the disclosure into NKT cells. Thus, the present disclosure also provides a method of producing a population of NKT cells, the method comprising differentiating iPSCs produced by a method according to the disclosure into an NKT cell lineage. In some embodiments, the disclosed methods may further comprise inducing differentiation of the iPSCs of the disclosure into T cells. Thus, the present disclosure also provides a method of producing a population of T cells, the method comprising differentiating iPSCs produced by a method according to the disclosure into a T cell lineage. In other embodiments, the disclosed methods may further comprise inducing differentiation of the iPSCs of the disclosure into dendritic cells. Thus, the present disclosure also provides a method of producing a population of dendritic cells, the method comprising differentiating iPSCs produced by a method according to the disclosure into a dendritic cell lineage. Such differentiated cells may be employed in the methods of treating cancer, autoimmune disease, or infectious disease (also called microbial disease) in a subject according to the present disclosure.

Also provided by the present disclosure are isolated NKT cells, isolated T cells, and isolated dendritic cells produced or mobilized by any of the methods disclosed herein, as well as isolated populations of NKT cells, T cells, and dendritic cells produced or mobilized by any of the methods disclosed herein. Also provided are NKT cells, T cells, and dendritic cells, and isolated populations of NKT cells, T cells, and dendritic cells characterized by the patterns of surface proteins described in detail elsewhere herein, and use of such cells in the methods of treatment of the disclosure.

### EXAMPLES

The following examples demonstrate that high dose glucocorticoid receptor agonists, in addition to causing near complete lymphodepletion of peripheral blood lymphocytes (without affecting the cell counts of neutrophils, platelets, RBCs and stem cells), can induce production of a novel population of NKT cells and T cells, as well as mobilise a novel population of activated dendritic cells.

These examples also demonstrate that, in addition to presenting with the known properties of NKT cells, the population of NKT cells induced by high dose glucocorticoid receptor agonists have a novel pattern of expression of surface proteins, which allows them to directly engulf cancer cells and exhibit enhanced cytotoxic efficacy against solid cancers.

High doses of glucocorticoid agonists thus represent a promising therapy for use in the treatment of cancer and diseases mediated by immune cells such as lymphocytes.

### Materials and Methods

Acute high dose dexamethasone may also be referred to herein as Dex, AugmenStem™, PlenaStem™ or AVM0703. The novel population of NKT cells induced following administration of acute high dose dexamethasone (AVM0703) may also be referred to herein as AVM-NKT cells. The novel population of T cells induced following administration of acute high dose dexamethasone (AVM0703) may also be referred to herein as AVM-T cells. The novel population of dendritic cells induced following administration of acute high dose dexamethasone (AVM0703) may also be referred to herein as AVM-dendritic cells.

For initial lymphodepletion studies, naïve C57Bl/6 mice were treated with 18 mg/kg HED DP by oral gavage. Male C57BL/6 mice were obtained from Taconic Bioscience (Germantown, N.Y.) and acclimated to laboratory conditions for at least one week. Mice were dosed once orally with 18 mg/kg Dexamethasone Phosphate (DP) or placebo and kept until timepoint. Each dosed timepoint group was accompanied by a placebo group of the same age and condition according to Table 3. Timepoints 24 hours, 48 hours, 72 hours, 5 days, 7 days, 11 days, 13 days were dosed using GLP grade AVM0703 and placebo. Timepoints 6 hours, 21 days, 28 days, 35 days were dosed using GMP grade AVM0703 and placebo. When mice reached study timepoint, they were euthanized as follows. Mice were anesthetized with isoflurane gas. Once anesthetized, blood was drawn via cardiac puncture and place immediately in heparin-lined microtubes. 10 mL of 5 U/mL of Heparin/PBS was used for infused by slow push for retrograde perfusion via the abdominal aorta to flush out all remaining blood from the vasculature. Subsequently, 250 uL of blood was transferred to a lavender-topped EDTA-lined microtube and transported to by Lynette Brown at Flow Contract Site Labs (Bothell, Wash.) for analysis by flow cytometry. The remaining blood was sent to Phoenix Labs (Mukilteo, Wash.) for Complete Blood Counts and Clinical Chemistries

For characterisation of the induced population of NKT cells (AVM-NKT), naïve C57Bl/6 mice were treated with high dose AVM0703 at 12 to 45 mg/kg HED DP by oral gavage. Peripheral blood was subsequently examined by flow cytometry at pre-determined time intervals to characterize different immune populations. Following treatment with AVM0703, two NKT populations were identified: NKT cells defined as CD3medCD49b+ and a novel AVM-NKT population defined as CD3highCD49b+. AVM-NKT cells can be isolated using fluorescence-activated cell sorting (FACS) for CD3highCD49b+(CD3 high=mean fluorescence intensity above 2×10⁴).

### Example 1—Acute High-Dose of Glucocorticoid Receptor Agonists Results in Near Complete Lymphodepletion of Peripheral Blood Lymphocytes, but Induces a Unique Population of NKT Cells

In international patent application PCT/US2019/054395 the present authors have presented a series of experiments demonstrating that high dose glucocorticoid receptor agonists can cause near complete lymphodepletion of peripheral blood lymphocytes as well as reduce the number of germinal centers in lymphoid organs and deplete thymus lymphocytes. These effects are achieved without substantially affecting cell counts of neutrophils, platelets, RBCs and stem cells (both hematopoietic stem cells, HSCs, and mesenchymal stem cells, MSCs).

Here, studies performed in naïve mice show that administration of high-doses of glucocorticoid receptor agonists results in near complete lymphodepletion of peripheral blood lymphocytes without substantially affecting the cell counts of neutrophils, platelets, red blood cells (RBCs) and stem cells (both HSCs and MSCs). Intriguingly, high-dose glucocorticoid receptor agonists were also found to induce upregulation of NKT cells.

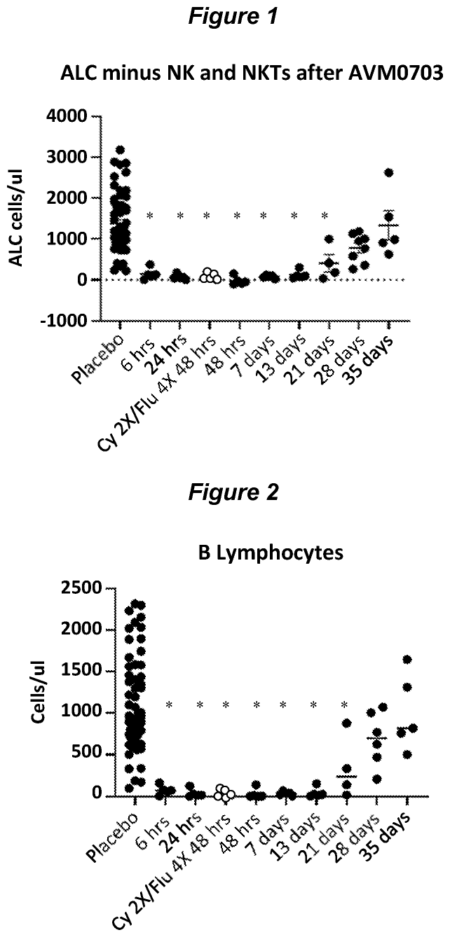

As shown in FIG. 1 high-dose dexamethasone (18 mg/kg HED DP) significantly reduces absolute lymphocyte count (ALC minus NK and NKT cells) as compared to Placebo—an effect that persists for up to 21 days following administration. At 6 and 48 hours after administration almost complete lymphoablation is observed, with the effect comparable to that achieved with standard Cy/Flu chemotherapy (13 mg/kg HED cyclophosphamide and 0.8 mg/kg HED fludarabine).

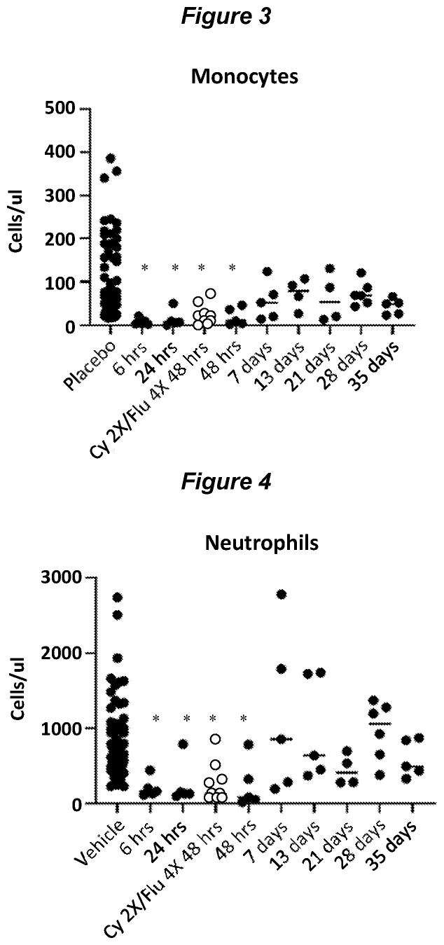

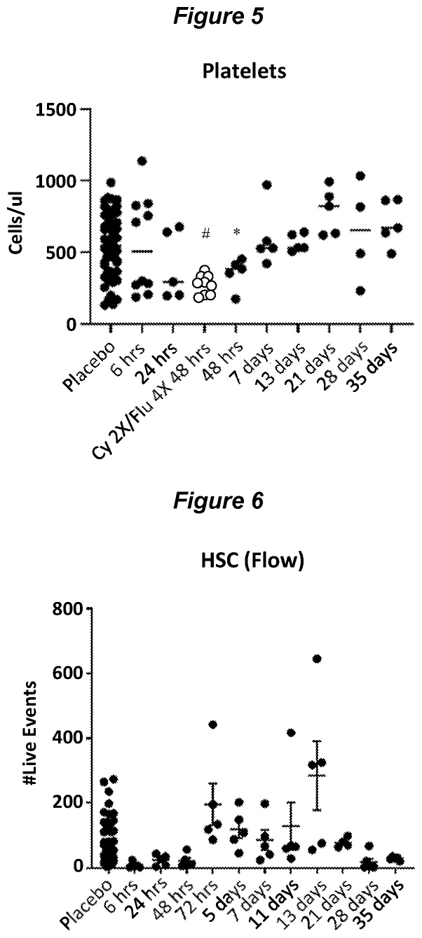

High-dose dexamethasone selectively ablates T and B lymphocytes (equivalently to standard Cy/Flu chemotherapy; FIG. 2), monocytes (superior to Cy/Flu chemotherapy; FIG. 3), and lymphodepletes neutrophils at the target clinical dose (FIG. 4). Basophils (reduced only at the 6 hour time point), eosinophils (reduced only at the 24 and 48 hour time points), platelets (see FIG. 5), and RBCs are all spared, while HSCs (FIG. 6) and MSCs are spared or increased. (* p<0.05; #p<0.0001).

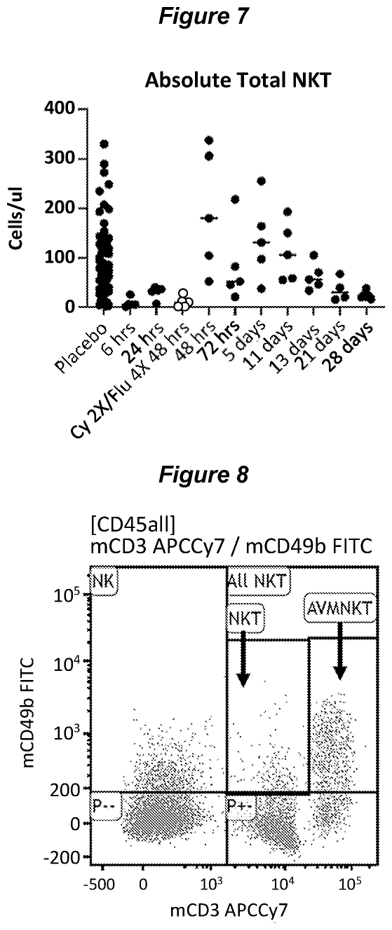

Surprisingly, high-dose dexamethasone was also shown to induce NKT upregulation (FIG. 7) and production of a novel population of NKT cells (AVM-NKT). When examined by flow cytometry these novel AVM-NKT cells are CD49b+ and CD3 very bright (CD3highCD49b+). Previously described NKT cells express CD3 with MFI one log lower than the AVM-NKT cells (CD3medCD49b+; FIG. 8). The AVM-NKT cells appear in the blood of mice 48 hours after administration of high doses (HED 18.1 mg/kg) of the glucocorticoid receptor agonists dexamethasone and betamethasone, but are not induced by standard Cy/Flu chemotherapy.

Dose escalation studies show that a single dose between 6-12 mg/kg HED dexamethasone base can induce AVM-NKT cells. 15 mg/kg HED dexamethasone base induces particularly robust production of the AVM-NKT cells, as does a 6+6 mg/kg HED dosing schedule.

### Example 2—the AVM-NKT Cell is Responsible for In Vivo T and B Lymphoablation

Mononuclear cells from peripheral blood of naïve male C57Bl/6 mice or single cell splenocytes were incubated with equivalent concentrations of AVM0703 as the peak blood concentrations of acute high dose AVM0703 achieve in vivo. Out to 72 hours after addition of AVM0703 to in vitro peripheral blood mononuclear cells or single cell splenocytes, no apoptosis was observed. The lack of in vitro apoptosis of peripheral blood mononuclear cells or splenocytes indicates that the in vivo lymphoablation is due largely to the induction of the AVM-NKT cells.

### Example 3—AVM-NKT Cells Home to Tumor Sites

In preliminary studies, naïve C57Bl/6 mice were treated with high dose dexamethasone with peripheral blood examined by flow cytometry at pre-determined time intervals to characterize the different immune populations. After treatment with high dose dexamethasone, two NKT populations were identified: NKT cells defined as CD3medCD49b+ and the novel population of AVM-NKT defined as CD3highCD49b+(FIG. 8).

AVM-NKT cells were found to appear in the blood of naïve mice 48 hours after supra-pharmacologic doses (HED 18.1 mg/kg) of dexamethasone (AVM0703) or betamethasone. Conversely, these cells are not induced by standard Cy/Flu chemotherapy nor by methylprednisone to any significant extent.

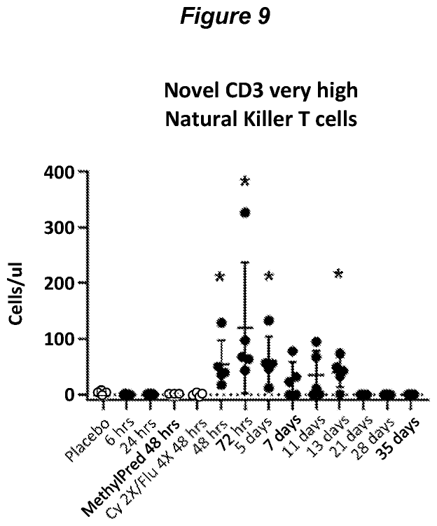

As shown in FIG. 9 and Table 2, in normal mice the AVM-NKT cells are induced in the spleen within 48 hours of dexamethasone dosing, are apparent in peripheral blood from 48 hours after dexamethasone administration, and remain evident in the blood stream until day 13 after dexamethasone administration. AVM-NKT cells are not detected in the spleens of naïve placebo treated mice. Cyclophosphamide/fludarabine dosing does not induce this novel NKT population.

TABLE 2
Presence of AVM-NKT cells in blood, spleen, and tumor in
naïve and A20 mice with and without AVM0703 treatment

| Presence of | 3 hrs | 3 hrs | 3 hrs | 48 hrs | 48 hrs | 48 hrs |
|---|---|---|---|---|---|---|
| AVM-NKT cells | blood | spleen | tumor | blood | spleen | tumor |
| Naïve placebo | ND | ND | NA | negative | negative | NA |
| Naïve AVM0703 | ND | ND | NA | +++ | positive | NA |
| A20 model | + | +++ | + | negative | ND | + |
| placebo | | | | | | |
| A20 model | ++ | −−−− | ++++ | negative | ND | +++ |
| AVM0703 | | | | | | |

NA: not applicable;
ND: not done

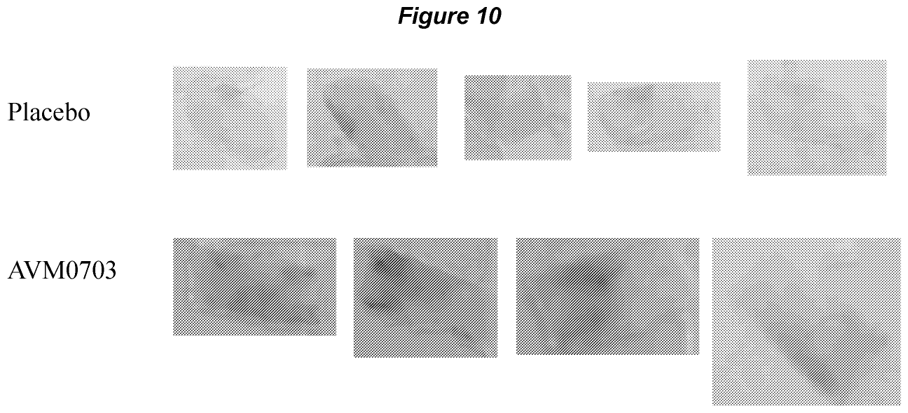

In contrast to the time course of AVM-NKT upregulation observed in normal, disease free mice, quantification of AVM-NKT cells in A20 B cell lymphoma tumor-bearing mice found that AVM-NKT cells are not present in peripheral blood. Instead, in these tumor-bearing mice the AVM-NKT cells appear to home to tumor sites—where increased necrosis is evident when examined 48 hours after dexamethasone administration (FIG. 10).

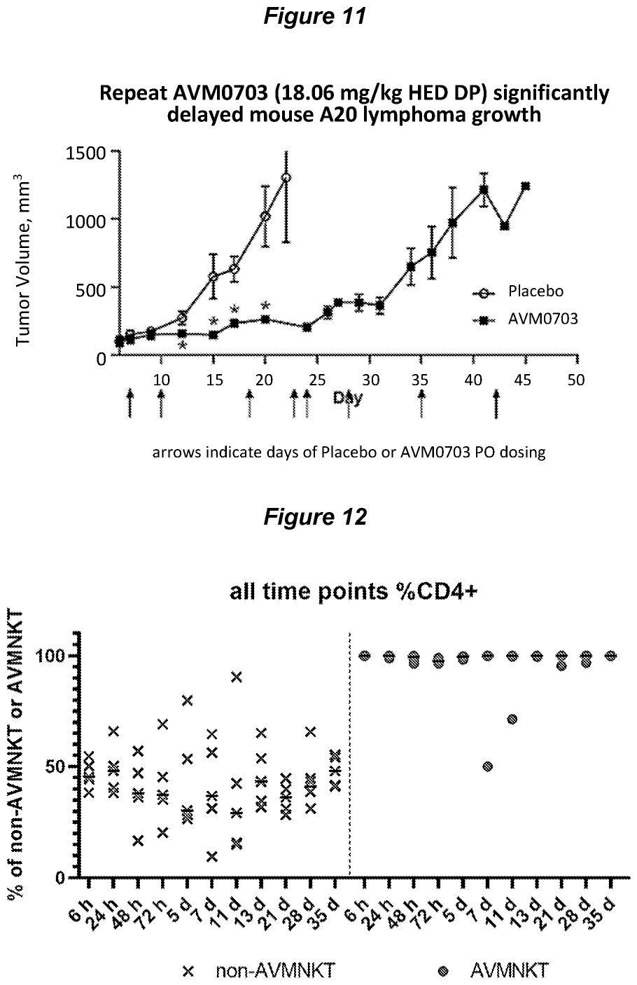

Consistent with this, high dose dexamethasone was shown to significantly delay tumor growth in the A20 model (FIG. 11; Example 15). Because A20 cells undergo only about 30% apoptosis 72 hours after high dose dexamethasone treatment in vitro, it is believed that the AVM-NKT cells play a role in controlling tumor growth.

Two million A20 B lymphoma cells at a cell density of 1.8e7 cells/mL at harvesting were mixed with an equal volume of Matrigel (100 ul each) and injected subcutaneously into the left flank (200 ul total volume) of BALB/c mice, creating a solid tumor model of B cell lymphoma. After tumors were established (approximately 7 days or around −100-150 mm³, which is a well-established tumor), mice were treated according to the dosing table shown below. Tumor volumes were measured with calipers three times a week and the tumor volume was calculated using the equation V=L×W2×0.5. Body weights were also taken three times a week and on days of dosing to determine the proper dosage. Mice were considered to be at study endpoint once they reached a tumor volume of 1500 mm³ or had greater than 20% body weight loss. When mice reached study endpoint, they were euthanized as follows. Mice were anesthetized with isoflurane gas. Once anesthetized, blood was drawn via cardiac puncture and then perfused with 10 mL of 5 U/mL Heparin/PBS. The tumor was removed from the right flank by skinning the right posterior side of the mouse. The skin was stretched out and pinned down, and the tumor was separated from the skin by gently scraping with a scalpel. Tumors were fixed for 48 hours before being transferred to 70% ethanol and stored in cassettes at 4° C. Tumors were shipped to HistotoxLabs (Bolder, Colo.) for sectioning and staining. NKT cells in the tumors were identified by NKp46 staining.

### Example 4—Blood Cancer Enhances the Concentration of AVM-NKT Cells in the Peripheral Blood

Mice are inoculated with T or B cell lymphoma by tail vein injection of 1-5M lymphoma cells in log growth phase. 6 hours to 13 days later blood is harvested from the mice and the AVM-NKT numbers in the blood are determined by flow cytometry gating on CD3 very high (at least 0.5 log higher MFI than T lymphocytes) and CD49b positive cells or by gating on NKp46. Compared to naïve or solid tumor bearing mice, such as T or B lymphoma cells encased in Matrigel and implanted sc in the flank, mice with circulating T or B lymphoma cells have significantly increased numbers of AVM-NKT in the peripheral blood.

### Example 5—AVM-NKT are Induced in Bone Marrow and Fat Tissue 48 Hours after AVM0703 Doses about 29 mg/kg and Higher (Given as DP) in Naïve Balb/c Mice

Balb/c mice have MHC haplotype “d”: H-2K is d (H-2K_{d}). H-2D is d (H-2D_{d}). H2-L is d (H-2L_{D}). Aαβ is d, d. Eαβ is d, d. Mls1 is b. Mls 2 is a. I-A is d (I-A_{d}). I-E is d (I-E_{d}). Qa-1 is b (Qa-1_{b}). Qa-2 is a (Qa-2ₐ).

C57Bl/6 mice have MHC haplotype “b”: H-2K is b (H-2K_{b}). H-2D is b (H-2d_{b}). H2-L is null. Aαβ is b, b. Eαβ is b, b. Mls1 is b. Mls 2 is b. I-A is b (I-A_{b}). I-E is null. Qa-1 is b (Qa-1_{b}). Qa-2 is a (Qa-2ₐ).

The AVM NKT induced in naïve Balb/c mice are CD3 MFI high similar to the peripheral blood AVM-NKT induced in naïve C57Bl/6 mice, and the AVM-NKT in naïve Balb/c mice are TCRgamma/delta positive. Many of the cells are NKp46 negative indicating that they are not activated. This example demonstrates that MEW expression may determine the target organ.

MHC may control the trafficking of AVM NKT cells: The AVM NKT cells are in blood in naïve AVM0703 treated male C57B16 mice. The AVM NKT cells are in fat and bone marrow in naïve AVM0703 treated male Balb/c mice. The AVM NKT cells are in tumors in AVM0703 treated male tumor bearing Balb/c mice. The new NKT in naïve Balb/c mice are also tCRgd positive, B220-, NKp46+/−, Ly6G−, CD4-, CD8-, CD3high, MFI 10492, and CD49b+.

### Example 6—Characterisation of AVM-NKT

Initial studies found that AVM-NKT cells appear in peripheral blood of animals treated with high doses of glucocorticoid receptor agonists (e.g. dexamethasone and betamethasone) around 48 hours after treatment. When examined by flow cytometry the novel population of AVM-NKT cells induced by high-doses of glucocorticoid receptor agonists were found to be CD49b+ and CD3 very bright (CD3highCD49b+). By contrast, previously described NKT cells express CD3 with MFI one log lower than AVM-NKT cells (CD3medCD49b+; FIG. 8).

In subsequent experiments, C57Bl/6 animals were treated with high dose dexamethasone (15 mg/kg HED dexamethasone base) and peripheral blood was examined by flow cytometry at predetermined time intervals to characterize the different immune populations, and in particular the novel population of AVM-NKT cells.

### CD4

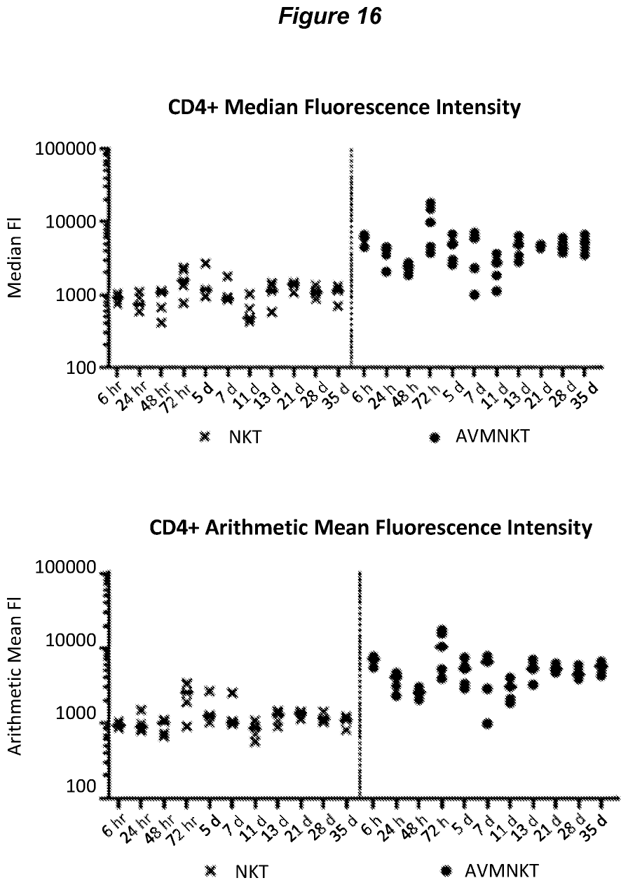

AVM-NKT cells induced by high-doses of glucocorticoid receptor agonists are CD4 very bright (CD4high). CD4 median fluorescence intensity is higher than the CD4 MFI for typical NKT events other CD4+ T cells. CD4 MFI remains constant throughout the 6 hours to 13 days after 15 mg/kg HED dexamethasone base (FIG. 16).

### CD8

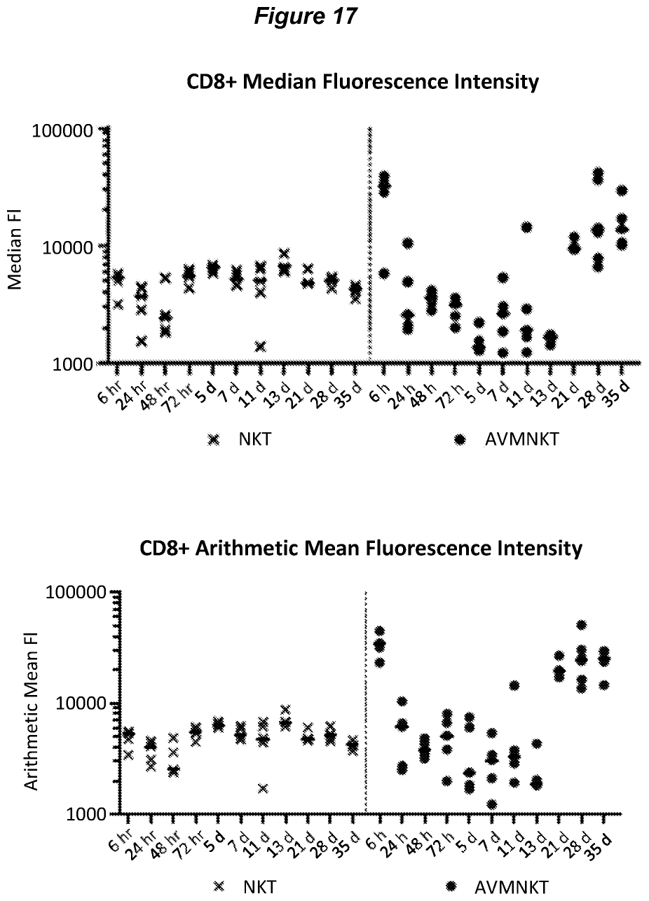

AVM-NKT cells induced by high-doses of glucocorticoid receptor agonists are CD8 dim. CD8+ MFI is not quite 1 log higher than typical NKT cells at the 6 hour time point, and then falls linearly over the next 5 days. Less than 50% remain CD8+ at 72 hours and day 5, but regain CD8+ on days 7-13 after 15 mg/kg HED dexamethasone base (FIG. 17).

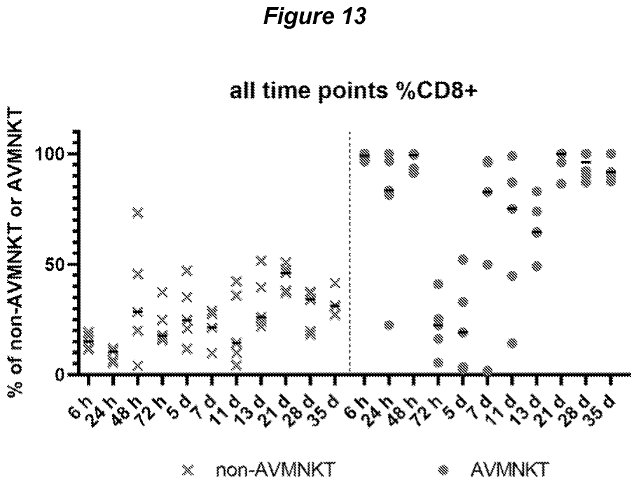

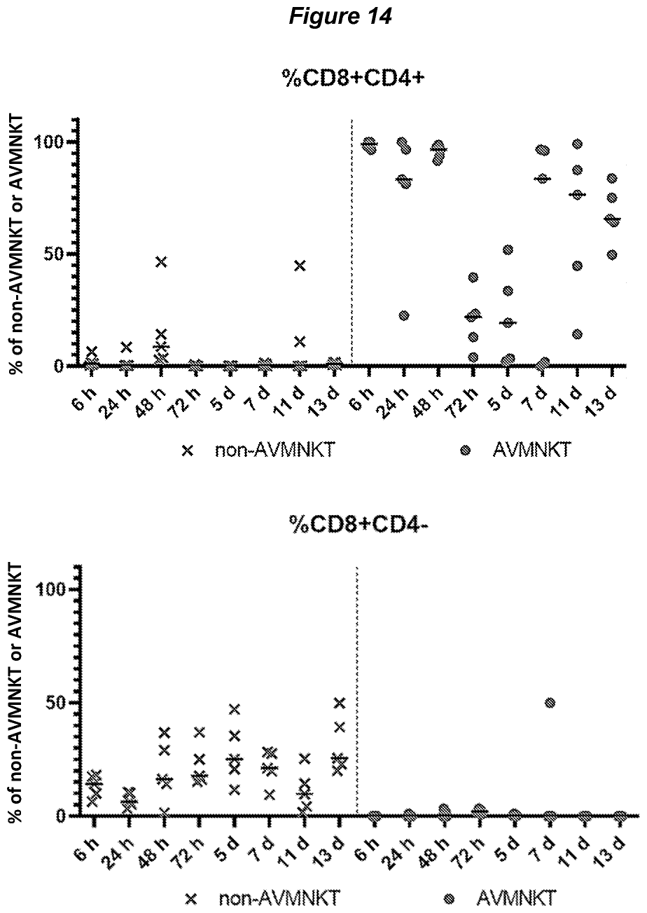

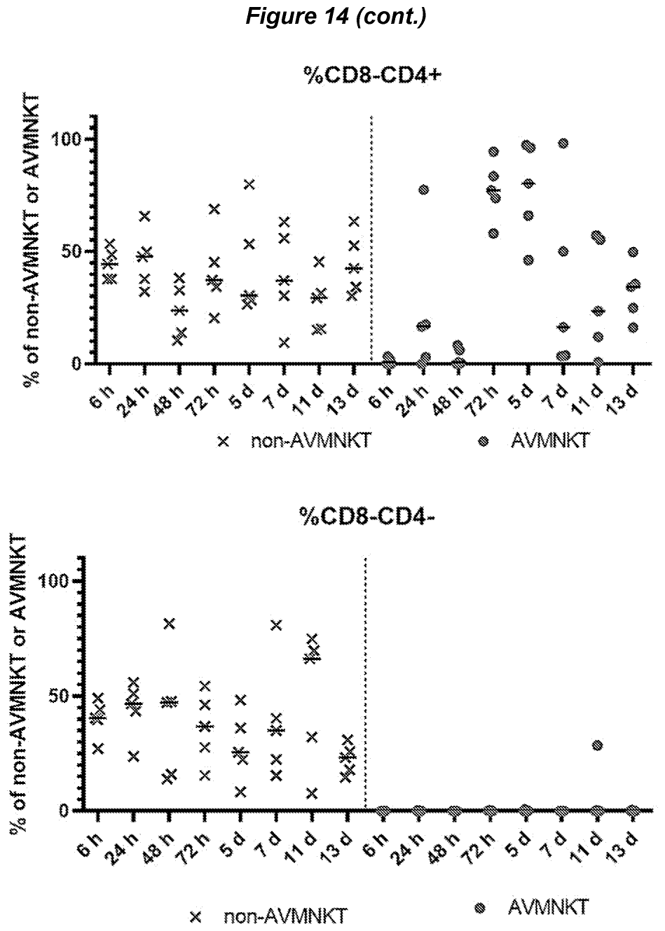

The majority of the AVM-NKT cells are CD4 and CD8 double positive in contrast to typical NKT which are not double positive (FIGS. 12, 13, 14). None of the AVM NKT cells are double negative for CD4 and CD8 (FIG. 14). Known NKT cells (CD3med) are mostly double negative or CD4+, with some CD8+ cells (FIG. 14). For these known NKT cells, the CD4 and CD8 expression pattern does not change with time after dexamethasone base.

AVM-NKT are CD4+CD8+ at 48 hours after 15 mg/kg HED dexamethasone base, lose CD8 positivity over time and then seem to become CD4+CD8+ again at later times points. They are evident at 48 hrs after AVM0703 and found out to day 13.

### CD3

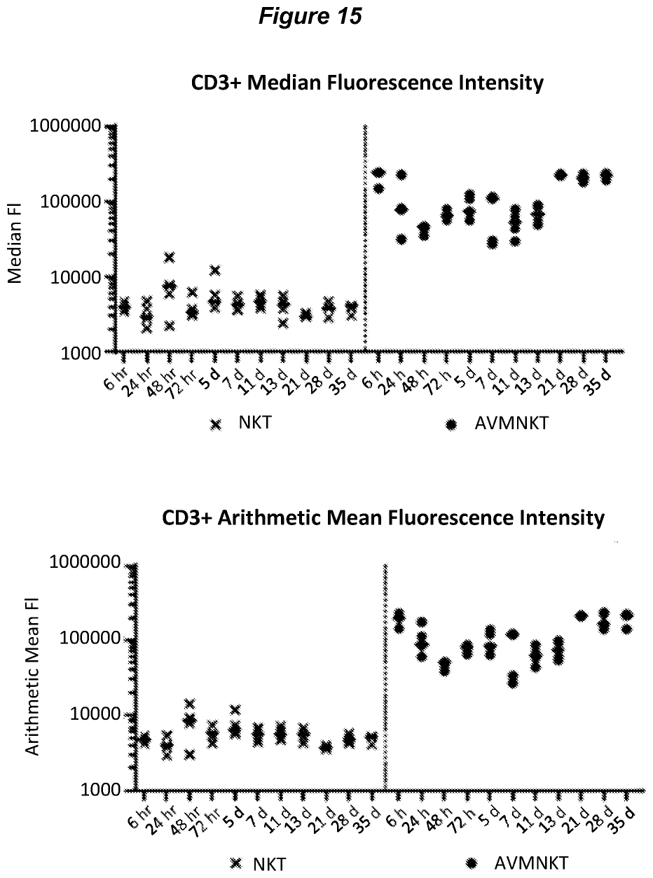

AVM-NKT cells induced by high-doses of glucocorticoid receptor agonists are CD3 very bright (CD3high), expressing CD3 with MFI about one log higher than known NKT cells described in the literature and about 1 log higher than other NKT cells evident in C57Bl/6 male mice (FIG. 15).

### Ly6G

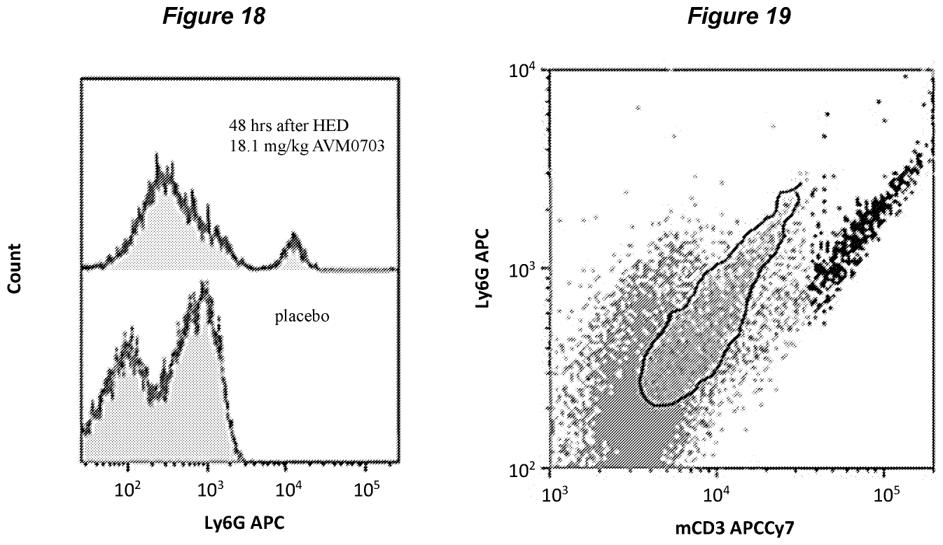

AVM-NKT cells induced by high-doses of glucocorticoid receptor agonists are Ly6G positive (FIG. 18). Ly6G is a marker for fully mature and differentiated neutrophils or granulocytes, and has also been implicated in antitumor responses. Ly6G is usually a marker for monocytes and neutrophils and granulocytes, indicating that AVM-NKT are distinct from known NKT cells, and may not only be able to directly kill cancer cells that express CD1d, as well as activate other NK cells and B and T lymphocytes and secrete cytokines, but may also be able to engulf cancer cells and pathogens directly.

### TCR Gamma/Delta

AVM-NKT cells induced by high-doses of glucocorticoid receptor agonists are TCR gamma delta positive.

The expression of Ly6G and TCR gamma delta suggests that AVM-NKT cells, in addition to having known functions of NKT cells, could also directly engulf cancer cells or pathogens.

### CD45

AVM-NKT cells induced by high-doses of glucocorticoid receptor agonists are CD45 dim.

### CD49b

AVM-NKT cells induced by high-doses of glucocorticoid receptor agonists are CD49b positive. CD49b is a marker of Natural Killer (NK) cells; the cytotoxicity of NK cells expressing CD49b is much greater than NK cells that do not express CD49b.

### CD62L

AVM-NKT cells induced by high-doses of glucocorticoid receptor agonists are CD62L positive.

### NK1.1

In C57Bl/6 mice AVM-NKT cells induced by high-doses of glucocorticoid receptor agonists are NK1.1 positive. NK1.1 is a marker of mature NK cells; its activation induces NK cells to kill otherwise insensitive targets, and may also induce NK cells to proliferate.

### Sca1

AVM-NKT cells induced by high-doses of glucocorticoid receptor agonists are Sca1 very bright. Sca1 (Ly6A) is the common biological marker used to identify hematopoietic stem cell (HSC) along with other markers. Its bright expression on AVM-NKT cells may indicate that these are activated memory stem cells.

### C-Kit

AVM-NKT cells induced by high-doses of glucocorticoid receptor agonists are C-kit negative. Thus they are not hematopoietic stem cells.

### B220

AVM-NKT cells induced by high-doses of glucocorticoid receptor agonists are B220 negative. B220 is a marker for B cells.

### FoxP3

AVM-NKT cells induced by high-doses of glucocorticoid receptor agonists are A FoxP3 negative. FoxP3 is a marker for regulatory cells—thus, AVM-NKT are not regulatory cells and should not dampen the immune response to cancer or a pathogen.

### TCR Alpha/Beta

AVM-NKT cells induced by high-doses of glucocorticoid receptor agonists are TCR alpha/beta negative.

### CD44+/−, CD69+/−, CD25+/−

AVM-NKT cells induced by high-doses of glucocorticoid receptor agonists are CD44+/−, CD69+/−, and CD25+/−.

CD44 expression is an indicative marker for effector-memory T-cells. CD69 and CD25 are markers of cell activation.

Based on the above characterisation experiments, the AVM-NKT cells induced by high dose glucocorticoid receptor agonists appear to be an activated effector memory stem cell type that may have the capacity to rapidly engulf cancer cells and pathogens (they are Ly6G and TCR gamma delta positive), directly kill cancer cells and other cells that present lipid via CD1d expression, and to be able to function as long term T lymphocytes of both the CD4 and CD8 variety. In addition, they may also be able to rapidly release cytokines in response to a cancer cell or pathogen that can activate other cells important for an immune response.

That the AVM-NKT cells appear to add the potential to directly engulf cancer cells, expands the potential for high dose glucocorticoid receptor agonists as therapeutics for solid cancers. AVM-NKT cells as an off-the-shelf allogeneic treatment, either alone or in combination with NKT activators or checkpoint inhibitors or as an AVM-NKT-CAR, could have broad applications in the treatment of solid tumors. Additionally, since the AVM-NKT cells are not observed until after AVM0703 treatment, AVM-NKT numbers may not limit treatment like the low numbers of NKT in elderly and cancer patients limit iNKT use for autologous therapy.

### Example 7—Acute High-Dose Dexamethasone has Tumor Killing Effects in T Cell and B Cell Lymphoma

High dose dexamethasone was shown to significantly delay tumor growth in the A20 B cell lymphoma tumor model (FIG. 11). A subsequent series of dosing experiments was performed in order to investigate the optimal dosing schedule for AVM-NKT production and tumor killing effect in the A20 B cell lymphoma tumor model and a xenograft model of T cell lymphoma (CCRF-CEM). The dosing schedules tested are outlined in Table 3 below.

TABLE 3
Exemplary dosing schedules

AVM0703
Dose(s)

| | | (DP) mg/kg | Dose | Dose | Dose | Dose | Dose | Dose | Dose | Dose | Dose |
|---|---|---|---|---|---|---|---|---|---|---|---|
| Study | Design | HED | 1 | 2 | 3 | 4 | 5 | 6 | 7 | 8 | 9 |
| A20_1 | Repeat | 18 | 7 | 10 | 18 | 23 | 24 | 29 | 36 | 43 | 50 |

| | | | |
|---|---|---|---|
| # End | 1 | 3 | 1 |
| pt after | | | Resistance? |

| 1-10 totally necrotic was dosed as above; | | | | | |
|---|---|---|---|---|---|
| Mouse 1-5 totally necrotic was dosed as shown here | X | X | X | X | X |

| | | | | | | | | |
|---|---|---|---|---|---|---|---|---|
| A20-3 | DRC | 7, 18, 25 | 10 | 17 | 25 | 32 | 39 | 18&26 mg/kg 50-80% necrotic |

| | Saw a necrosis DRC and CD3 labeling inversely related to DRC | | | | | | | | | |
|---|---|---|---|---|---|---|---|---|---|---|
| A20_4 | Combo CyFlu | 18 | 11 | AVM | 14 | AVM | | | | |
| | | | | AVM | | CyFlu | 67 | AVM | 74 | CyFlu |
| | | | | CyFlu | | CyFlu | | | | |

| A20_5 | DRC | 18, 22, 25 | 15 | 31 | 38 | 45 |
|---|---|---|---|---|---|---|
| A20_7 | AVM, APP, Mylan | 18 | 9 | 16 | 23 | |

A20 is a mouse B lymphoma

| | | | | | | | | | | | |
|---|---|---|---|---|---|---|---|---|---|---|---|
| CCRF-CEM | Xeno human | 18 | 7 | 14 | 21 | 28 | 35 | 42 | 49 | 56 | 63 |
| | T lymphoma | | | | | | | | | | |

| | No tumor growth whatsoever |
|---|---|
| DRC: dose response curve | Dose is how many days after |
| | tumor implantation |

### Example 8—AVM-NKT Cells are Responsible for the In Vivo T and B Lymphocyte Ablation

Blood and spleens were taken from naïve C57B16 mice. Mononuclear cells were isolated from blood and single cell splenocytes were isolated from the spleen. The cells were incubated with dexamethasone phosphate up to 500 micromolar concentrations for out to 72 hours, however, no in vitro apoptosis was observed. Therefore, the complete in vivo T and B lymphocyte ablation appears to be mediated by the AVM-NKT, and not a receptor based mechanism such as activation of a glucocorticoid receptor.

### Example 9—AVM-NKT Cells are Isolated and Expanded then Used to Precondition a Patient Before a Cell Therapy

Autologous or allogeneic AVM-NKT cells are administered either IV or IP to a patient between 6 to 96 hours before a cell therapy is administered. The cell therapy can be for a regenerative purpose, for treating a cancer, for treating an autoimmune disease or for treating an infection or any other medical condition that warrants cell therapy.

### Example 10—AVM-NKT Induce Tumor Lysis Syndrome

AVM-NKT target to tumors and form bands of attacking cells invading the tumor like an army from all sides. Tumor lysis syndrome occurs, and in mice, cannot be treated and can cause death. Clinical chemistry markers of tumor lysis syndrome are elevated, such as uric acid. Gross examination of tumors shows a sludge-like oil encased in the tumor membrane.

### Example 11—AVM-NKT Cells are Used to Prepare a Patient for Cancer or Other Serious Medical Treatment

Autologous or allogeneic AVM-NKT cells are administered either IV or IP to a patient with a performance status that prevents them from having a medical therapy such as chemotherapy, cell therapy, organ or bone marrow transplant. The patient's performance status improves such that they become eligible for medical treatment.

### Example 12—AVM-NKT Cells Cause Tumor Pseudoprogression

Tumors treated with AVM-NKT cells continue to appear to grow, however, the growth is pseudoprogression of the tumor because of the other immune cells that the AVM-NKT cell attracts to the tumor, either through the release of cytokines and chemokines or by direct engagement of other immune cells. Eventually, the tumor becomes completely acellular and is resorbed.

### Example 13—AVM-NKT Cells is Used to Treat any Type of Cancer, Graft Versus Host Disease, Autoimmunity, or Immune-Related Adverse Events of Immunotherapies

AVM-NKT cells home to and target both blood and solid cancers, and fibroid tumors, benign tumors, and autoreactive T and B lymphocytes.

### Example 14—Acute High-Dose of Glucocorticoid Receptor Agonists Also Induces a Unique Population of T Cells and Dendritic Cells

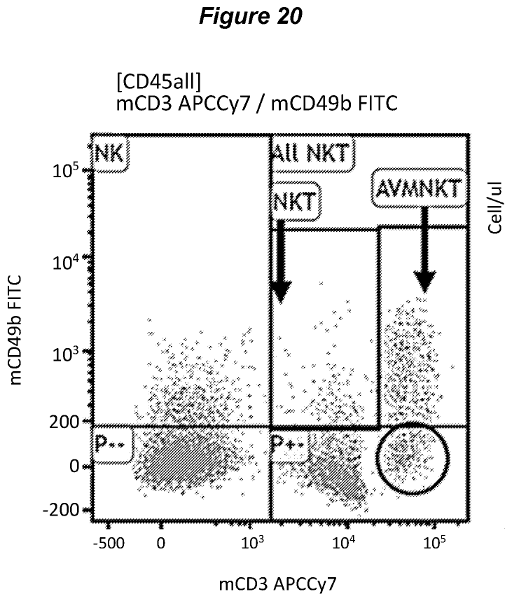

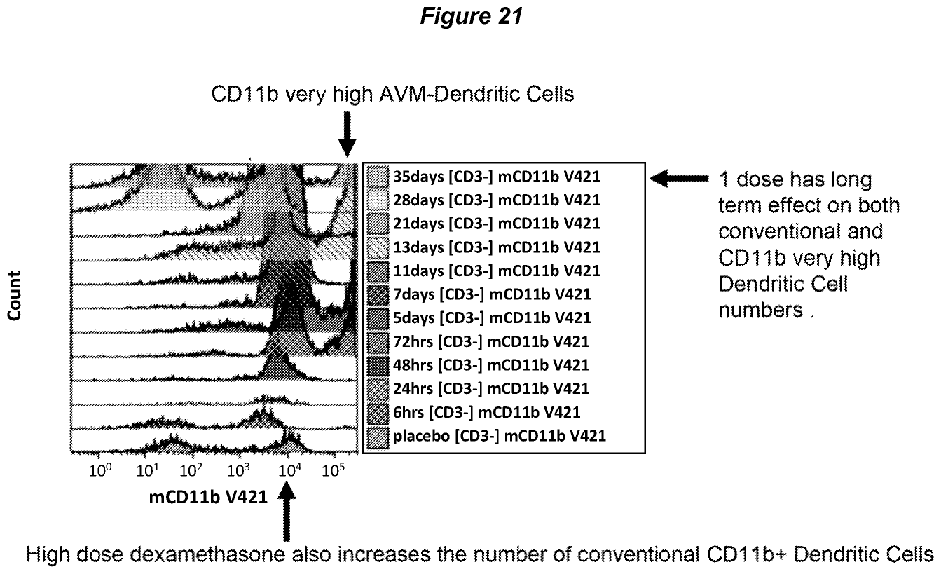

In addition to the novel population of NKT cells (AVM-NKT) described in Examples 1-6, the present authors have shown that high dose dexamethasone mobilises a novel population of CD3 very high T cells (AVM-T cells; FIG. 20), and of CD11b very high dendritic cells (AVM-dendritic cells; FIG. 21).

AVM-T cells induced by high-doses of glucocorticoid receptor agonists are CD3 very bright (CD3high). Like the novel AVM-NKT cells, the novel AVM-T cells express CD3 at 1-1.5 logs higher than typical T or NKT cells (FIG. 20). AVM-T cells induced by high-doses of glucocorticoid receptor agonists are CD4 positive. AVM-T cells induced by high-doses of glucocorticoid receptor agonists are CD45 dim. AVM-T cells induced by high-doses of glucocorticoid receptor agonists are CD49b positive (CD56 positive in humans). AVM-T cells induced by high-doses of glucocorticoid receptor agonists are CD8 positive.

AVM-dendritic cells induced by high-doses of glucocorticoid receptor agonists are CD11b very bright (CD11b very high). The CD11b very high AVM-dendritic cells express CD11b about 1 log higher than conventional CD11b+ dendritic cells. High dose dexamethasone also increases the concentration of conventional CD11b+ dendritic cells in the blood (FIG. 21).

Because high dose glucocorticoid (dexamethasone) mobilizes a novel CD3 very high Natural Killer T cell (AVM-NKT), a novel CD3 very high T cell (AVM-T cell), and a CD11b very high dendritic cell (AVM-dendritic cell), it is believed that high dose glucocorticoids, such as dexamethasone and betamethasone, will have clinical utility in treatment of cancers, autoimmune diseases, and infectious diseases.

The data presented in Examples 14 and 15 demonstrates that the novel AVM-NKT homes remarkably to tumors for tumor killing, and is effective in cancer models in which checkpoint inhibitors have been shown to be ineffective. Because AVM-NKT cells are mobilized only after AVM0703 treatment (as opposed to other NK and NKT which circulate continuously), AVM-NKT numbers may not be limiting in patients.

iNKT cells have been shown to reduce Influenza A mediated inflammation and disease severity, and CD11b+DC have been implicated in protection against Respiratory Syncytial Virus and Influenza A (H1N1). Because cells with lower CD3 and CD11b expression levels are known to be effective in these, it is likely that high dose glucocorticoids, such as dexamethasone and betamethasone, should be even more effective in view of the CD3 very high NKT and T cells mobilized, and because it not only increases the number of conventional CD11b+ dendritic cells, it also mobilizes a CD11b very high expressing dendritic cell that is not typically observed.

### Example 15—Acute High-Dose Dexamethasone Reduces Tumor Volume and Improves Overall Survival in the A20 Model of B Cell Lymphoma

The mouse A20 lymphoma model is a very aggressive tumor model because it employs multiple direct (expression of immunoinhibitory molecule PD-L1, IDO, and IL-10, and lack of expression of CD80 costimulatory molecule) and indirect (downregulation of APC function and induction of Treg cells) immune evasion mechanisms. In addition, in the studies described in the following, AVM0703 was not dosed until A20 tumors were very well-established, at between ˜120 to 400 mm³ in volume.

Male BALB/c mice were inoculated subcutaneously in the flank with A20 B lymphoma cells embedded in Matrigel. Tumor volumes were monitored by caliper measurements, and when the tumors were well established at about 150 mm³, or very well established for study “AVM_CANMOD_05” at about 400 mm³, mice were treated with AVM0703 at HED doses of 7, 18, 22, or 25 mg/kg. Endpoint is typically defined as tumor volume of 1500 mm³.

Analysis of tumors scored blindly for necrosis by HistoTox Labs (Boulder, Colo.) or for viable tumor using MetaMorph analysis or microscopic brightfield assessment demonstrated that some AVM0703 treated mice had completely necrotic and even completely resorbed tumors despite measurable tumor volumes. Necrosis, scored blindly by HistoTox Labs, was significantly higher when the 18, 22, and 25 mg/kg dose results were combined, and was also significantly higher when the 22 and 25 mg/kg AVM0703 treated mice were analyzed separately.

Some AVM0703 treated mice had no measurable tumor at study end, or the tumors were completely necrotic or resorbed by MetaMorph or microscopic brightfield examination, or the tumors received a maximal necrosis score of 5 by HistoTox Labs. These mice were pooled and a contingency table analysis was performed using Fisher's exact test. Of 52 mice treated with AVM0703 between 18 and 25 mg/kg, 10 mice had a complete response based on the preceding criteria; compared to 0 of the 21 placebo-treated mice.

Taken together, these data indicate that AVM0703 has substantial efficacy against aggressive lymphoma at HED of 18 mg/kg and greater (Tables 4 & 5). AVM0703 treatment has also found to exert profound inhibition on a human T cell lymphoma CCRF-CEM growth in a pilot xenograft model (see Example 15).

TABLE 4
Summary of A20 B-Cell Lymphoma Models

| | | | | | MetaMorph | Completely |
|---|---|---|---|---|---|---|
| | | | HistoTox Labs | MetaMorph | Area of Live | Necrotic/ |
| | | | Necrosis H&E | Percent | Tumor | Resorbed By |
| | | | (0-5) | Necrotic | (AU × 1000) | Any Measure |
| Placebo | | Average | 2.3 n = 15 | 50 n = 17 | 90 n = 17 | 0 of 21 |
| AVM0703 | 18, 22, and | Average | 2.9* n = 41 | 59 n = 40 | 75 n = 40 | **10 of 52 |
| | 25 mg/kg | | | | | |
| | 18 mg/kg | Average | 2.7 n = 15 | 59 n = 18 | 66\hat{ } n = 18 | **8 of 26 |
| | 22 mg/kg | Average | 3.0* n = 11 | 53 n = 11 | 87 n = 11 | 0 of 11 |
| | 25 mg/kg | Average | 3.1* n = 13 | 63 n = 11 | 77 n = 11 | 2 of 13 |

*p < 0.05 unpaired t-test Welch's correction.
\hat{ }p < 0.06 unpaired t-test Welch's correction.
**p < 0.05 Fisher's exact test.
AU = arbitrary units; H&E = hematoxylin and eosin.

TABLE 5
Individual Study Results for Necrosis, Percent Dead Area,
Live Area, and Microscopic Necrosis/Percent Acellular

| | | | Days | | | | |
|---|---|---|---|---|---|---|---|
| | | | From | | | | EVOS |
| | | | Last | HistoTox | | MetaMorph | Brightfield |
| Tumor | | | Dose to | Labs | MetaMorph | Area of Live | Percent |
| mm³ at | | Day(s) | Tumor | Necrosis | Percent | Tumor | Necrotic/ |
| Dosing | Group | of Dose | Harvest | H&E (0-5) | Necrotic | (AU × 1000) | Resorbed |
| 391 mm³ [1] | Placebo | 16 | 2 | 3, 2, 2, | 87, 57, | 36, 91, 228, | ND |
| | | | | 2.5, 3 | 52, 39, 81 | 141, 54 | |
| | 18 mg/kg | | | 4, 4, | 84, 91, | 50, 55, | ND |
| | | | | 3, 3, 2 | 78, 68, 78 | 48, 74, 19 | |
| | 22 mg/kg | | | 3, 3, 3, 4 | 82, 64, 72, 83 | 75, 95, 52, 81 | ND |
| | 25 mg/kg | | | 5, 5, 3, 2 | 100, 72, 78, 51 | 6, 54, 157, 48 | ND |
| 120 mm³ [2] | Placebo | 7, 10, | | ND | ND | ND | 10, 20 |
| | 18 mg/kg | 18, 23, | 3, 3, 6, | | | | 100, 100, |
| | | 24, 29, | 6, 3, 3 | | | | 100, 100, |
| | | 36, 43 | | | | | 100, 100 |
| 158 mm³ [3] | Placebo | 9, 16, | | 2, 2 | | | ND |
| | 7 mg/kg | 23, 32 | 3 | 3, 3 | | | |
| | 18 mg/kg | | 3 | 5, 4 | | | |
| | 25 mg/kg | | 3 | 3, 4 | | | |
| 142 mm³ [4] | Placebo | 11, 14 | 14-28 | ND | 60, 54, 64, 29 | 87, 97, 51, 182 | ND |
| | 18 mg/kg | 11, 14 | 18-28 | | 43, 53, 68, 50 | 108, 107, 50, 112 | |
| | Cy/Flu | 11, 14 | 76-81 | | 36, 100, 100 | 169, 0, 0 | |
| | 18 mg/kg | 11, 14 | 6, 66-81 | | 90, 100, 91, | 11, 0, 35, | |
| | Cy/Flu | | | | 65, 61 | 25, 11 | |
| 384 mm³ [5] | Placebo | 15, 31, | | 2.5, 2, 3.5, 2, 3, | 75, 38, 28, 47, | 26, 87, 110, 49, | ND |
| | | 38, 45 | | 2.5, 1.5, 1.5 | 59, 15, 31, 43 | 80, 80, 71, 73 | |
| | 18 mg/kg | | 4-13 | 2, 1, 2, 2.5, | 71, 17, 36, 100, | 53, 95, 76, 0, | Mouse 11 |
| | | | | 3, 3, 2.5, 1 | 51, 33, 35, 42, | 67, 90, 97, 68, | tumor |
| | | | | | 69 | 27 | completely |
| | | | | | | | resorbed |
| | 22 mg/kg | | 1-15 | 2.5, 3, 3, 3, | 26, 43, 62, 36, | 77, 67, 56, 87, | ND |
| | | | | 2, 3.5, 2.5 | 29, 43, 45 | 76, 136, 152 | |
| | 25 mg/kg | | 1-13 | 2, 2.5, 2, 2.5, | 56, 54, 55, 54, | 84, 124, 131, | ND |
| | | | | 4, 3, 2.5 | 67, 37, 69 | 78, 86, 38, 44 | |

[1] Study AVM_CANMOD_05 - lymphodepletion subset.
[2] Study AVM_CANMOD_01.
[3] Study AVM_CANMOD_03.
[4] Study AVM_CANMOD_04.
[5] Study AVM_CANMOD_05 - endpoint analysis subset.
AU = arbitrary units; Cy/Flu = cyclophosphamide/fludarabine; H&E = hematoxylin and eosin; ND = not determined.

### AVM_CANMOD_01

In a first study (“AVM_CANMOD_01”), the ability of AVM0703 to reduce tumor volume and its impact on overall survival was investigated in BALB/c mice (11 weeks old) with well-established subcutaneous A20 tumors. Mice were randomized into 2 groups and treated orally with 18.06 mg/kg AVM0703 HED DP (n=5) or placebo (n=4) on Days 7, 10, 18, 23, 24, 29, 36, and 43 post inoculation.

Of the 5 AVM0703 treated mice, 4 mice reached endpoint after 7 doses each and 1 mouse reached endpoint after 8 doses. Study endpoint was defined as either a tumor volume of 1500 mm³ or greater than 20% body weight loss. With 8 doses each at 18.06 mg/kg, the total dose the latter mouse received was 145 mg/kg HED within 36 days. Mice were euthanized if they reached the endpoint and organs (colon, spleen, pancreas, and thymus) were examined during necropsy and weighed.

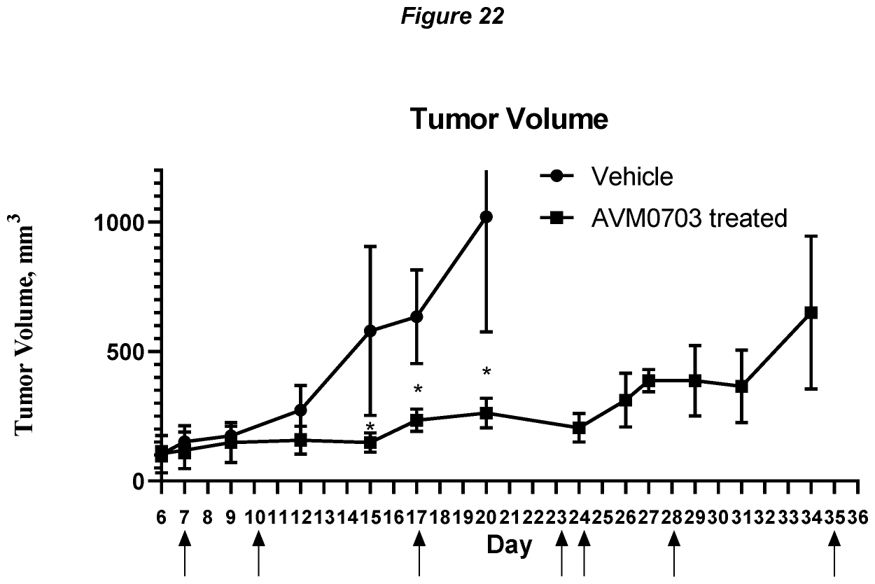

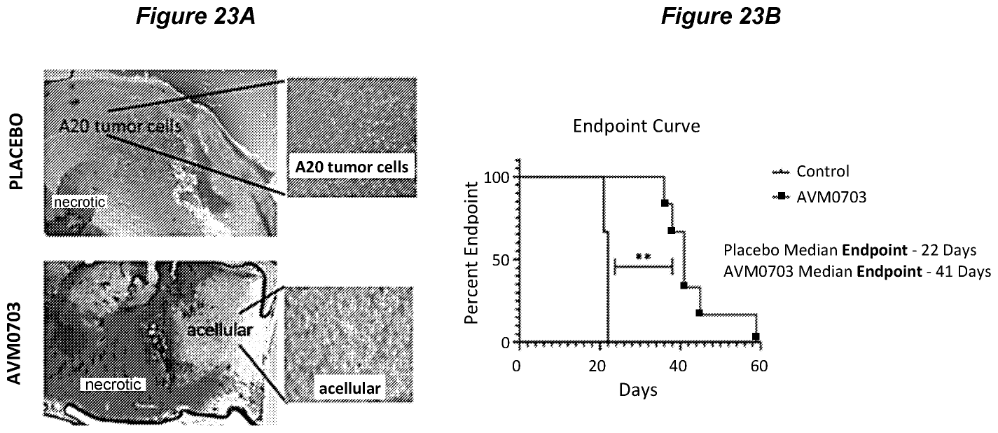

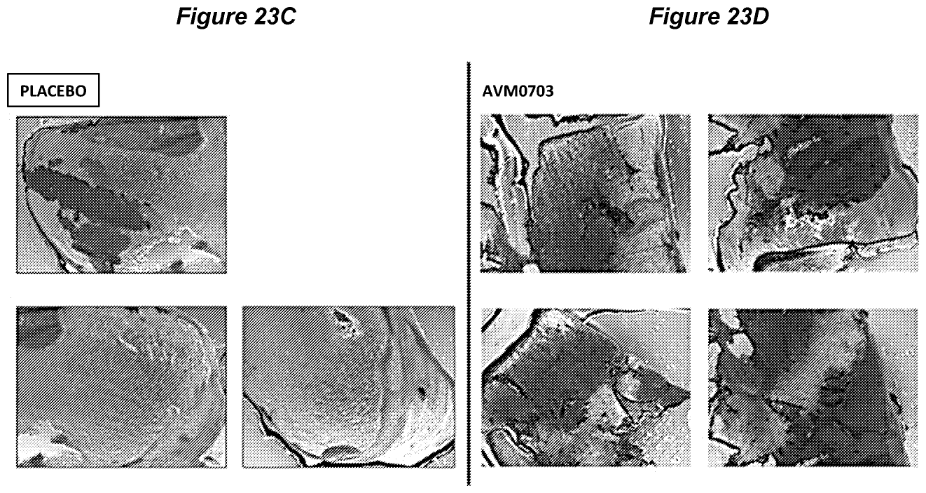

Tumor growth was delayed in mice treated with AVM0703 (FIG. 22). Mice treated with AVM0703 took approximately 2-fold longer to reach tumor volume endpoint than mice treated with placebo. AVM0703-treated mice had a median time to endpoint of 41 days while mice that only received placebo had a median time to endpoint of 22 days from the first day of dosing (FIG. 23).

Microscopic analysis revealed considerable differences in tumor structure in the AVM0703 treated mice compared to placebo. The placebo tumor had an open structure with an interior that displayed clear cellularity, indicating that the middle of the tumor was dense with tumor cells. The tumor treated with AVM0703 had a denser structure that showed extensive areas of necrosis. The middle of the treated tumor appeared to lack the presence of cells (FIG. 23).

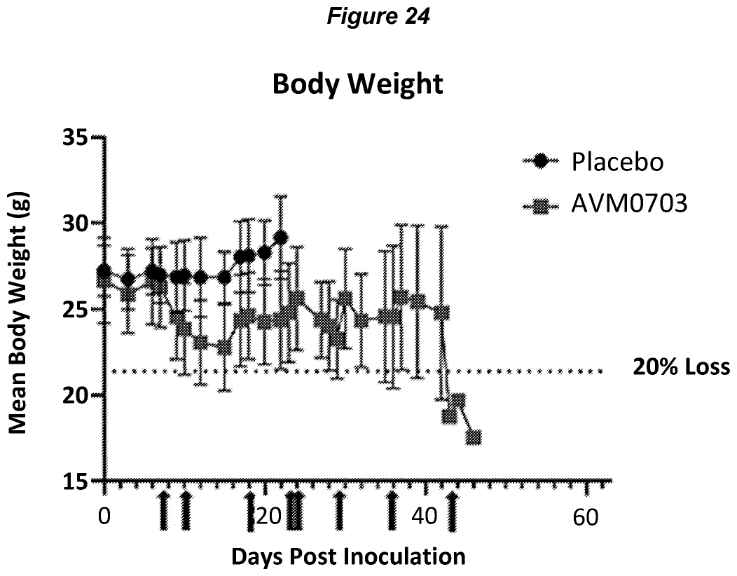

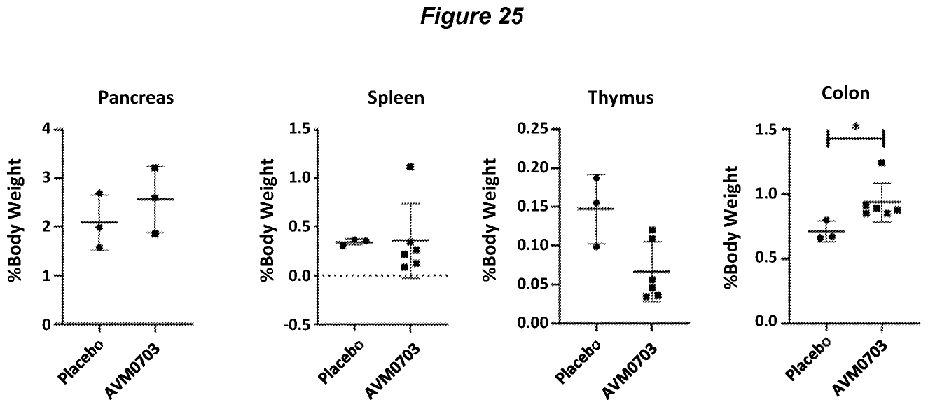

One mouse in the AVM treated group reached the 20% body weight loss threshold and was euthanized after 46 days and 8 doses of AVM0703 (FIG. 24). Group organ weights to body weight ratios were not significantly different at study endpoint for pancreas, thymus, and spleen; however, colon to body weight ratio was slightly increased in the AVM0703 treated group. Since the AVM0703 treated mice reached study endpoint between 14 and 40 days after the placebo treated mice reached study endpoint, the increase in colon weights may be due to the increased age of the mice (FIG. 25).

To determine whether changes in each mouse's spleen weight or thymus weight to body weight ratio might give an indication of continued responsiveness to AVM0703 with repeat dosing, the data were separated based on the number of days after AVM0703 dosing and the total number of AVM0703 doses received before reaching endpoint.

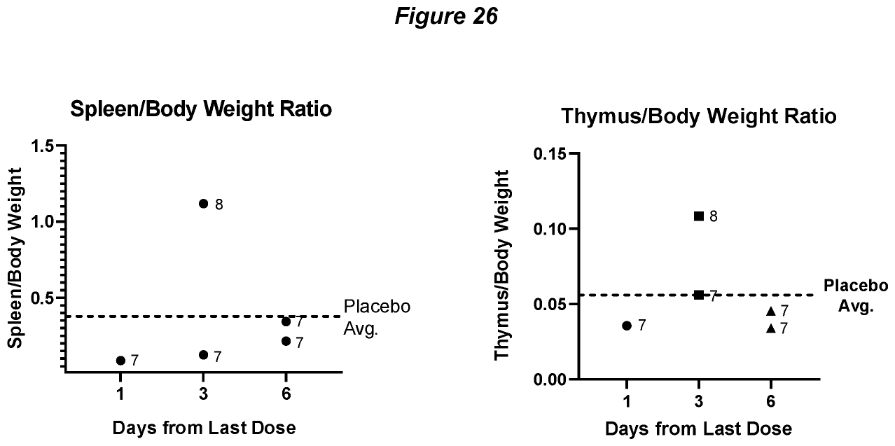

The results indicate that both the thymus and spleen remain responsive to repeat AVM0703 dosing for 7 doses, with responsiveness lost at the 8th dose (FIG. 26). On Days 1 and 3 after the 7th AVM0703 dose, both spleen and thymus weights appear reduced, with recovery, as expected, days after AVM0703 dosing.

### AVM_CANMOD_03

In a second study (“AVM_CANMOD_03”), BALB/c mice (n=5 per group) were dosed with increasing concentrations of AVM0703 HED DP (7, 18, and 26 mg/kg) on Days 9, 16, 24, and 31 after tumor inoculation. Treatment started when the tumors were approximately 500 mm³, as opposed to well-established tumors of 150 mm³ as in study AVM_CANMOD_01.

Similar efficacy was seen in the placebo and 7 mg/kg groups. The 18 and 26 mg/kg groups had similar efficacy and showed some separation from the low dose and placebo groups. No significant differences were found in the spleen, thymus, colon, or pancreas weights between any of the groups of mice at euthanization. The endpoint curve showed no significant differences in the median survival, though the mice that were treated with 18 and 26 mg/kg had approximately 6 more days of median survival than the placebo-treated mice. There were no sudden deaths of the mice.

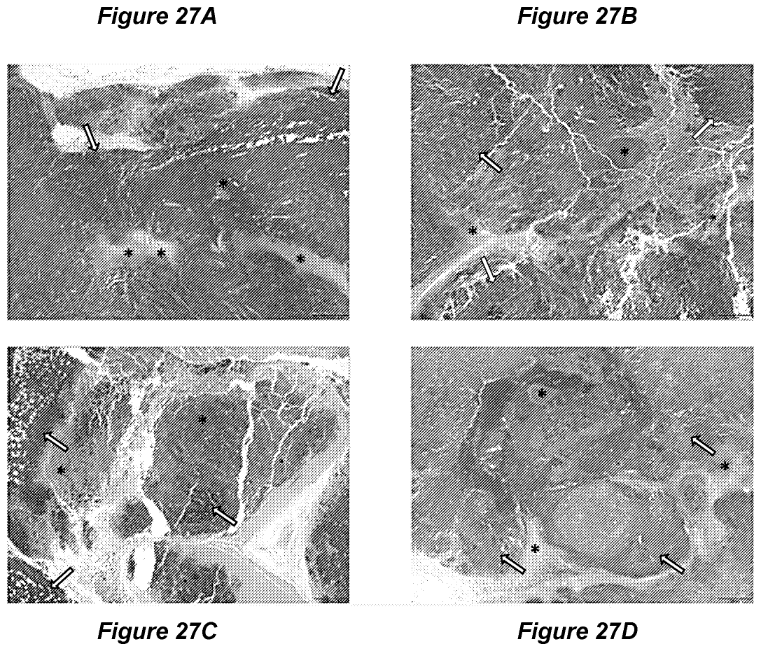

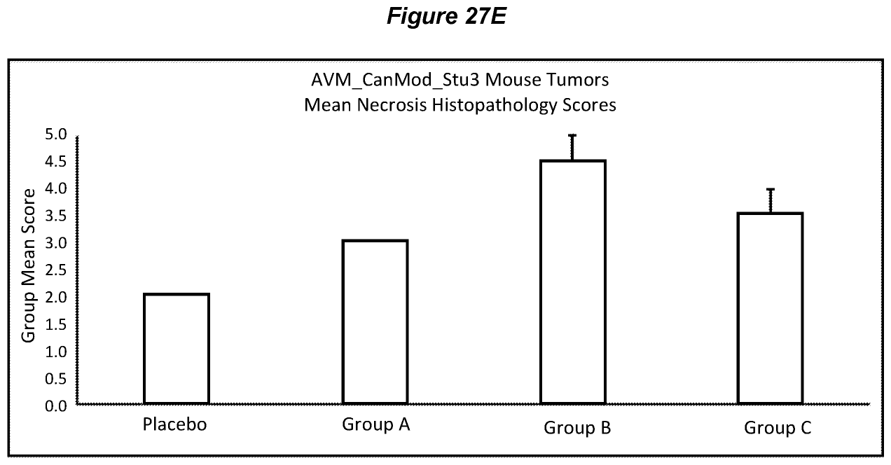

Based on evidence of pseudogrowth in tumors from AVM0703-treated mice in study AVM_CANMOD_01, tumors (n=2 per group) were sent to HistoTox Labs for slicing and immunohistochemistry. Hematoxylin and eosin stains identified several regions of necrosis and the slides were scored from 0 to 5 depending on severity. Tumors from mice treated with AVM0703 had larger areas of necrosis, and the mice dosed with 18 mg/kg DP had the greatest degree of necrosis as reflected in the scoring graph (FIG. 27C and FIG. 27E).

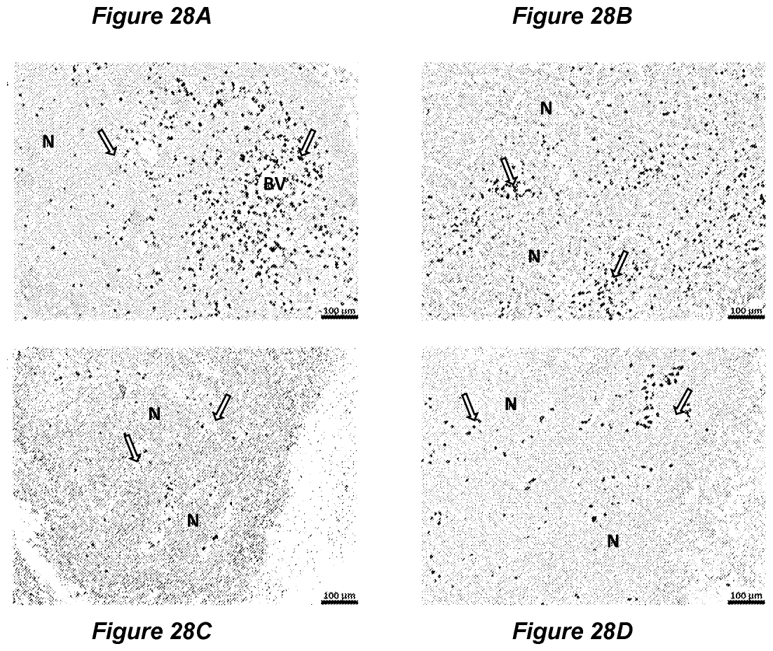

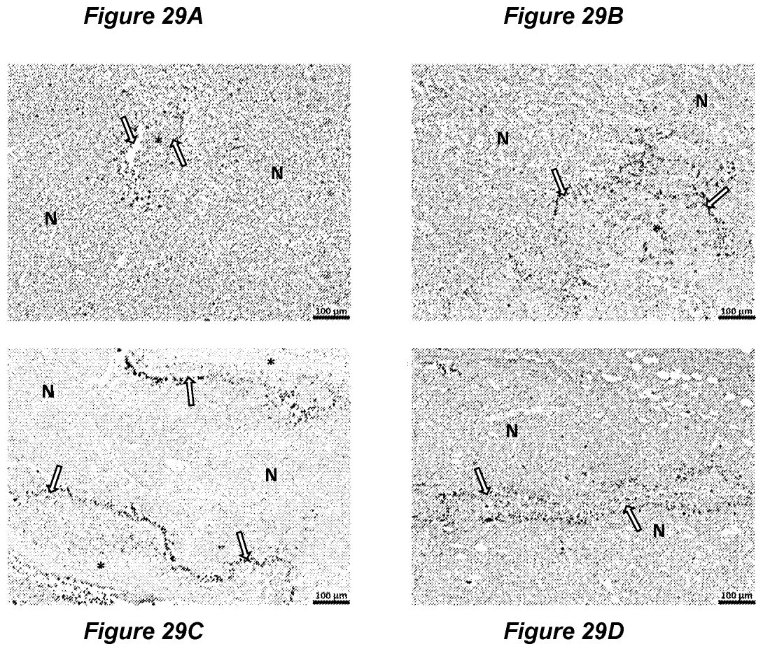

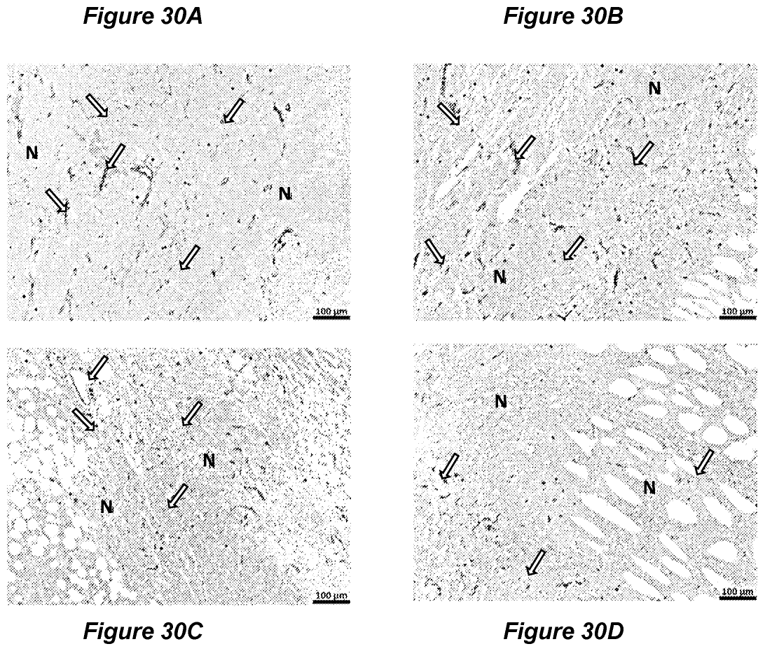

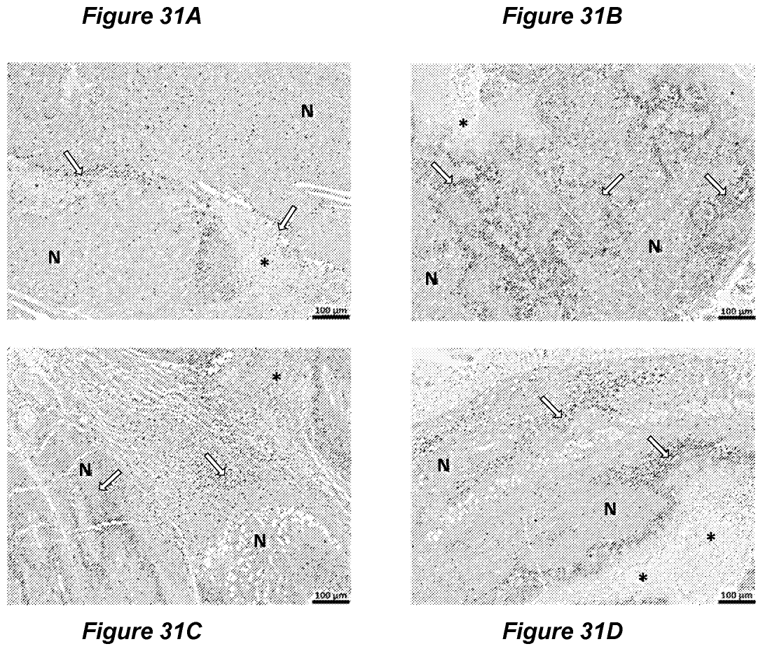

CD3 expression was greatest in the placebo mice and visually decreased with increasing concentrations of DP (FIG. 28). When staining for NKp46, an NK cell marker, there was a visual increase in cellular staining with increasing concentrations of DP. NK cells in the placebo-treated tumors (FIG. 29A) concentrated around blood vessels in the tumor. However, upon treatment with AVM0703, the NK cells localized to the edge between the neoplastic growth and the necrotic areas. From this, it was concluded that the NK cells are contributing to expanding necrosis within the tumor. Tumors stained for NK cell marker CD49b had high background and staining of epithelial tissue surrounding tumor micro vessels. There was a visual decrease in the staining of round cells, indicated by black arrows, with increased doses of AVM0703 (FIG. 30). Finally, tumor slices were stained for caspase 3, an apoptotic cellular marker. There was an increase in apoptotic staining with all doses of AVM0703 compared to placebo. This includes strong staining for apoptosis around necrotic regions, as well as isolated apoptosis in the areas of neoplastic growth (FIG. 31).

Taken together, treatment with AVM0703 resulted in an increase in NKp46 expressing cells that localized and most likely contributed to necrosis within the tumor. Activated NKT cells are known to lose both CD3 and CD49b expression, and thus, the elevated NK activity is most likely a combination of NK and NKT cell infiltration of the tumor. AVM0703 also induced increased apoptosis within the tumor (FIG. 31). This indicates that AVM0703 may trigger more than one tumor-killing mechanism.

### AVM_CANMOD_04

In a third study (“AVM_CANMOD_04”), BALB/c mice with established A20 tumors were dosed with both AVM0703 and chemotherapy (cyclophosphamide/fludarabine [Cy/Flu]). Mice were randomized into the following groups (n=3 to 5 per group): 1) placebo; 2) AVM0703 18 mg/kg HED DP per os (PO) (Day 11 and Day 14); 3) Cy/Flu (Day 11 and Day 14) [13.5 mg/kg/0.8 mg/kg, intraperitoneal (IP)/IP]; 4) AVM0703 18 mg/kg HED DP PO (Day 11) followed by Cy/Flu [13.5 mg/kg/0.8 mg/kg, IP/IP] (Day 14).

Tumor growth was reduced in mice who received 2 doses of Cy/Flu and in mice who received a combination of 1 dose of AVM0703 followed by 1 dose of Cy/Flu. The combination-treated mice had a median time to endpoint of 73 days after tumor inoculation, while none of the mice in the Cy/Flu group had reached endpoint at study close (95 days after tumor inoculation).

Tumors from this study were paraffin embedded and sectioned. Two section images from each tumor were forwarded to AVM. Images of the tumor sections were subsequently uploaded to MetaMorph Image Analysis Software. The percent of tumor that was dead was measured using Image Thresholding Software. The viable tumor area was subsequently calculated by subtracting the thresholded area from the total tumor area. All work was performed blinded to the group that the image belonged to.

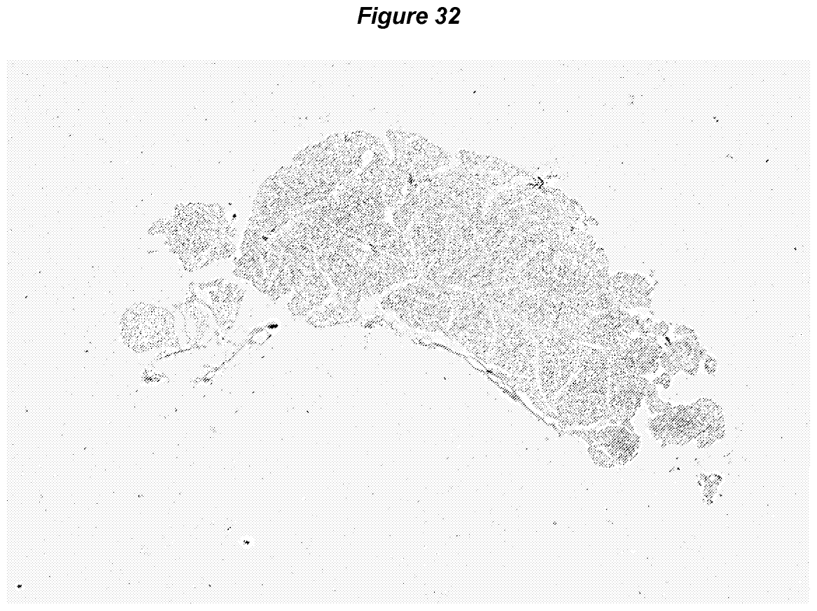

For the Cy/Flu group dosed on Days 11 and 14, 2 of the 3 mice did not have tumors at endpoint; however, the third mouse had clearly relapsed and had a very large tumor with a viable tumor area of 169,362 arbitrary units (AU). One mouse in the AVM0703 (Day 11) and Cy/Flu (Day 14) combination group had a tumor that was completely resorbed (FIG. 32) although, the area of the tumor was 182,279 AU, an example of pseudoprogression.

Two of the other mice in the combination group had tumors that were 90% necrotic and only had viable tumor areas of 10,000 to 25,000 AU. The average viable tumor area for the 5 mice in the combination group was only 16,490 AU, compared to an average viable tumor area for the placebo group of 104,318 AU, or compared to the 182,279 AU viable tumor area of the Cy/Flu mouse that relapsed. In this small study, the 18 mg/kg AVM0703 group had smaller viable tumor volumes (94,305 AU), but this was not significantly different from the placebo mice.

When compared to published results with CHOP (cyclophosphamide, hydroxydaunorubicin oncovin, prednisone) chemotherapy in the A20 model, AVM0703 combined with Cy/Flu induced remission longer than 1 cycle of CHOP chemotherapy, and maintained remission longer than 2 cycles of CHOP chemotherapy, where tumor escape was seen on about Day 42.

### AVM_CANMOD_05

A fourth study (“AVM_CANMOD_05”) was performed to examine how higher doses (18, 22, and 25 mg/kg HED DP) of AVM0703 affect the anti-tumor capabilities in the A20 B cell lymphoma mouse model.

Study AVM_CANMOD_05 was split into 2 subsets: lymphodepletion and endpoint analysis. Previous in-house lymphodepletion studies were conducted in naïve C57BL/6 mice, demonstrating the lymphodepleting effect of AVM0703 in healthy mice. To better understand the previous data suggesting AVM0703's anti-tumor effect and to better understand AVM0703's mechanism of action in the tumor model, it was necessary to illustrate the profile of lymphodepletion in the in vivo tumor models.

The lymphodepletion subset mice were euthanized 48 hours after dosing. The second subset, endpoint analysis, focused on examining the effect of repeated higher doses of AVM0703 on the time to endpoint of the study mice. Importantly, mice in this study were dosed when the A20 tumor was very large, about 390 mm³.

Checkpoint inhibitors, such as anti-PD-1 (eg, KEYTRUDA), are approved for clinical use in B cell lymphoma. In this very aggressive A20 B cell lymphoma model, the anti PD 1 is not effective if treatment is started after the tumors reach 100 mm³. Anti PD 1 is only effective in this model when treatment is started within 3 days of A20 inoculation, before tumors are even palpable.

HistoTox Labs scored the lymphodepletion subset. In the lymphodepletion subset, 2 of the 9 mice scored a 5 on necrosis (range 0 to 5). One of the 23 mice in the endpoint analysis subset had a tumor that was completely killed and resorbed, yielding an overall complete response rate of 9%—this is better than a 0% complete response rate to anti-PD-1 against established A20 tumors.

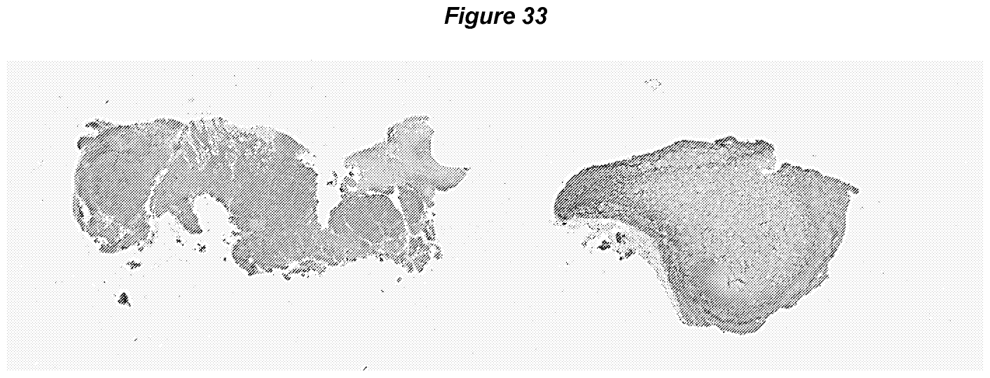

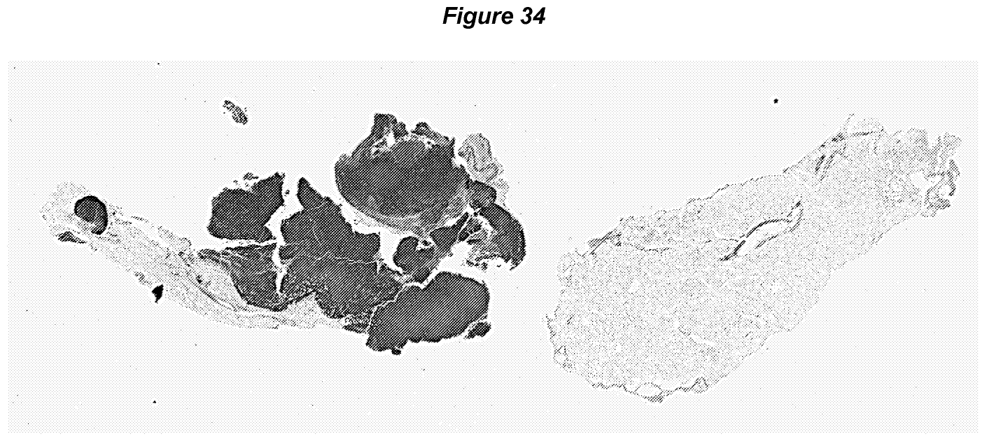

Tumor analysis using MetaMorph Image Analysis Software demonstrated that 18 and 25 mg/kg AVM0703 treated mice had greater tumor resorption than placebo treated mice (FIG. 33). However, in this study, the tumors had escaped and were growing, except for 1 mouse treated at 18 mg/kg whose tumor was 99.5% resorbed. Published research using the A20 model in an aggressive fashion like AVM has, demonstrates that even 2 cycles of CHOP, which directly kills 18% of the mice, also had 100% relapse within 20 days of complete remission.

HistoTox Labs scoring of the lymphodepletion subset demonstrated increased tumor necrosis (hematoxylin and eosin), reduced CD3 & CD49b label which can indicate activated immune cells, and increased Ly6G expression (a marker of AVM-NKT cells) in tumors from AVM0703 treated mice. For the endpoint analysis subset, tumors from the AVM0703 treated mice had reduced CD3 and CD49b label, increased organization of NKp46 cells (NK and NKT cells), and reduced Ly6G, Sca1, and collagen label. In the lymphodepletion subset, ALC was inversely related to AVM0703 dose. The lymphocytes that were not ablated at the 22 mg/kg and 25 mg/kg HED DP doses were primarily NK and NKT cells, and B cells. The 18 mg/kg HED DP dose almost completely ablated the B cell lymphocytes but did not ablate the NK and NKT cells. The different lymphodepletion profile may be due to mouse strain differences in sensitivity to AVM0703 or possibly due to differences between a naïve and tumor model.

Importantly, in the tumor model, glucose levels were not elevated in contrast to observations made in naïve C57BL/6 mice. At the 18 mg/kg HED, glucose levels were significantly reduced, although they did not reach hypoglycemic levels.

### Example 16—Acute High-Dose Dexamethasone has Inhibitory Effects in a CCRF-CEM Human T Cell Lymphoma Xenograft Model

A pilot study (“AVM_CANMOD_06”) was performed to investigate anti-tumor efficacy of AVM0703 in a human T cell lymphoma model, CCRF-CEM. Female NCr nude mice were inoculated with CCRF CEM human T-lymphoblasts and treated weekly with either oral placebo (n=2) or oral 18 mg/kg AVM0703 (n=3) after a 7 day implantation period.

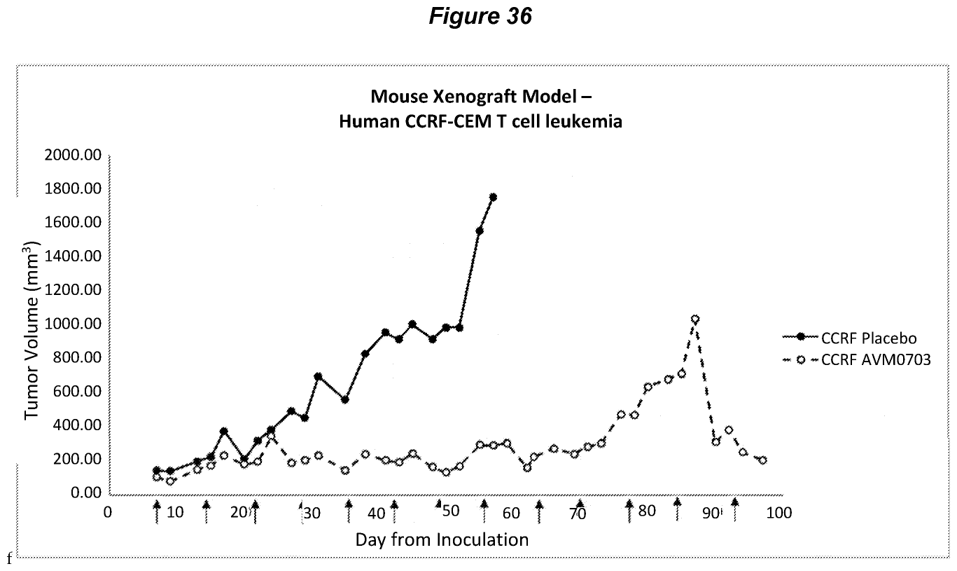

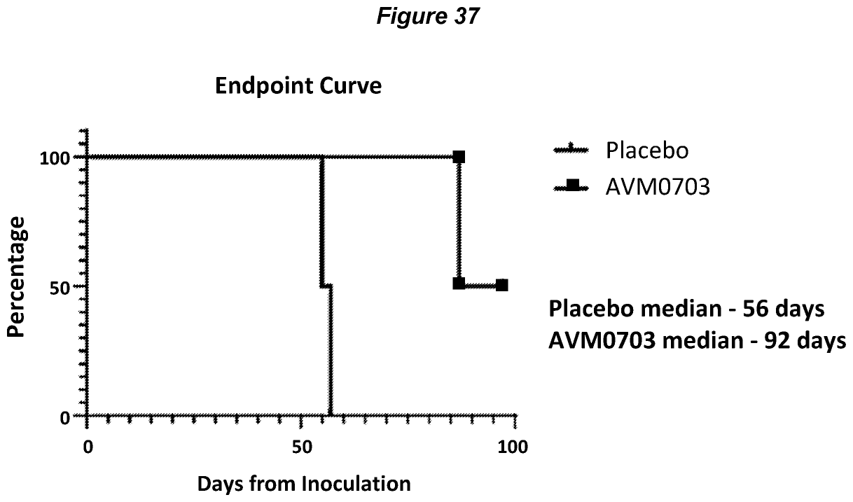

Tumor volume was assessed 3 times per week and endpoint was defined as either a tumor volume greater than 1500 mm³ or greater than a 20% loss from initial body weight measurement. Mice inoculated with CCRF-CEM cells showed a delay in time to endpoint when treated with AVM0703 compared to placebo (FIG. 36 and FIG. 37). Overall, there is a trend towards delayed tumor growth in CCRF CEM tumor bearing mice treated with AVM0703 compared to placebo.

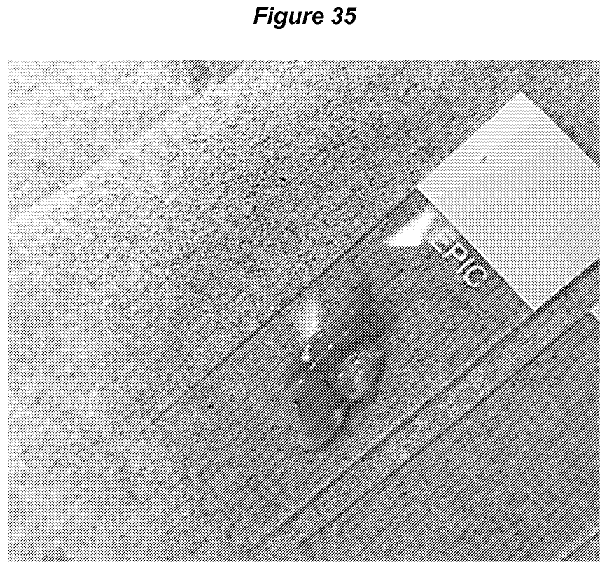

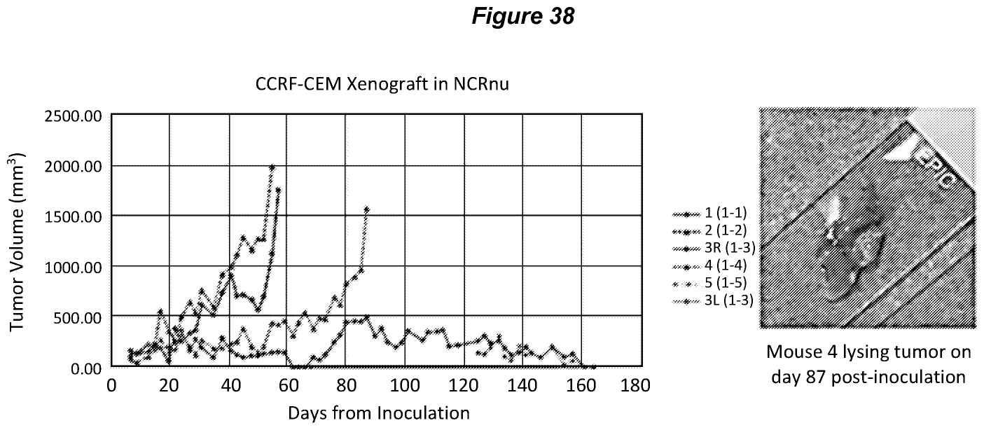

One mouse in the AVM0703 treated group was recently found dead on Day 89 after tumor inoculation—the tumor was removed and photographed (FIG. 35). Significant tumor lysis is apparent and most likely responsible for this mouse's death. An AVM0703 treated mouse 3R was re-challenged (3L) with human T-ALL (CCRF-CEM cell line) on day 118, and has no tumour growth out to day 164 (FIG. 38). Placebo mice reach tumour volume end point of 1500 mm³ on day 50-55. AVM0703 treated mice did not reach tumour volume end point.

### Example 17—Identification of AVM-NKT Cells in Human Subjects Treated with Acute High Doses of Dexamethasone

Following the identification of AVM-NKT cells in mice, data on file from human subjects treated with high doses of dexamethasone was re-analysed.

In osteoarthritis patients, under the guidelines of “Physician Practice of Medicine,” 3-6 mg/kg generic dexamethasone was administered (by Dr. Loniewski, Advanced Orthopedic Specialists, Brighton, Mich.) to 4 patients.

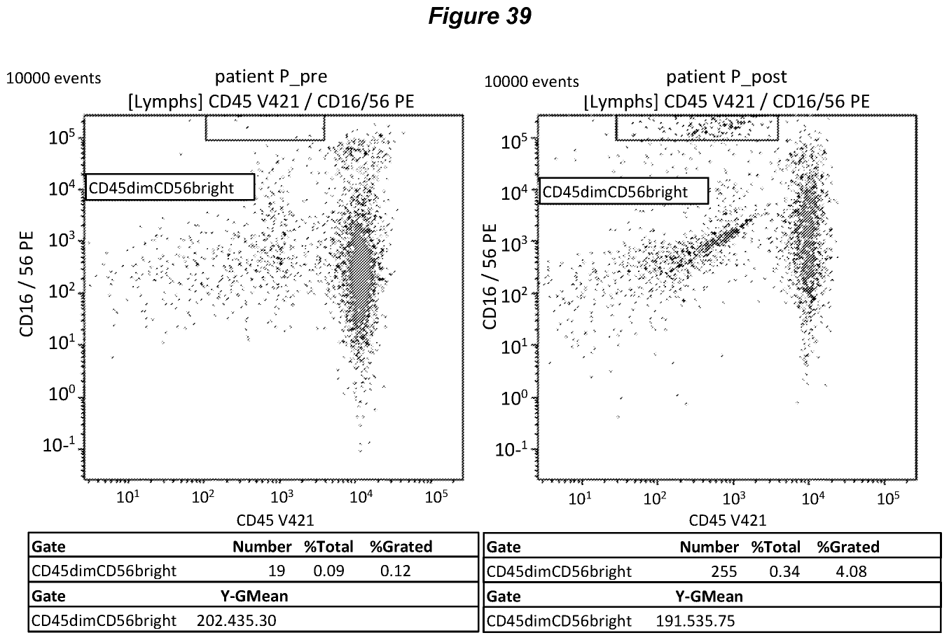

A review of flow cytometry data from the 4 patients taken 48 hours after they were treated with a dexamethasone dose that is 6-fold lower than the dose used to maximally induce AVM NKT in mice, was performed. CD45/CD56 scattergrams from one of the four patients shows that a new population of cells corresponding to the AVM-NKT cells identified in mice emerged—48 hours post treatment (FIG. 39).

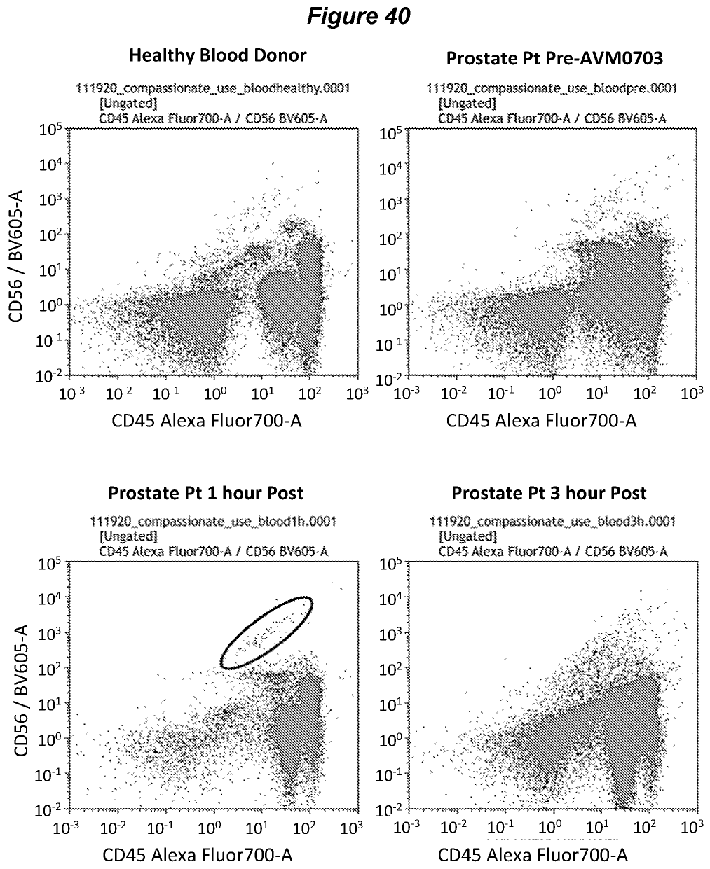

As shown in FIG. 40, a novel CD56 very bright cell population has also been observed in a prostate cancer patient one hour after his fourth AVM0703 treatment was infused at 6 mg/kg. The prostate cancer patient was a no-option patient after multi-year cancer treatment and has received a total of 4 AVM0703 infusions as least 28 days apart.

Compared to a healthy blood donor, the prostate cancer patient had evidence of a novel CD3 dim population, which was no longer evident one hour after AVM0703, however, a new CD56 very bright cell population was then evident in the blood which was no longer observed 3 hours after the infusion.

Compared to a healthy blood donor the prostate cancer patient had a CD3 dim and a NKp46dim population of cells pre-infusion, and one hour post-infusion of AVM0703 at 6 mg/kg the patient has a new CD56 very bright CD3dim population that was CD45 dim/negative and CD4/CD8 double negative.

### Example 18—Production and Mobilisation of Human AVM-NKT Cells in Humanized Mice

BRGSF humanized mice on a Balb/c background from Genoway generated by transplanting human umbilical cord blood CD34+ stem cells into irradiated mice that lack mouse B and T lymphocytes and NK cells but have a functional mouse complement system are orally dosed with HED 18-45 mg/kg DSP. 24-48 hours later human CD3high, and/or human CD45dim, and/or human CD56+ cells can be observed to be about 0.2-3% of total splenocytes by flow cytometry. The human CD3high, human CD45dim, and human CD56+ cells can be observed in the blood between about 36 hours out to 13 days later.

### HuCD34-NCG Mouse Model

The HuCD34-NCG mouse from Charles River is a study-ready mouse model with a human-like immune system, created by adoptive transfer of CD34+ stem cells. HuCD34-NCG mice are an ideal in vivo platform to evaluate the effectiveness of compounds modulating the human immune system. The lack, or late onset, of graft-versus-host disease (GvHD) in humanized mice make them ideal for long-term studies.

NCG mice are humanized by adoptive transfer using human umbilical cord blood-derived CD34+ stem cells, following myeloablation treatment. NCG mice from 4 donors (n=2 per donor) are orally dosed with HED 18-45 mg/kg DSP. 24-48 hours later human CD3high, and/or human CD45dim, and and/or human CD56+ cells can be observed to be about 0.2-3% of total splenocytes by flow cytometry. The human CD3high, human CD45dim, and human CD56+ cells can be observed in the blood between about 36 hours out to 13 days later.

huNOG EXL Mouse Model

huNOG EXL from Taconic have an average of 54% of CD45 cells positive for human CD45. Six huNOG EXL humanized immune system mice from 3 donors (n=2 per donor) are orally dosed with HED 18-45 mg/kg DSP. 24-48 hours later human CD3high, and/or human CD45dim, and and/or human CD56+ cells can be observed to be about 0.2-3% of total splenocytes by flow cytometry. The human CD3high, human CD45dim, and human CD56+ cells can be observed in the blood between about 36 hours out to 13 days later.

### Example 19—Venetoclax Pre-Treatment Dose-Dependently Reduces the Number of AVM-NKT Cells Mobilised into Blood Following High Dose Dexamethasone Administration

Female NOD mice of 10 weeks of age are treated with 12.5 mg/kg up to 50 mg/kg venetoclax 6-18 hours before oral gavage of a HED 30 mg/kg DSP dose. Venetoclax pretreatment dose-dependently reduces the number of CD3high, CD45dim, CD49b+ cells mobilized into the blood 48 hours after 30 mg/kg DSP dosing. CD3high, CD45dim, CD49b+ cells are reduced from—70 cells/microliter with DSP alone to—40 cells/microliter with 12.5 mg/kg venetoclax pretreatment, to—20 cells/microliter with 25 mg/kg venetoclax pretreatment, to—15 cells/microliter with 50 mg/kg venetoclax pretreatment. Venetoclax is a Bcl-2 inhibitor.

### Example 20—Acute High Dose Dexamethasone Prevents or Delays Hyperglycemia in Female Spontaneously Diabetic NOD Mice

Female NOD mice were ordered at 9 weeks of age. At 10 weeks of age when complete penetrance of insulitis in the pancreas is established, the mice were dosed with appropriate placebo for each treatment, or with cyclosporine twice weekly at 5 mg/kg for 7 weeks and then twice weekly at 10 mg/kg for the remainder of the 5 month study, or with a single acute oral single dose dexamethasone (AVM0703) at HED of 18 mg/kg or 30 mg/kg, or with venetoclax at 25 mg/kg, or with venetoclax at 25 mg/kg followed by dexamethasone at HED 30 mg/kg 18-24 hours later.

Body weights and blood glucose levels were monitored weekly. Body condition was monitored three times per week. Oral glucose tolerance was determined in all surviving mice who reached 30 weeks of age. Remaining mice were necropsied and autoreactive lymphocytes in the pancreas and adjacent lymph nodes were determined by flow cytometry. Pancreatitis was determined by H&E staining (8 out of 15 per group). Pancreatic Beta cell surface area was measured by staining for insulin. Insulin-secreting islets were scored as follows: 1, no insulitis (free of infiltration); 2, peri-insulitis (inflammatory cells outside or in the immediate vicinity of the islets); 3, insulitis (a clear and extensive islet infiltrate that shows direct lymphocyte-beta cell contact). Pancreas and pancreas lymph nodes were examined for autoreactive, insulin-specific CD4+ T cells by using a magnetic enrichment method together with tetramer reagents.

AVM0703 treated mice had significantly better body condition compared to all other groups of mice throughout the 5 month study. Venetoclax alone accelerated diabetes onset, which was delayed when AVM0703 was administered after the venetoclax dose. Alone, AVM0703 prevented diabetes in 40% of the mice and significantly delayed onset in the remaining 60% of the mice. Mice treated with AVM0703 without hyperglycemia at the end of the 5 month study had normal oral glucose tolerance tests (OGTT), while mice in all other groups had elevated glucose levels in response to fasting OGTT.

| | | | | Median Time to |
|---|---|---|---|---|
| | | First Onset of | % | diabetes (days |
| | | hyperglycemia | Hyperglycemic at | after 10 wks of |
| | N | (weeks of age) | 22 wks of age | age) |
| Placebo | 47 | 12 | 80% | 72 |
| AVM0703 | 15 | 20 | 60% | 113 |
| Cyclosporine | 14 | 18 | 70% | 86 |
| Venetoclax | 16 | 14 | 90% | 51 |
| Venetoclax + AVM0703 | 15 | 17 | 95% | 72 |

### Example 21—Acute High Dose Dexamethasone Reverses Diabetes in Early Onset and Established Diabetic Female NOD Mice

Female NOD mice are ordered at 9 weeks of age. Blood glucose levels are measured weekly starting at 10 weeks of age. Once a mouse has non-fasting blood glucose above 250 mg/dl, another measure is taken the following day.

For reversal of new-onset diabetes AVM0703 dosing is started one day after a mouse has had two consecutive days of elevated non-fasting blood glucose levels. Insulin pellets are implanted subcutaneously on the second day of elevated blood glucose levels. For reversal of established diabetes, two consecutive weeks of elevated blood glucose are measured, insulin pellets are implanted on day 8 after the first day of measured elevated blood glucose levels and AVM0703 is dosed on day 14 after the first day of measured elevated blood glucose levels.

Compared to anti-CD3 or ATG treated mice, AVM0703 is able to equivalently reverse both early onset and established diabetes without the body weight loss or poor body condition observed in anti-CD3 or ATG treated mice.

### REFERENCES

A number of publications are cited above in order to more fully describe and disclose the invention and the state of the art to which the invention pertains. Each reference is hereby incorporated by reference in its entirety. Full citations for these references are provided below:
Tarazona, Raquel & Peralbo, E. & Casado, Javier & Pena, Jose & Solana, Rafael. Human NKT cells in health and disease. Inmunologia. 2003; 22 Watarai H, Nakagawa R., Omori-Miyake M, Dashtsoodol N, & Taniguchi M. Methods for detection, isolation and culture of mouse and human invariant NKT cells. Nat Protoc. 2008; 3, 70-78.
Wolf B J, Choi J E, & Exley M A. Novel Approaches to Exploiting Invariant NKT Cells in Cancer Immunotherapy. Frontiers in immunology. 2018; 9, 384 Nair S, & Dhodapkar M V. Natural Killer T Cells in Cancer Immunotherapy. Frontiers in immunology. 2017; 8, 1178 Lodisch M. B. Clinical review: kinase inhibitors: adverse effects related to the endocrine system. The Journal of clinical endocrinology and metabolism. 2013; 98(4), 1333-1342
Bhullar, K S, Lagarón N O, McGowan E M. et al. Kinase-targeted cancer therapies: progress, challenges and future directions. Mol Cancer. 2018; 17, 48
Mato A R, Thompson M, Allan J N, Brander D M, Pagel J M, Ujjani C S, Nabhan C. Real-world outcomes and management strategies for venetoclax-treated chronic lymphocytic leukemia patients in the United States. Haematologica. 2018; 103(9), 1511-1517
Kadri S, Lee J, Fitzpatrick C, Galanina, N, Sukhanova, M, Venkataraman, G, Wang Y L. Clonal evolution underlying leukemia progression and Richter transformation in patients with ibrutinib-relapsed CLL. Blood advances. 2017; 1(12), 715-727
Mato A R, Nabhan C, Barr P M, Ujjani C S, Hill B T, Lamanna N, Skarbnik A P, Howlett C, Pu J J, Sehgal A R, Strelec L E, Vandegrift A, Fitzpatrick D M, Zent C S, Feldman T, Goy A, Claxton D F, Bachow S H, Kaur G O, Svoboda J, Nasta S D, Porter D, Landsburg D J, Schuster S J, Cheson B D, Kiselev P, Evens A M. Outcomes of CLL patients treated with sequential kinase inhibitor therapy: a real world experience. Blood. 2016; 128(18):2199-2205 Barrett A J, & Battiwalla M. Relapse after allogeneic stem cell transplantation. Expert review of hematology. 2010; 3(4), 429-441
Stadtfeld et al, Science. 2008 Nov. 7; 322(5903):945-9
Okita et al, Science. 2008 Nov. 7; 322(5903):949-53
Bowles et al, Dev Biol. 2000 Nov. 15; 227(2):239-55.
Chambers et al, Cell. 2003 May 30; 113(5):643-55.
Moss & Tang, 2003, Dev Biol. 2003 Jun. 15; 258(2):432-42.
Shiina et al, Immunology 2016; 150:127-138
Nakamura et al 2019 Int. J. Mol. Sci 20:4544
Kaczmarek et al 2017 Arch. Immunol. Ther. Exp. 65:201-214
WO 2018/183927
Patente et al; Front Immunol. 2018; 9: 3176.
Lee et al; PLoS One. 2018 Jan. 2; 13(1):e0190063.
Malloy et al; J Immunol. 2017 Jan. 1; 198(1):394-403.
Zheng et al; Cell Mol Immunol. 2013 January; 10(1):50-7.
Gogoi et al; Indian J Med Res. 2013 November; 138(5): 755-761.
Dong et al; Front Immunol. 2018 Dec. 4; 9:2812.
Zhao et al, 2018; J Immunol Res. 2018 Jul. 10; 2018:5
Pauza et al, 2018; Front Immunol. 2018; 9: 1305.
Himoudi et al, 2012; J Immunol. 2012 Feb. 15; 188(4):1708-16

### STATEMENTS OF DISCLOSURE

The following numbered statements, outlining aspects of the present disclosure, are part of the description.

### AVM-NKT Cells

101. A method of producing and/or mobilising a population of natural killer T cells (NKT cells), the method comprising administering to a subject a glucocorticoid-receptor (GR) modulating agent or ICAM3 modulating agent at a dose equivalent to about at least 6 mg/kg human equivalent dose (HED) of dexamethasone base,
   wherein the glucocorticoid receptor (GR) modulating agent or ICAM3 modulating agent induces and/or mobilises the population of NKT cells in the subject.

### NKT Cell Marker Expression

102. The method of statement 101, wherein the population of NKT cells are characterized in that at least 60, 70, 80, 90, 95, 96, 97, 98, or 99% of the cells:
   i) express CD3, CD4, CD8, CD45, CD49b, CD56, CD62L, NK1.1, Ly6G, Sca1, and/or TCR gamma/delta; and/or
   ii) do not express: C-kit, B220, FoxP3, and/or TCR alpha/beta.
103. The method of statement 102, wherein the NKT cells express:
   (i) CD3, CD4, CD45, CD49b, CD56, CD62L, NK1.1, Ly6G, and Sca1; or
   (ii) CD3, CD45, and CD56.
104. The method of any one of statements 102-103, wherein the NKT cells do not express C-kit, B220, FoxP3, or TCR alpha/beta.
105. The method according to any one of statements 102-104, wherein the NKT cells do not express CD8.
106. The method according to any one of statements 102-104, wherein the NKT cells:
   i) express CD4 and CD8; and/or
   ii) express Ly6G.
107. The method according to any one of statements 102-106, wherein the NKT cells are:
   i) CD4+/very bright;
   ii) CD8+/dim;
   iii) CD3+/very bright;
   iv) CD45+/dim;
   v) Sca1+/very bright;
   vi) CD44+/−;
   vii) CD69+/−;
   viii) CD25+/−; and/or
   ix) CD3+/very bright and CD45+dim and CD56+;
      optionally, wherein the expression levels are determined relative to the average expression level in a population of reference NKT cells, derived from a common source, which have not been contacted with the glucocorticoid-receptor (GR) modulating agent.
108. The method according to any one of statements 102-107, wherein expression is measured measured by flow cytometry, optionally wherein the flow cytometry is performed using the equipment, reagents, and/or conditions described herein (taken in isolation or in combination).

### Glucocorticoid

109. The method according to any one of statements 101-108, wherein the glucocorticoid-receptor (GR) modulating agent is a glucocorticoid, optionally wherein the glucocorticoid is selected from the group consisting of: dexamethasone, hydrocortisone, methylprednisolone, prednisone, prednisolone, prednylidene, cortisone, budesonide, betamethasone, flumethasone and beclomethasone.
110. The method according to statement 109, wherein the glucocorticoid is selected from the group consisting of: dexamethasone, betamethasone, and methylprednisone, preferably wherein the glucocorticoid is dexamethasone or betamethasone.
111. The method according to any one of statements 108-110, wherein the glucocorticoid is selected from the group consisting of dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone Isothiocyanatoethyl)Thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone.
112. The method according to statement 111, wherein the dexamethasone is dexamethasone sodium phosphate.

### Glucocorticoid Dose

113. The method according to any one of statements 101-112, wherein the glucocorticoid is administered at a dose equivalent to about:
   i) at least 6-12 mg/kg human equivalent dose (HED) of dexamethasone base;
   ii) at least 6 mg/kg human equivalent dose (HED) of dexamethasone base;
   iii) at least 12 mg/kg human equivalent dose (HED) of dexamethasone base;
   iv) at least 15 mg/kg human equivalent dose (HED) of dexamethasone base;
   v) at least 18 mg/kg human equivalent dose (HED) of dexamethasone base;
   vi) at least 24 mg/kg human equivalent dose (HED) of dexamethasone base;
   vii) 15 mg/kg human equivalent dose (HED) of dexamethasone base;
   viii) 24 mg/kg human equivalent dose (HED) of dexamethasone base;
   ix) 30 mg/kg human equivalent dose (HED) of dexamethasone base;
   x) 45 mg/kg human equivalent dose (HED) of dexamethasone base; or
   xi) a human equivalent dose (HED) of dexamethasone base taking a value in mg/kg from
      a range of mg/kg values, wherein said range is bound by two of the mg/kg values set forth in parts i) to x) above.
114. The method according to any one of statements 101-113, wherein the glucocorticoid is administered as a single acute dose, or as a total dose given over about a 72 hour period.
115. The method according to any one of statements 101-114, wherein the method comprises administering one or more further doses of a glucocorticoid.
116. The method according to statement 115, wherein the one or more further doses are administered:
   i) between 24 hours and 120 hours after a preceding glucocorticoid administration;
   ii) between 24 hours and 48 hours after a preceding glucocorticoid administration;
   iii) between 72 hours and 120 hours after a preceding glucocorticoid administration;
   iv) every 24, 48, 72, 96, 120, 144, or 168 hours after a first glucocorticoid administration;
   v) once every two weeks after a first glucocorticoid administration;
   vi) once monthly after a first glucocorticoid administration; or
   vii) twice weekly after a first glucocorticoid administration.

### NKT Cell Activation

117. The method according to any one of statements 101-116, further comprising a step of administering an NKT cell activator to the subject.
118. The method according to statement 117, wherein the NKT cell activator is selected from the group consisting of: alpha GalCer, Sulfatide, or an NKT-activating antibody.
119. The method according to statement 118, wherein the NKT cell activator isalpha GalCer loaded dendritic cells or monocytes.
120. The method according to any one of statements 117-119, wherein the NKT cell activator is administered within or around 48 hours after administration of glucocorticoid.

### Subject

121. The method according to any one of statements 101-120, wherein the subject is mammalian, preferably wherein the subject is human.
122. The method according to any one of statements 101-121, wherein the subject has, is suspected of having, or has been diagnosed with a disease selected from the group consisting of: cancer, autoimmune disease, or infectious disease.
123. The method according to statement 122, wherein the cancer is a solid tumour cancer.
124. The method according to statement 122, wherein the cancer is selected from the group consisting of: squamous cell cancer (such as epithelial squamous cell cancer); lung cancer, including small-cell lung cancer, non-small cell lung cancer, adenocarcinoma of the lung and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; and head and neck cancer.
125. The method according to statement 122, wherein the cancer is lymphoma, preferably a B cell lymphoma, a T cell lymphoma, or non Hodgkin lymphoma.
126. The method according to any one of statements 122-125, wherein the NKT cells treat the cancer via tumour infiltration.
127. The method according to statement 126, wherein the NKT cells treat the cancer via release of immune activating cytokines.
128. The method according to statement 126 or 127, wherein the NKT cells engulf and kill cancer cells.
129. The method according to any one of statements 126 to 128, wherein the NKT cells promote infiltration of other immune cells into the tumour.
130. The method according to any one of statements 126 to 129, wherein the NKT cells directly kill cancer cells via CD1d-directed apoptosis.
131. The method according to any one of statements 126 to 130, wherein the NKT cells cause tumour necrosis.
132. The method according to statement 122, wherein the autoimmune disease is selected from the group consisting of: multiple sclerosis, systemic sclerosis, amyotrophic lateral sclerosis, type 1 diabetes mellitus (T1D), scleroderma, pemphigus, and lupus.
133. The method according to statement 122, wherein the autoimmune disease is type 1 diabetes mellitus (T1D).
134. The method according to statement 122, wherein the infectious disease is selected from the group consisting of: HIV and herpes, hepatitis, human papilloma virus, or a disease resulting from infection with a coronavirus, such as COVID-19.
135. The method according to statement 122, wherein the infectious disease is:
   i) HIV; or
   ii) COVID-19.

### Isolation/Expansion Steps

136. The method according to any one of statements 101-135, further comprising a step of isolating a population of NKT cells from the subject or from a sample derived from the subject,
   optionally wherein the step of isolating is performed:
      i) at least 48 hours after glucocorticoid administration;
      ii) between 48 hours and 13 days after glucocorticoid administration; or
      iii) between 6 and 48 hours after glucocorticoid administration.
137. The method of statement 136, wherein the sample is selected from the group consisting of: blood, plasma, a tumor biopsy or surgically removed tumor, bone marrow, liver, and fat or adipose tissue.
138. The method according to statement 136 or 137, further comprising a step of expanding the isolated NKT cells.
139. The method according to any one of statements 136-138, further comprising a step of activating the isolated NKT cells with an NKT cell activator
   optionally wherein the NKT cell activator is selected from:
      i) a cytokine, a chemokine, a growth factor, and/or an NKT modulating agent;
      ii) alpha GalCer (alpha-Galactosylceramide; a-GalCer) sulfatide (3-O-sulfogalactosylceramide; SM4; sulfated galactocerebroside).

### Transfection of Isolated NKT Cells

140. The method according to any one of statements 136 to 139, further comprising a step of introducing a nucleic acid encoding a protein into the isolated NKT cells, and culturing the cells under conditions that facilitate expression of said protein.
141. The method according to statement 140, wherein the protein is selected from the group consisting of one or more of: a T-cell receptor (TCR), a chimeric antigen receptor (CAR), a split, universal and programmable CAR (SUPRA-CAR).
142. The method according to statement 141, wherein the CAR and/or TCR comprises an antigen-binding domain which binds to an antigen selected from the group consisting of: CD19, CD20, CD22, GD2, CD133, EGFR, GPC3, CEA, MUC1, Mesothelin, IL-13R, PSMA, ROR1, CAIX, Her2.
143. The method according to any one of statement 140-142, further comprising a step of expanding the NKT cells.
144. The method according to any one of statement 140-143, further comprising a step of activating the NKT cells with an NKT cell activator.

### Administration of Isolated NKT Cells

145. A method of treating cancer, autoimmune disease, or infectious disease in a subject, the method comprising administering a therapeutically effective dose of NKT cells isolated according to any one of statements 136 to 144, of the isolated NKT cells of any one of statements 401-406, or of the population of cells of statement 407, to the subject.
146. The method according to statement 145, wherein the subject to which the isolated NKT cells are administered is the same subject from which the NKT cells were isolated.
147. The method according to statement 145, wherein the subject to which the isolated NKT cells are administered is different to the subject from which the NKT cells were isolated.
148. The method according to any one of statements 145 to 147, wherein the NKT cells are administered to the subject by a method selected from the group consisting of: intravenous injection, intraperitoneal injection, intra-lymphatic injection, intrathecal injection, injection into the cerebrospinal fluid (CSF), direct injection into a tumour, and as a gel placed on or near a solid tumour.

### Medical Uses

149. A glucocorticoid for use in a method according to any one of statements 101-148.
150. Use of a glucocorticoid for the manufacture of a medicament for use in a method according to any one of statements 101-148.
151. Use of dexamethasone to induce and/or mobilise a population of NKT cells, wherein the population of NKT cells is induced and/or mobilised by a method according to any one of statements 101-148.
   AVM-NKT Derived iPSCs
152. A method of producing induced pluripotent stem cells (iPSCs), the method comprising reprogramming NKT cells isolated by a method according to any one of statements 136-138 to produce iPSCs.
153. The method of statement 152, wherein the reprogramming comprises introducing one or more expression cassettes encoding Oct3/4, Klf4, Sox2, and C-myc into the NKT cells.
154. The method of statement 152, wherein the reprogramming comprises introducing Oct3/4, KLF4, Sox2, and c-myc encoding mRNA into the NKT cells.
155. The method of statement 153 or 154, wherein the reprogramming further comprises introducing one or more expression cassettes encoding one or more of: Sox1, Sox3, Sox15, Klf1, Klf2, Klf5, L-myc, N-myc, Nanog, and/or LIN28 into the NKT cells.
156. The method of statement 153 or 154, wherein the reprogramming further comprises introducing one or more of: Sox1, Sox3, Sox15, Klf1, Klf2, Klf5, L-myc, N-myc, Nanog, and/or LIN28 encoding mRNA into the NKT cells.
157. The method according to any one of statement 152-156, further comprising inducing differentiation of the iPSCs.
158. The method according to statement 157, wherein the iPSCs are differentiated into NKT cells.
159. A method of producing a population of NKT cells, the method comprising differentiating iPSCs produced by a method according to any one of statement 152-156 into an NKT cell lineage.

### AVM-T Cells and AVM-Dendritic Cells

160. The method according to any one of statements 101-135, wherein the glucocorticoid receptor (GR) modulating agent also induces a population of T cells in the subject,
   optionally wherein the T cells are as defined in any one of statements 202-205.
161. The method according to any one of statements 101-135 or 160, wherein the glucocorticoid receptor (GR) modulating agent also activates a population of dendritic cells in the subject,
optionally wherein the dendritic cells are as defined in any one of statements 302-304.

### AVM-T Cells

201. A method of producing a population of T cells, the method comprising administering to a subject a glucocorticoid-receptor (GR) modulating agent or ICAM3 modulating agent at a dose equivalent to about at least 6 mg/kg human equivalent dose (HED) of dexamethasone base,
   wherein the glucocorticoid receptor (GR) modulating agent or ICAM3 modulating agent induces the population of T cells in the subject.

### T Cell Marker Expression

202. The method of statement 201, wherein the population of T cells are characterized in that at least 60, 70, 80, 90, 95, 96, 97, 98, or 99% of the cells:
   i) express CD3, CD4, CD45, and/or CD49b; and/or
   ii) do not express CDCD49b.
203. The method of statement 202, wherein the population of T cells are characterized in that at least 60, 70, 80, 90, 95, 96, 97, 98, or 99% of the cells express TCR gamma/delta.
204. The method according to any one of statements 202-203, wherein the T cells are CD3+/very bright,
   optionally, wherein the expression levels are determined relative to the average expression level in a population of reference T cells, derived from a common source, which have not been contacted with the glucocorticoid-receptor (GR) modulating agent.
205. The method according to any one of statements 202-204, wherein expression is measured by flow cytometry, optionally wherein the flow cytometry is performed using the equipment, reagents, and/or conditions described herein (taken in isolation or in combination).

### Glucocorticoid

206. The method according to any one of statements 201-205, wherein the glucocorticoid-receptor (GR) modulating agent is a glucocorticoid, optionally wherein the glucocorticoid is selected from the group consisting of: dexamethasone, hydrocortisone, methylprednisolone, prednisone, prednisolone, prednylidene, cortisone, budesonide, betamethasone, flumethasone and beclomethasone.
207. The method according to statement 206, wherein the glucocorticoid is selected from the group consisting of: dexamethasone, betamethasone, and methylprednisone, preferably wherein the glucocorticoid is dexamethasone or betamethasone.
208. The method according to any one of statements 206-207, wherein the glucocorticoid is selected from the group consisting of dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone Isothiocyanatoethyl)Thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone.
209. The method according to statement 208, wherein the dexamethasone is dexamethasone sodium phosphate.

### Glucocorticoid Dose

210. The method according to any one of statements 201-209, wherein the glucocorticoid is administered at a dose equivalent to about:
   i) at least 6-12 mg/kg human equivalent dose (HED) of dexamethasone base;
   ii) at least 6 mg/kg human equivalent dose (HED) of dexamethasone base;
   iii) at least 12 mg/kg human equivalent dose (HED) of dexamethasone base;
   iv) at least 15 mg/kg human equivalent dose (HED) of dexamethasone base;
   v) at least 18 mg/kg human equivalent dose (HED) of dexamethasone base;
   vi) at least 24 mg/kg human equivalent dose (HED) of dexamethasone base;
   vii) 15 mg/kg human equivalent dose (HED) of dexamethasone base;
   viii) 24 mg/kg human equivalent dose (HED) of dexamethasone base;
   ix) 30 mg/kg human equivalent dose (HED) of dexamethasone base;
   x) 45 mg/kg human equivalent dose (HED) of dexamethasone base; or
   xi) a human equivalent dose (HED) of dexamethasone base taking a value in mg/kg from
      a range of mg/kg values, wherein said range is bound by two of the mg/kg values set forth in parts i) to x) above.
211. The method according to any one of statements 201-210, wherein the glucocorticoid is administered as a single acute dose, or as a total dose given over about a 72 hour period.
212. The method according to any one of statements 201-211, wherein the method comprises administering one or more further doses of a glucocorticoid.
213. The method according to statement 212, wherein the one or more further doses are administered:
   viii) between 24 hours and 120 hours after a preceding glucocorticoid administration;
   ix) between 24 hours and 48 hours after a preceding glucocorticoid administration;
   x) between 72 hours and 120 hours after a preceding glucocorticoid administration;
   xi) every 24, 48, 72, 96, 120, 144, or 168 hours after a first glucocorticoid administration;
   xii) once every two weeks after a first glucocorticoid administration;
   xiii) once monthly after a first glucocorticoid administration; or
   xiv) twice weekly after a first glucocorticoid administration.

### T Cell Activation

214. The method according to any one of statements 201-213, further comprising a step of administering a T cell activator to the subject.
215. The method according to statement 214, wherein the T cell activator is a T cell activating antibody.
216. The method according to any one of statements 214-215, wherein the T cell activator is administered within or around 48 hours after administration of glucocorticoid.

### Subject

217. The method according to any one of statements 201-216, wherein the subject is mammalian, preferably wherein the subject is human.
218. The method according to any one of statements 201-217, wherein the subject has, is suspected of having, or has been diagnosed with a disease selected from the group consisting of: cancer, autoimmune disease, or infectious disease.
219. The method according to statement 218, wherein the cancer is a solid tumour cancer.
220. The method according to statement 218, wherein the cancer is selected from the group consisting of: squamous cell cancer (such as epithelial squamous cell cancer); lung cancer, including small-cell lung cancer, non-small cell lung cancer, adenocarcinoma of the lung and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; and head and neck cancer.
221. The method according to statement 218, wherein the cancer is lymphoma, preferably a B cell lymphoma, a T cell lymphoma, or non Hodgkin lymphoma.
222. The method according to any one of statements 218-221, wherein the T cells treat the cancer via tumour infiltration.
223. The method according to statement 222, wherein the T cells treat the cancer via release of immune activating cytokines.
224. The method according to any one of statements 222 to 223, wherein the T cells promote infiltration of other immune cells into the tumour.
225. The method according to any one of statements 222 to 224, wherein the T cells directly kill cancer cells by inducing apoptosis.
226. The method according to any one of statements 222 to 225, wherein the T cells cause tumour necrosis.
227. The method according to statement 218, wherein the autoimmune disease is selected from the group consisting of: multiple sclerosis, systemic sclerosis, amyotrophic lateral sclerosis, type 1 diabetes mellitus (T1D), scleroderma, pemphigus, and lupus.
228. The method according to statement 218, wherein the autoimmune disease is type 1 diabetes mellitus (T1D).
229. The method according to statement 218, wherein the infectious disease is selected from the group consisting of: HIV and herpes, hepatitis, human papilloma virus, or a disease resulting from infection with a coronavirus, such as COVID-19.
230. The method according to statement 218, wherein the infectious disease is:
   i) HIV; or
   ii) COVID-19.

### Isolation/Expansion Steps

231. The method according to any one of statements 201-230, further comprising a step of isolating a population of T cells from the subject or from a sample derived from the subject,
   optionally wherein the step of isolating is performed:
      i) at least 48 hours after glucocorticoid administration;
      ii) between 48 hours and 13 days after glucocorticoid administration; or
      iii) between 6 and 48 hours after glucocorticoid administration.
232. The method of statement 231, wherein the sample is selected from the group consisting of: blood, plasma, a tumor biopsy or surgically removed tumor, bone marrow, liver, and fat or adipose tissue.
233. The method according to statement 231 or 232, further comprising a step of expanding the isolated T cells.
234. The method according to any one of statements 231-233, further comprising a step of activating the isolated T cells with a T cell activator optionally wherein the T cell activator is a T cell activating antibody.

### Transfection of Isolated T Cells

235. The method according to any one of statements 231 to 234, further comprising a step of introducing a nucleic acid encoding a protein into the isolated T cells, and culturing the cells under conditions that facilitate expression of said protein.
236. The method according to statement 235, wherein the protein is selected from the group consisting of one or more of: a T-cell receptor (TCR), a chimeric antigen receptor (CAR), a split, universal and programmable CAR (SUPRA-CAR).
237. The method according to statement 236, wherein the CAR and/or TCR comprises an antigen-binding domain which binds to an antigen selected from the group consisting of: CD19, CD20, CD22, GD2, CD133, EGFR, GPC3, CEA, MUC1, Mesothelin, IL-13R, PSMA, ROR1, CAIX, Her2.
238. The method according to any one of statement 235-237, further comprising a step of expanding the T cells.
239. The method according to any one of statement 235-238, further comprising a step of activating the T cells with a T cell activator.

### Administration of Isolated T Cells

240. A method of treating cancer, autoimmune disease, or infectious disease in a subject, the method comprising administering a therapeutically effective dose of T cells isolated according to any one of statements 231 to 239, of the isolated T cells of any one of statements 408-413, or of the population of cells of statement 414, to the subject.
241. The method according to statement 240, wherein the subject to which the isolated T cells are administered is the same subject from which the T cells were isolated.
242. The method according to statement 240, wherein the subject to which the isolated T cells are administered is different to the subject from which the T cells were isolated.
243. The method according to any one of statements 240 to 242, wherein the T cells are administered to the subject by a method selected from the group consisting of: intravenous injection, intraperitoneal injection, intra-lymphatic injection, intrathecal injection, injection into the cerebrospinal fluid (CSF), direct injection into a tumour, and as a gel placed on or near a solid tumour.

### Medical Uses

244. A glucocorticoid for use in a method according to any one of statements 201-243.
245. Use of a glucocorticoid for the manufacture of a medicament for use in a method according to any one of statements 201-243.
246. Use of dexamethasone to induce a population of T cells, wherein the population of T cells is induced by a method according to any one of statements 201-243.
   AVM-T Cell Derived iPSCs
247. A method of producing induced pluripotent stem cells (iPSCs), the method comprising reprogramming T cells isolated by a method according to any one of statements 231-233 to produce iPSCs.
248. The method of statement 247, wherein the reprogramming comprises introducing one or more expression cassettes encoding Oct3/4, Klf4, Sox2, and C-myc into the T cells.
249. The method of statement 247, wherein the reprogramming comprises introducing Oct3/4, KLF4, Sox2, and c-myc encoding mRNA into the T cells.
250. The method of statement 248 or 249, wherein the reprogramming further comprises introducing one or more expression cassettes encoding one or more of: Sox1, Sox3, Sox15, Klf1, Klf2, Klf5, L-myc, N-myc, Nanog, and/or LIN28 into the T cells.
251. The method of statement 248 or 249, wherein the reprogramming further comprises introducing one or more of: Sox1, Sox3, Sox15, Klf1, Klf2, Klf5, L-myc, N-myc, Nanog, and/or LIN28 encoding mRNA into the T cells.
252. The method according to any one of statement 247-251, further comprising inducing differentiation of the iPSCs.
253. The method according to statement 252, wherein the iPSCs are differentiated into T cells.
254. A method of producing a population of T cells, the method comprising differentiating iPSCs produced by a method according to any one of statement 247-251 into an NKT cell lineage.

### AVM-T Cells and AVM-Dendritic Cells

255. The method according to any one of statements 201-230, wherein the glucocorticoid receptor (GR) modulating agent also induces a population of NKT cells in the subject,
   optionally wherein the NKT cells are as defined in any one of statements 102-108.
256. The method according to any one of statements 201-230 or 255, wherein the glucocorticoid receptor (GR) modulating agent also activates a population of dendritic cells in the subject,
   optionally wherein the dendritic cells are as defined in any one of statements 302-304.

AVM-Dendritic Cells
301. A method of producing a population of activated dendritic cells, the method comprising administering to a subject a glucocorticoid-receptor (GR) modulating agent or ICAM3 modulating agent at a dose equivalent to about at least 6 mg/kg human equivalent dose (HED) of dexamethasone base,
   wherein the glucocorticoid receptor (GR) modulating agent or ICAM3 modulating agent induces the population of dendritic cells in the subject.

### Dendritic Cell Marker Expression

302. The method of statement 301, wherein the population of dendritic cells are characterized in that at least 60, 70, 80, 90, 95, 96, 97, 98, or 99% of the cells express CD11b.
303. The method according to any one of statements 301-302, wherein the dendritic cells are CD11b+/very bright,
   optionally, wherein the expression levels are determined relative to the average expression level in a population of reference dendritic cells, derived from a common source, which have not been contacted with the glucocorticoid-receptor (GR) modulating agent.
304. The method according to any one of statements 302-303, wherein expression is measured by flow cytometry, optionally wherein the flow cytometry is performed using the equipment, reagents, and/or conditions described herein (taken in isolation or in combination).

### Glucocorticoid

305. The method according to any one of statements 301-304, wherein the glucocorticoid-receptor (GR) modulating agent is a glucocorticoid, optionally wherein the glucocorticoid is selected from the group consisting of: dexamethasone, hydrocortisone, methylprednisolone, prednisone, prednisolone, prednylidene, cortisone, budesonide, betamethasone, flumethasone and beclomethasone.
306. The method according to statement 305, wherein the glucocorticoid is selected from the group consisting of: dexamethasone, betamethasone, and methylprednisone, preferably wherein the glucocorticoid is dexamethasone or betamethasone.
307. The method according to any one of statements 305-306, wherein the glucocorticoid is selected from the group consisting of dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone Isothiocyanatoethyl)Thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone.
308. The method according to statement 307, wherein the dexamethasone is dexamethasone sodium phosphate.

### Glucocorticoid Dose

309. The method according to any one of statements 301-308, wherein the glucocorticoid is administered at a dose equivalent to about:
   i) at least 6-12 mg/kg human equivalent dose (HED) of dexamethasone base;
   ii) at least 6 mg/kg human equivalent dose (HED) of dexamethasone base;
   iii) at least 12 mg/kg human equivalent dose (HED) of dexamethasone base;
   iv) at least 15 mg/kg human equivalent dose (HED) of dexamethasone base;
   v) at least 18 mg/kg human equivalent dose (HED) of dexamethasone base;
   vi) at least 24 mg/kg human equivalent dose (HED) of dexamethasone base;
   vii) 15 mg/kg human equivalent dose (HED) of dexamethasone base;
   viii) 24 mg/kg human equivalent dose (HED) of dexamethasone base;
   ix) 30 mg/kg human equivalent dose (HED) of dexamethasone base;
   x) 45 mg/kg human equivalent dose (HED) of dexamethasone base; or
   xi) a human equivalent dose (HED) of dexamethasone base taking a value in mg/kg from
      a range of mg/kg values, wherein said range is bound by two of the mg/kg values set forth in parts i) to x) above.
310. The method according to any one of statements 301-309, wherein the glucocorticoid is administered as a single acute dose, or as a total dose given over about a 72 hour period.
311. The method according to any one of statements 301-310, wherein the method comprises administering one or more further doses of a glucocorticoid.
312. The method according to statement 311, wherein the one or more further doses are administered:
   i) between 24 hours and 120 hours after a preceding glucocorticoid administration;
   ii) between 24 hours and 48 hours after a preceding glucocorticoid administration;
   iii) between 72 hours and 120 hours after a preceding glucocorticoid administration;
   iv) every 24, 48, 72, 96, 120, 144, or 168 hours after a first glucocorticoid administration;
   v) once every two weeks after a first glucocorticoid administration;
   vi) once monthly after a first glucocorticoid administration; or
   vii) twice weekly after a first glucocorticoid administration.

### Dendritic Cell Activation

313. The method according to any one of statements 301-312, further comprising a step of administering a dendritic cell activator to the subject.
314. The method according to statement 313, wherein the dendritic cell activator is administered within or around 48 hours after administration of glucocorticoid.

### Subject

315. The method according to any one of statements 301-314, wherein the subject is mammalian, preferably wherein the subject is human.
316. The method according to any one of statements 301-315, wherein the subject has, is suspected of having, or has been diagnosed with a disease selected from the group consisting of: cancer, autoimmune disease, or infectious disease.
317. The method according to statement 316, wherein the cancer is a solid tumour cancer.
318. The method according to statement 316, wherein the cancer is selected from the group consisting of: squamous cell cancer (such as epithelial squamous cell cancer); lung cancer, including small-cell lung cancer, non-small cell lung cancer, adenocarcinoma of the lung and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; and head and neck cancer.
319. The method according to statement 318, wherein the cancer is lymphoma, preferably a B cell lymphoma, a T cell lymphoma, or non Hodgkin lymphoma.
320. The method according to any one of statements 317-319, wherein the dendritic cells treat the cancer via tumour infiltration.
321. The method according to statement 320, wherein the dendritic cells treat the cancer via release of immune activating cytokines.
322. The method according to any one of statements 320 to 321, wherein the dendritic cells promote infiltration of other immune cells, such as T cells, into the tumour.
323. The method according to any one of statements 320 to 322, wherein the dendritic cells promote tumour necrosis.
324. The method according to statement 316, wherein the autoimmune disease is selected from the group consisting of: multiple sclerosis, systemic sclerosis, amyotrophic lateral sclerosis, type 1 diabetes mellitus (T1D), scleroderma, pemphigus, and lupus.
325. The method according to statement 316, wherein the autoimmune disease is type 1 diabetes mellitus (T1D).
326. The method according to statement 316, wherein the infectious disease is selected from the group consisting of: HIV and herpes, hepatitis, human papilloma virus, or a disease resulting from infection with a coronavirus, such as COVID-19.
327. The method according to statement 316, wherein the infectious disease is:
   i) HIV; or
   ii) COVID-19.

### Isolation/Expansion Steps

328. The method according to any one of statements 301-327, further comprising a step of isolating a population of dendritic cells from the subject or from a sample derived from the subject, optionally wherein the step of isolating is performed:
   iv) at least 48 hours after glucocorticoid administration;
   v) between 48 hours and 13 days after glucocorticoid administration; or
   vi) between 6 and 48 hours after glucocorticoid administration.
329. The method of statement 328, wherein the sample is selected from the group consisting of: blood, plasma, a tumor biopsy or surgically removed tumor, bone marrow, liver, and fat or adipose tissue.
330. The method according to statement 328 or 329, further comprising a step of expanding the isolated dendritic cells.
331. The method according to any one of statements 328-330, further comprising a step of activating the isolated dendritic cells with a dendritic cell activator.

### Transfection of Isolated Dendritic Cells

332. The method according to any one of statements 328 to 331, further comprising a step of introducing a nucleic acid encoding a protein into the isolated dendritic cells, and culturing the cells under conditions that facilitate expression of said protein.
333. The method according to statement 332, wherein the protein is selected from the group consisting of one or more of: a T-cell receptor (TCR), a chimeric antigen receptor (CAR), a split, universal and programmable CAR (SUPRA-CAR).
334. The method according to statement 333, wherein the CAR and/or TCR comprises an antigen-binding domain which binds to an antigen selected from the group consisting of: CD19, CD20, CD22, GD2, CD133, EGFR, GPC3, CEA, MUC1, Mesothelin, IL-13R, PSMA, ROR1, CAIX, Her2.
335. The method according to any one of statement 332-334, further comprising a step of expanding the dendritic cells.
336. The method according to any one of statement 332-335, further comprising a step of activating the dendritic cells with a dendritic cell activator.

### Administration of Isolated Dendritic Cells

337. A method of treating cancer, autoimmune disease, or infectious disease in a subject, the method comprising administering a therapeutically effective dose of dendritic cells isolated according to any one of statements 328 to 336, of the isolated dendritic cells of any one of statements 415-420, or of the population of cells of statement 421, to the subject.
338. The method according to statement 337, wherein the subject to which the isolated dendritic cells are administered is the same subject from which the dendritic cells were isolated.
339. The method according to statement 337, wherein the subject to which the isolated dendritic cells are administered is different to the subject from which the dendritic cells were isolated.
340. The method according to any one of statements 337 to 339, wherein the dendritic cells are administered to the subject by a method selected from the group consisting of: intravenous injection, intraperitoneal injection, intra-lymphatic injection, intrathecal injection, injection into the cerebrospinal fluid (CSF), direct injection into a tumour, and as a gel placed on or near a solid tumour.

### Medical Uses

341. A glucocorticoid for use in a method according to any one of statements 301-340.
342. Use of a glucocorticoid for the manufacture of a medicament for use in a method according to any one of statements 301-340.
343. Use of dexamethasone to induce a population of dendritic cells, wherein the population of dendritic cells is induced by a method according to any one of statements 301-340.
   AVM-Dendritic Cell Derived iPSCs
344. A method of producing induced pluripotent stem cells (iPSCs), the method comprising reprogramming dendritic cells isolated by a method according to any one of statements 328-330 to produce iPSCs.
345. The method of statement 344, wherein the reprogramming comprises introducing one or more expression cassettes encoding Oct3/4, Klf4, Sox2, and C-myc into the dendritic cells.
346. The method of statement 344, wherein the reprogramming comprises introducing Oct3/4, KLF4, Sox2, and c-myc encoding mRNA into the dendritic cells.
347. The method of statement 345 or 346, wherein the reprogramming further comprises introducing one or more expression cassettes encoding one or more of: Sox1, Sox3, Sox15, Klf1, Klf2, Klf5, L-myc, N-myc, Nanog, and/or LIN28 into the dendritic cells.
348. The method of statement 345 or 346, wherein the reprogramming further comprises introducing one or more of: Sox1, Sox3, Sox15, Klf1, Klf2, Klf5, L-myc, N-myc, Nanog, and/or LIN28 encoding mRNA into the dendritic cells.
349. The method according to any one of statement 344-348, further comprising inducing differentiation of the iPSCs.
350. The method according to statement 349, wherein the iPSCs are differentiated into dendritic cells.
351. A method of producing a population of dendritic cells, the method comprising differentiating iPSCs produced by a method according to any one of statement 344-348 into a dendritic cell lineage.

### AVM-T Cells and AVM-Dendritic Cells

352. The method according to any one of statements 301-327, wherein the glucocorticoid receptor (GR) modulating agent also induces a population of NKT cells in the subject,
   optionally wherein the NKT cells are as defined in any one of statements 102-108.
353. The method according to any one of statements 301-327 or 352, wherein the glucocorticoid receptor (GR) modulating agent also activates a population of T cells in the subject,
   optionally wherein the T cells are as defined in any one of statements 202-205.
401. An isolated natural killer T cell (NKT cell) or population of natural killer T cells (NKT cell) produced by a method according to any one of statements 101-159.

402. An isolated natural killer T cell (NKT cell), characterized in that the cell expresses CD3, and:
i) expresses CD4, CD8, CD45, CD49b, CD56, CD62L, NK1.1, Ly6G, Sca1, and/or TCR gamma/delta; and/or
ii) does not express: C-kit, B220, FoxP3, and/or TCR alpha/beta.
403. The isolated NKT cell according to statement 402, wherein the NKT cell or its precursor has been isolated from a subject, wherein the NKT cell or a precursor of the NKT cell was contacted with a high dose glucocorticoid-receptor (GR) modulating agent either in vivo prior to isolation or in vitro after isolation, and wherein the level of CD3 expression is at least two times higher than the average level of CD3 expression in a population of reference NKT cells from the subject that have not been contacted with the GR modulating agent.
404. The isolated NKT cell according to statement 403, wherein the CD3 expression levels of said isolated NKT cell and said population of reference NKT cells are measured by flow cytometry.
405. The isolated NKT cell according to statement 404, wherein the flow cytometry is performed using the equipment, reagents, and/or conditions described herein (taken in isolation or in combination).
406. The isolated NKT cell according to any one of statements 403 to 405, wherein the level of CD3 expression of said isolated NKT cell is at least three times, at least four times, or at least five times higher than the average level of CD3 expression in said population of reference NKT cells from the subject that have not been contacted with the glucocorticoid-receptor (GR) modulating agent.
407. An isolated population of natural killer T cells (NKT cell), characterized in that at least 60, 70, 80, 90, 95, 96, 97, 98, or 99% of the cells:
   i) express CD3, CD4, CD8, CD45, CD49b, CD56, CD62L, NK1.1, Ly6G, Sca1, and/or TCR gamma/delta; and/or
   ii) do not express: C-kit, B220, FoxP3, and/or TCR alpha/beta.
408. An isolated T cell or population of T cells produced by a method according to any one of statements 201-254.
409. An isolated T cell, characterized in that the cell expresses CD3 and:
   i) expresses CD4, CD45, and/or CD49b; and/or
   ii) does not express CD8;
   optionally wherein the cell expresses TCR gamma/delta.
410. The isolated T cell according to statement 409, wherein the T cell or its precursor has been isolated from a subject, wherein the T cell or a precursor of the T cell was contacted with a high dose glucocorticoid-receptor (GR) modulating agent either in vivo prior to isolation or in vitro after isolation, and wherein the level of CD3 expression is at least two times higher than the average level of CD3 expression in a population of reference T cells from the subject that have not been contacted with the GR modulating agent.
411. The isolated T cell according to statement 410, wherein the CD3 expression levels of said isolated T cell and said population of reference T cells are measured by flow cytometry.
412. The isolated T cell according to statement 411, wherein the flow cytometry is performed using the equipment, reagents, and/or conditions described herein (taken in isolation or in combination).
413. The isolated T cell according to any one of statements 410 to 412, wherein the level of CD3 expression of said isolated T cell is at least three times, at least four times, or at least five times higher than the average level of CD3 expression in said population of reference T cells from the subject that have not been contacted with the glucocorticoid-receptor (GR) modulating agent.
414. An isolated population of T cells, characterized in that at least 60, 70, 80, 90, 95, 96, 97, 98, or 99% of the cells:
   i) express CD3, CD4, CD45, and/or CD49b; and/or
   ii) do not express CD8;
      and wherein the level of CD3 expression is at least three times, at least four times, or at least five times higher than the average level of CD3 expression in a population of reference T cells from the subject that have not been contacted with the glucocorticoid-receptor (GR) modulating agent;
   optionally wherein at least 60, 70, 80, 90, 95, 96, 97, 98, or 99% of the cells express TCR gamma/delta.
415. An isolated dendritic cell or population of dendritic cells produced by a method according to any one of statements 301-351.
416. An isolated dendritic cell, characterized in that the cell expresses CD11b.
417. The isolated dendritic cell according to statement 416, wherein the dendritic cell or its precursor has been isolated from a subject, wherein the dendritic cell or a precursor of the dendritic cell was contacted with a high dose glucocorticoid-receptor (GR) modulating agent either in vivo prior to isolation or in vitro after isolation, and wherein the level of CD11b expression is at least two times higher than the average level of CD11b expression in a population of reference dendritic cells from the subject that have not been contacted with the GR modulating agent.
418. The isolated dendritic cell according to statement 417, wherein the CD11b expression levels of said isolated dendritic cell and said population of reference dendritic cells are measured by flow cytometry.
419. The isolated dendritic cell according to statement 418, wherein the flow cytometry is performed using the equipment, reagents, and/or conditions described herein (taken in isolation or in combination).
420. The isolated dendritic cell according to any one of statements 417 to 419, wherein the level of CD11b expression of said isolated dendritic cell is at least three times, at least four times, or at least five times higher than the average level of CD11b expression in said population of reference dendritic cells from the subject that have not been contacted with the glucocorticoid-receptor (GR) modulating agent.
421. An isolated population of dendritic cells, characterized in that at least 60, 70, 80, 90, 95, 96, 97, 98, or 99% of the cells express CD11b; and wherein the level of CD11b expression is at least three times, at least four times, or at least five times higher than the average level of CD11b expression in a population of reference dendritic cells from the subject that have not been contacted with the glucocorticoid-receptor (GR) modulating agent.
422. A glucocorticoid for use in a method of treatment of cancer, autoimmune disease, or infectious disease in a subject, the method comprising administering a glucocorticoid to the subject at a dose equivalent to about 6-45 mg/kg human equivalent dose (HED) of dexamethasone base,
   wherein the glucocorticoid induces a population of NKT cells characterized in that at least 60, 70, 80, 90, 95, 96, 97, 98, or 99% of the cells:
   i) express CD3, CD4, CD8, CD45, CD49b, CD56, CD62L, NK1.1, Ly6G, Sca1, and/or TCR gamma/delta; and/or
   ii) do not express: C-kit, B220, FoxP3, and/or TCR alpha/beta.
423. A glucocorticoid for use in a method of treatment of cancer, autoimmune disease, or infectious disease in a subject, the method comprising administering a glucocorticoid to the subject at a dose equivalent to about 6-45 mg/kg human equivalent dose (HED) of dexamethasone base,
   wherein the glucocorticoid induces a population of T cells characterized in that at least 60, 70, 80, 90, 95, 96, 97, 98, or 99% of the cells express CD3, and wherein the level of CD3 expression is at least three times, at least four times, or at least five times higher than the average level of CD3 expression in a population of reference T cells from the subject that have not been contacted with the glucocorticoid-receptor (GR) modulating agent.
424. A glucocorticoid for use in a method of treatment of cancer, autoimmune disease, or infectious disease in a subject, the method comprising administering a glucocorticoid to the subject at a dose equivalent to about 6-45 mg/kg human equivalent dose (HED) of dexamethasone base,
   wherein the glucocorticoid activates a population of dendritic cells characterized in that at least 60, 70, 80, 90, 95, 96, 97, 98, or 99% of the cells express CD11b; and wherein the level of CD11b expression is at least three times, at least four times, or at least five times higher than the average level of CD11b expression in a population of reference dendritic cells from the subject that have not been contacted with the glucocorticoid-receptor (GR) modulating agent.
425. A glucocorticoid for use in a method of treatment of cancer, autoimmune disease, or infectious disease in a subject, the method comprising administering a glucocorticoid to the subject at a dose equivalent to about 6-45 mg/kg human equivalent dose (HED) of dexamethasone base,
   wherein the glucocorticoid:
   i) induces a population of natural killer T cells (NKT cell) as defined in any one of statements 101-159;
   ii) induces a population of T cells as defined in any one of statements 201-254; and/or
   iii) activates a population of dendritic cells as defined in any one of statements 301-351.
426. A method of treating cancer, autoimmune disease, or infectious disease in a subject, the method comprising administering a therapeutically effective dose of:
   i) NKT cells isolated according to any one of statements 136 to 144, of isolated NKT cells of any one of statements 401-406, or of the population of cells of statement 407;
   ii) T cells isolated according to any one of statements 231 to 239, of the isolated T cells of any one of statements 408-413, or of the population of cells of statement 414, to the subject; and/or
   iii) dendritic cells isolated according to any one of statements 328 to 336, of the isolated dendritic cells of any one of statements 415-420, or of the population of cells of statement 421;
   to the subject.
501. A method of treating a disease resulting from infection with a coronavirus in a subject, the method comprising administering a glucocorticoid-receptor (GR) modulating agent to the subject at a dose equivalent to about at least 6 mg/kg human equivalent dose (HED) of dexamethasone base.
502. The method according to statement 501, wherein the glucocorticoid-receptor (GR) modulating agent is a glucocorticoid.
503. The method according to statement 501, wherein the glucocorticoid-receptor (GR) modulating agent is dexamethasone or betamethasone.
504. The method according to any one of statements 501-503, wherein the glucocorticoid-receptor modulating agent is administered at a dose equivalent to about at least 18 mg/kg human equivalent dose (HED) of dexamethasone base.
505. The method according to any one of statements 501-504, wherein the glucocorticoid-receptor modulating agent is administered at a dose equivalent to between about 18 mg/kg and 30 mg/kg human equivalent dose (HED) of dexamethasone base.
506. The method according to any one of statements 501-505, wherein the disease is COVID-19.
507. The method according to any one of statements 501-506, wherein the glucocorticoid-receptor modulating agent:
   i) induces a population of natural killer T cells (NKT cell) as defined in any one of statements 101-159;
   ii) induces a population of T cells as defined in any one of statements 201-254; and/or
   iii) activates a population of dendritic cells as defined in any one of statements 301-351.
508. The method according to statement 507, wherein the NKT cells treat the disease via engulfing and killing the coronavirus, and/or by activating other innate and adaptive immune cells.
509. A glucocorticoid-receptor (GR) modulating agent for use in a method according to any one of statements 501-508.
510. Use of a glucocorticoid-receptor (GR) modulating agent for the manufacture of a medicament for use in a method according to any one of statements 501-508.

1. A method of producing a population of natural killer T cells (NKT cells), the method comprising administering to a subject a glucocorticoid-receptor (GR) modulating agent at a dose equivalent to about at least 6 mg/kg human equivalent dose (HED) of dexamethasone base.

2. The method according to claim 1, wherein the population of NKT cells are characterized in that at least 60, 70, 80, 90, 95, 96, 97, 98, or 99% of the cells:
i) express CD3; and
ii) express CD4, CD8, CD45, CD49b, CD56, CD62L, NK1.1, Ly6G, Sca1, and/or TCR gamma/delta; and/or
iii) do not express: C-kit, B220, FoxP3, and/or TCR alpha/beta.

3. The method according to claim 2, wherein the NKT cells express:
i) CD3, CD4, CD45, CD49b, CD56, CD62L, NK1.1, Ly6G, Sca1, and TCR gamma/delta; or
ii) CD3, CD45, and CD56.

4. The method according to claim 2 or 3, wherein the NKT cells:
i) do not express C-kit, B220, FoxP3, or TCR alpha/beta,
ii) do not express CD8,
iii) express CD4 and CD8;
iv) express Ly6G and TCR gamma/delta; and/or
v) are CD3+ very bright and/or CD45+/dim and/or CD56+.

5. The method according to any one of claims 1-4, wherein the glucocorticoid-receptor (GR) modulating agent is a glucocorticoid, optionally wherein the glucocorticoid is selected from the group consisting of: dexamethasone, hydrocortisone, methylprednisolone, prednisone, prednisolone, prednylidene, cortisone, budesonide, betamethasone, flumethasone and beclomethasone.

6. The method according to claim 5, wherein the glucocorticoid is selected from the group consisting of: dexamethasone, betamethasone, and methylprednisone.

7. The method according to claim 6, wherein the glucocorticoid is dexamethasone or betamethasone.

8. The method according to any one of claims 5-7, wherein the dexamethasone is dexamethasone sodium phosphate.

9. The method according to any one of claims 1-8, wherein the glucocorticoid is administered at a dose equivalent to about:
i) at least 6-12 mg/kg human equivalent dose (HED) of dexamethasone base;
ii) at least 6 mg/kg human equivalent dose (HED) of dexamethasone base;
iii) at least 12 mg/kg human equivalent dose (HED) of dexamethasone base;
iv) at least 15 mg/kg human equivalent dose (HED) of dexamethasone base;
v) at least 21 mg/kg human equivalent dose (HED) of dexamethasone base;
vi) at least 24 mg/kg human equivalent dose (HED) of dexamethasone base;
vii) 15 mg/kg human equivalent dose (HED) of dexamethasone base;
viii) 24 mg/kg human equivalent dose (HED) of dexamethasone base; or
ix) 45 mg/kg human equivalent dose (HED) of dexamethasone base.

10. The method according to any one of claims 1-9, wherein the glucocorticoid is administered as a single acute dose, or as a total dose given over about a 72 hour period.

11. The method according to any one of claims 1-10, wherein the method comprises administering one or more further doses of a glucocorticoid.

12. The method according to any one of claims 1-11, further comprising a step of administering an NKT cell activator to the subject.

13. The method according to claim 12, wherein the NKT cell activator is administered within or around 48 hours after administration of glucocorticoid.

14. The method according to any one of claims 1-13, wherein the subject has, is suspected of having, or has been diagnosed with a disease selected from the group consisting of: cancer, autoimmune disease, or infectious disease.

15. The method according to claim 14, wherein the cancer is a solid tumour cancer.

16. The method according to claim 15, wherein the cancer is lymphoma, preferably a B cell lymphoma, a T cell lymphoma, or non Hodgkin lymphoma.

17. The method according to any one of claims 14-16, wherein the NKT cells treat the cancer via tumour infiltration.

18. The method according to any one of claims 14-17, wherein the NKT cells promote infiltration of other immune cells into the tumour.

19. The method according to any one of claims 14-18, wherein the NKT cells directly kill cancer cells via CD1d-directed apoptosis.

20. The method according to any one of claims 14-19, wherein the NKT cells treat the cancer by causing tumor necrosis.

21. The method according to claim 14, wherein the autoimmune disease is selected from the group consisting of: multiple sclerosis, systemic sclerosis, amyotrophic lateral sclerosis, type 1 diabetes mellitus (T1D), scleroderma, pemphigus, and lupus.

22. The method according to claim 14, wherein the infectious disease is HIV or a disease resulting from infection with a coronavirus, such as COVID-19.

23. The method according to any one of claims 1-22, further comprising a step of isolating a population of NKT cells from the subject or from a sample derived from the subject,
optionally wherein the step of isolating is performed:
i) at least 48 hours after glucocorticoid administration; or
ii) between 48 hours and 13 days after glucocorticoid administration.

24. The method of claim 23, wherein the sample is selected from the group consisting of: blood, plasma, a tumor biopsy or surgically removed tumor, bone marrow, liver, and fat or adipose tissue.

25. The method according to claim 23 or 24, further comprising a step of expanding the isolated NKT cells.

26. The method according to any one of claims 23-25, further comprising a step of activating the isolated NKT cells with an NKT cell activator
optionally wherein the NKT cell activator is selected from a cytokine, a chemokine, a growth factor, and/or an NKT modulating agent.

27. The method according to any one of claims 23-25, further comprising a step of introducing a nucleic acid encoding a protein into the isolated NKT cells, and culturing the cells under conditions that facilitate expression of said protein.

28. The method according to claim 27, wherein the protein is selected from the group consisting of one or more of: a T-cell receptor (TCR), a chimeric antigen receptor (CAR), and a split, universal and programmable CAR (SUPRA-CAR).

29. The method according to any one of claims 23-28, further comprising a step of expanding the NKT cells.

30. The method according to any one of claims 23-29, further comprising a step of activating the NKT cells with an NKT cell activator.

31. A method of treating cancer, autoimmune disease, or infectious disease in a subject, the method comprising administering a therapeutically effective dose of the isolated NKT cells of any one of claims 1-30 to the subject.

32. A glucocorticoid for use in a method according to any one of claims 1-31.

33. Use of a glucocorticoid for the manufacture of a medicament for use in a method according to any one of claims 1-31.

34. An isolated natural killer T cell (NKT cell) or population of natural killer T cells (NKT cell) produced by a method according to any one of claims 1-33.

35. An isolated natural killer T cell (NKT cell), characterized in that the cell expresses CD3, and:
i) expresses CD4, CD8, CD45, CD49b, CD56, CD62L, NK1.1, Ly6G, Sca1, and/or TCR gamma/delta; and/or
ii) does not express: C-kit, B220, FoxP3, and/or TCR alpha/beta;
optionally wherein the isolated NKT cell is CD3+ very bright and/or CD45+/dim and/or CD56+.

36. The isolated NKT cell according to claim 35, wherein the NKT cell or its precursor has been isolated from a subject, wherein the NKT cell or a precursor of the NKT cell was contacted with a high dose glucocorticoid receptor modulating agent either in vivo prior to isolation or in vitro after isolation, and wherein the level of CD3 expression is at least two times higher than the average level of CD3 expression in a population of reference NKT cells from the subject that have not been contacted with the glucocorticoid receptor modulating agent.

37. The isolated NKT cell according to claim 36, wherein the CD3 expression levels of said isolated NKT cell and said population of reference NKT cells are measured by flow cytometry.

38. The isolated NKT cell according to claim 36 or 37, wherein the level of CD3 expression of said isolated NKT cell is at least three times, at least four times, or at least five times higher than the average level of CD3 expression in said population of reference NKT cells from the subject that have not been contacted with the glucocorticoid receptor modulating agent.

39. An isolated population of natural killer T cells (NKT cell), characterized in that at least 60, 70, 80, 90, 95, 96, 97, 98, or 99% of the cells:
i) express CD3, CD4, CD8, CD45, CD49b, CD56, CD62L, NK1.1, Ly6G, Sca1, and/or TCR gamma/delta; and/or
ii) do not express: C-kit, B220, FoxP3, and/or TCR alpha/beta;
optionally wherein at least 60, 70, 80, 90, 95, 96, 97, 98, or 99% of the cells are CD3+ very bright and/or CD45+/dim and/or CD56+.

40. A glucocorticoid for use in a method of treatment of cancer, autoimmune disease, or infectious disease in a subject, the method comprising administering a glucocorticoid to the subject at a dose equivalent to about 6-45 mg/kg human equivalent dose (HED) of dexamethasone base,
wherein the glucocorticoid induces a population of NKT cells characterized in that at least 60, 70, 80, 90, 95, 96, 97, 98, or 99% of the cells:
i) express CD3, CD4, CD8, CD45, CD49b, CD56, CD62L, NK1.1, Ly6G, Sca1, and/or TCR gamma/delta; and/or
ii) do not express: C-kit, B220, FoxP3, and/or TCR alpha/beta.
